(19) United States
(12) Patent Application Publication
(10) Pub. No.: US 2006/0084825 A1
(43) Pub. Date: Apr. 20, 2006

(54) Novel method for the stereoselective synthesis of cyclic amino acids

(75) Inventors: Justin Stephen Bryans, Balsham (GB); David Clive Blakemore, Cambridge (GB); Sophie Caroline Williams, Cambridge (GB)

Correspondence Address: SCULLY SCOTT MURPHY & PRESSER, PC, 400 GARDEN CITY PLAZA, SUITE 300, GARDEN CITY, NY, 11530, US

(73) Assignee: Warner-Lambert Company, Morris Plains, NJ (US)

(21) Appl. No.: 10/968,430

(22) Filed: Oct. 19, 2004

(51) Int. Cl. C07C 227/10

(52) U.S. Cl. 562/504

(57) ABSTRACT

The instant invention is a route to stereospecific 3-substituted 5-membered ring isomers of Formula (A). The final products are useful as agents in the treatment of epilepsy, faintness attacks, hypokinesia, cranial disorders, neurodegenerative disorders, depression, anxiety, panic, pain, neuropathological disorders, gastrointestinal disorders such as irritable bowel syndrome (IBS), inflammation especially arthritis, sleep disorders, premenstrual syndrome, and hot flashes. The invention provides novel routes to synthesize stereoselectively analogs of gabapentin (Neurontin™) of Formulas (I), (II), (III), and (IV) wherein R is C₁-C₁₀ alkyl or C₃ -C₁₀ cycloalkyl and pharmaceutically acceptable salts thereof.

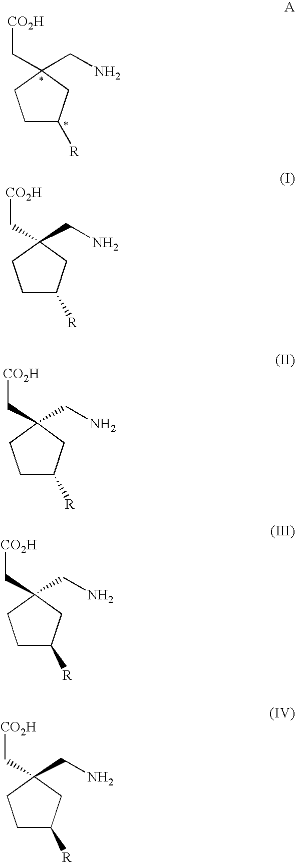

### BACKGROUND OF THE INVENTION

Compounds of formula 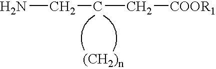
wherein R₁ is hydrogen or a lower alkyl radical and n is 4, 5, or 6 are known in U.S. Pat. No. 4,024,175 and its divisional U.S. Pat. No. 4,087,544. The uses disclosed are: protective effect against cramp induced by thiosemicarbazide; protective action against cardiazole cramp: the cerebral diseases, epilepsy, faintness attacks, hypokinesia, and cranial traumas: and improvement in cerebral functions. The compounds are useful in geriatric patients. The patents are hereby incorporated by reference.

U.S. Ser. No. 09/485,382 filed Feb. 8, 2000 teaches in part compounds of Formula I 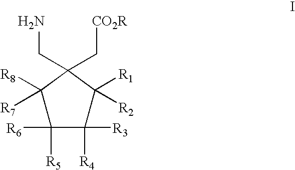
or a pharmaceutically acceptable salt thereof wherein R is hydrogen or a lower alkyl; and R₁ to R₈ are each independently selected from hydrogen, straight or branched alkyl of from 1 to 6 carbon atoms, phenyl, benzyl, fluorine, chlorine, bromine, hydroxy, hydroxymethyl, amino, aminomethyl, trifluoromethyl, —CO₂H, —CO₂R₁₅, —CH₂CO₂H, —CH₂CO₂R₁₅, —OR₁₅ wherein R₁₅ is a straight or branched alkyl of from 1 to 6 carbons, phenyl, or benzyl, and R₁ to R₈ are not simultaneously hydrogen. This patent application is hereby incorporated by reference.

U.S. Pat. No. 5,929,116 describes endothelin antagonists of formulas 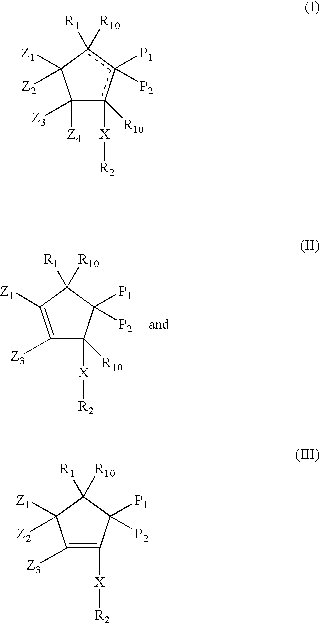
wherein
R₁ is —X(CH₂)ₙAr;
R₂ is Ar;
P₁ is —X(CH₂)ₙR₈;
P₂ is —X(CH₂)ₙR₈, or —XR₉Y;
R₃ and R₅ are independently hydrogen, R₁₁, OH, C₁₋₈ alkoxy, S(O)_{q}R₁₁, N(R₆)₂, Br, F, I, Cl, CF₃, NHCOR₆, —R₁₁CO₂R₇, —XR₉—Y, or —X(CH₂)ₙR₈ wherein each methylene group within —X(CH₂)ₙR₈ may be unsubstituted or substituted by one or two —(CH₂)ₙAr groups;
R₄ is hydrogen, R₁₁, OH, C₁₋₅ alkoxy, S(O)_{q}R₁₁, N(R₆)₂, —X(R₁₁), Br, F, I, Cl, or NHCOR₆ wherein the C₁₋₅ alkoxy may be unsubstituted or substituted by OH, methoxy, or halogen;
R₆ is independently hydrogen or C₁₋₄ alkyl;
R₇ is independently hydrogen, C₁₋₆ alkyl, or (CH₂)ₙAr;
R₈ is hydrogen, R₁₁, CO₂R₇, PO₃H₂, SO₂NR₇R₁₁, NR₇SO₂R₁₁, P(O)(OH)R₇, CN, —C(O)N(R₆)₂, tetrazole, or OR₆;
R₉ is C₁₋₁₀ alkyl, C₂₋₁₀ alkenyl, or phenyl, all of which may be unsubstituted or substituted by one or more OH, N(R₆)₂, COOH, >C═O, halogen, or XC₁₋₅ alkyl;
R₁₀ is R₃ or R₄;
R₁₁ is C₁₋₈ alkyl, C₂₋₈ alkenyl, C₂₋₈ alkynyl, all of which may be unsubstituted or substituted by one or more OH, CH₂OH, N(R₆)₂, or halogen;
X is (CH₂)ₙ, O, NR₆, or S(O)_{q};
Y is CH₃ or X(CH₂)ₙAr;
Ar is: 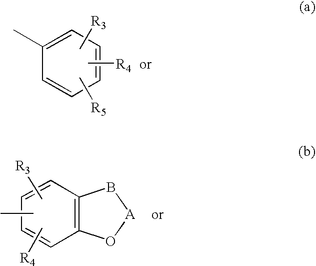
   naphthyl, indolyl, pyridyl, thienyl, oxazolidinyl, oxazolyl, thiazolyl, isothiazolyl, pyrazolyl, triazolyl, tetrazolyl, imidazolyl, imidazolidinyl, thiazolidinyl, isoxazolyl, oxadiazolyl, thiadiazolyl, morpholinyl, piperidinyl, piperazinyl, pyrrolyl, or pyrimidyl, all of which may be unsubstituted or substituted by one or more R₃ or R₄ groups;
A is >C═O, or [C(R₆)₂]m;
B is —CH₂— or —O—;
Z₁, Z₂, Z₃, and Z₄ are independently hydrogen, C₁₋₈ alkyl, C₂₋₈ alkenyl, C₂₋₈ alkynyl, OH, C₁₋₈ alkoxy, S(O)_{q}C₁₋₈ alkyl, N(R₆)₂, Br, F, I, Cl, NHCOR₆, —X(CH₂)ₙR₈, XR₉Y, phenyl, benzyl, or C₃₋₆ cycloalkyl wherein the C₁₋₈ alkyl, C₂₋₈ alkenyl, or C₂₋₈ alkynyl may be optionally substituted by COOH, OH, CO(CH₂)ₙCH₃, CO(CH₂)ₙCH₂N(R₆)₂, or halogen,
q is zero, one, or two;
n is an integer from 0 to 6;
m is 1, 2, or 3;
and the dotted line in Formula (I) indicates the optional presence of a double bond, or a pharmaceutically acceptable salt thereof; provided that
   when the optional double bond is present, there is only one R₁₀, there is no P₁, and P₂ is not NR₆R₉Y:
   X is not NR₆, and Z₃ is not OH or N(R₆)₂ in Formula (III);
Z₁ and Z₃ are not OH, N(R₆)₂, or Iodine in Formula (II);
when the optional double bond is present in Formula (I) and X—R₂ is attached to the double bond, X is not NR₆;
when the optional double bond is present in Formula (I) and R₁ is attached directly to the double bond, R₁ is not NR₆Ar;
when R₃, R₅, Z₁, Z₂, or Z₃ is X(CH₂)ₙR₈ and n is not zero, X is oxygen or NR₆ when R₈ is OR₆ or CO₂H.

Also included in the invention are pharmaceutically acceptable salts of the active compounds.

Most or all of the desired pharmacological activity of a compound comprised of two or more stereoisomers frequently resides in just one of the stereoisomers. The other stereoisomer(s) typically is inactive at best or exhibits undesirable side effects such as, for example, toxicity. Therefore where a compound is comprised of two or more stereoisomers, it is important, and sometimes mandatory, to develop a method of selectively preparing the beneficial stereoisomer in a form that is free from, or almost free from, contamination by the other inactive or harmful stereoisomer(s). However, usually it is very difficult to discover a method for the preparation of a beneficial stereoisomer in a form that is free from, or almost free from, contamination by the other inactive or harmful stereoisomer(s). Unexpectedly, we have invented novel preparations of certain important 3-substituted cyclopentyl-containing, amino acid analogs of gabapentin, a marketed anticonvulsant, which provide the desirable stereoisomers with a high degree of stereochemical purity.

None of the above teach the synthesis of the instant invention.

### SUMMARY OF THE INVENTION

The instant invention encompasses novel synthetic routes for the preparation of important 3-substituted cyclopentyl-based analogs of gabapentin and pharmaceutically acceptable salts thereof Gabapentin, marketed under the trade name Neurontin® for the treatment of seizure disorders, particularly epilepsy, provides well-known medical benefits to patients in need of such treatment. The instant invention encompasses novel synthetic routes for the preparation of 3-substituted cyclopentyl-based analogs of gabapentin and pharmaceutically acceptable salts thereof that enable the synthesis of each stereoisomer of these analogs with a high decree of stereochemical purity. These routes provide access to pure stereoisomers of Formulas I, II, III, and IV 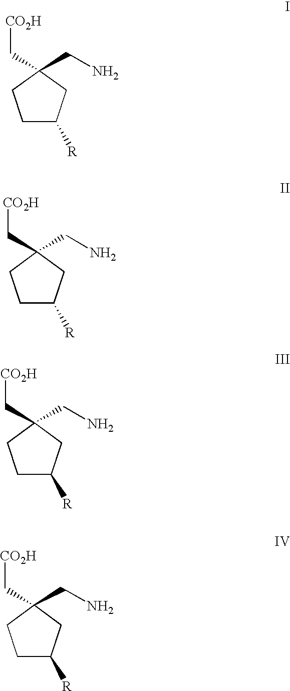
wherein R is C₁-C₁₀ alkyl or C₃-C₁₀ cycloalkyl.

Further, the invention encompasses the key intermediates of formulas (6) and (26). Still further, the invention provides novel synthetic routes for the preparation of compounds of formulas (6) and (26). The routes enable the synthesis of each stereoisomer of compounds of formulas (6) and (26) with a high degree of stereochemical purity. These routes provide access to pure stereoisomers of formulas (6) and (26) wherein R is C₁-C₁₀ alkyl or C₃-C₁₀ cycloalkyl.

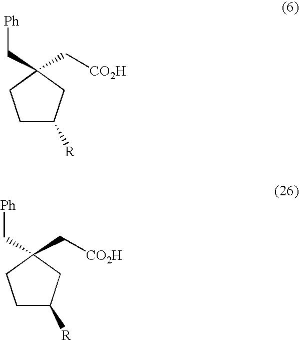

The invention provides a process for the preparation of a compound of Formula I 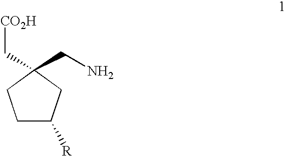
wherein R is C₁-C₁₀ alkyl or C₃-C₁₀ cycloalkyl, and pharmaceutically acceptable salts thereof, which comprises:
a) adding a cyanoacetate of formula (A) 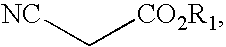
   wherein R₁ is alkyl or benzyl, to a mixture of a chiral cyclopentanone of formula 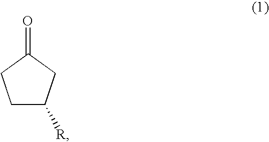
   a solvent, a carboxylic acid, and a Knoevenagel reaction catalyst, and stirring the mixture in the presence of a means of removing water to produce the alkene of formula 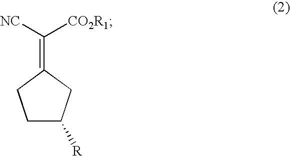
b) adding the product of Step a) above to a mixture of benzylmagnesium chloride, benzylmagnesium bromide, or benzylmagnesium iodide, in a solvent to produce the addition products of formulas 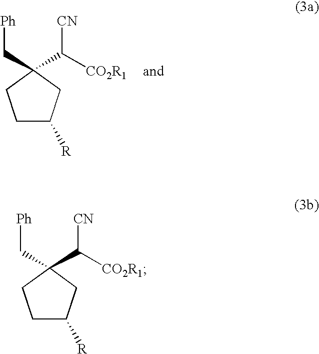
c) adding the products of Step b) above to a mixture of a base selected from potassium hydroxide, sodium hydroxide, lithium hydroxide, or cesium hydroxide in a solvent and stirring, and then acidifying to produce the carboxylic acids of formulas 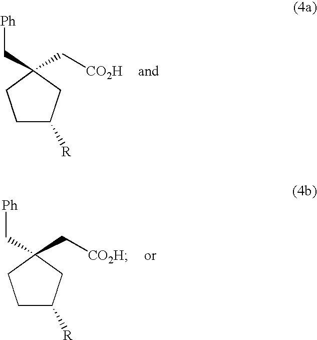
   adding the products of Step b) above to an acid mixture and stirring to produce the carboxylic acids of formulas 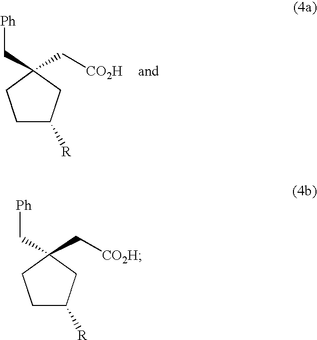
d) contacting the products of Step c) above with an amine in a solvent, and recrystallizing the salt so formed to produce the enriched diastereomer of formula 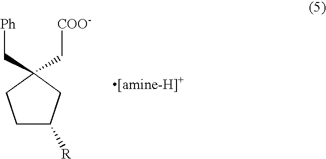
   as the amine salt;
e) converting the product of Step d) to a carboxylic acid of formula 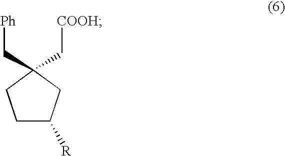
f) adding the product of Step e) to a mixture of iodomethane, a solvent, and a base, and stirring to produce the ester of formula 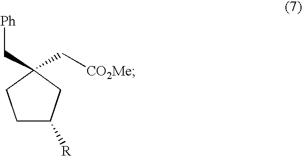
   or adding the product of Step e) to methanol and an acid to produce the ester of formula 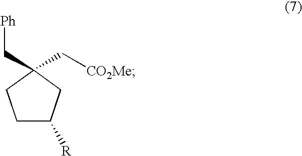
   or adding the product of Step e) above to trimethylsilyldiazo-methane and methanol in a solvent to produce the ester of formula 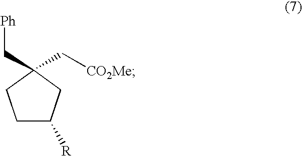
   or adding the product of Step e) to a solution of diazomethane or trimethylsilyl-diazomethane in a solvent to produce ester of formula 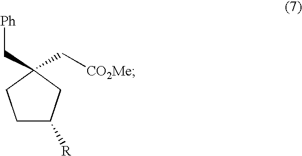
g) adding the product of Step f) to a mixture of carbon tetrachloride or ethyl acetate, and acetonitrile, water, sodium periodate, and ruthenium(III) chloride, and stirring to produce the carboxylic acid of formula 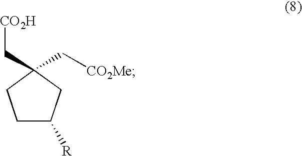
h) adding the product of Step g) to a mixture of a tertiary amine base, a solvent, and diphenylphosphoryl azide (DPPA), and stirring to produce the isocyanate of formula 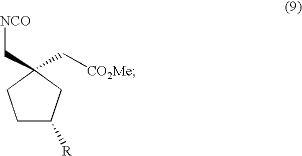
   or adding the product of
Step g) above to ethyl chloroformate or isobutyl chloroformate and a base in a solvent at a temperature of from −40° C. to 78° C., followed by adding a solution of sodium azide in water and tetrahydrofuran or acetone, followed by adding toluene or benzene, and refluxing to produce the isocyanate of formula 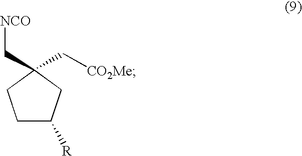
i) adding the product of Step h) to a mixture of a solvent and methanol, and stirring to produce the carbamate of formula 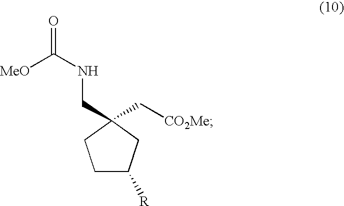
j) adding the product of Step i) to a mixture of a solvent and aqueous hydrochloric acid, and stirring to produce a compound of formula 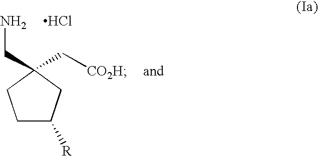
k) convening the product of Step j) to a compound of formula 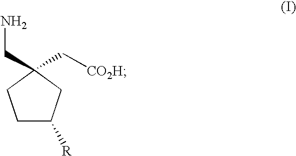
   and further convening, if desired, to a pharmaceutically acceptable salt by known means.

This process is outlined in Scheme 1.

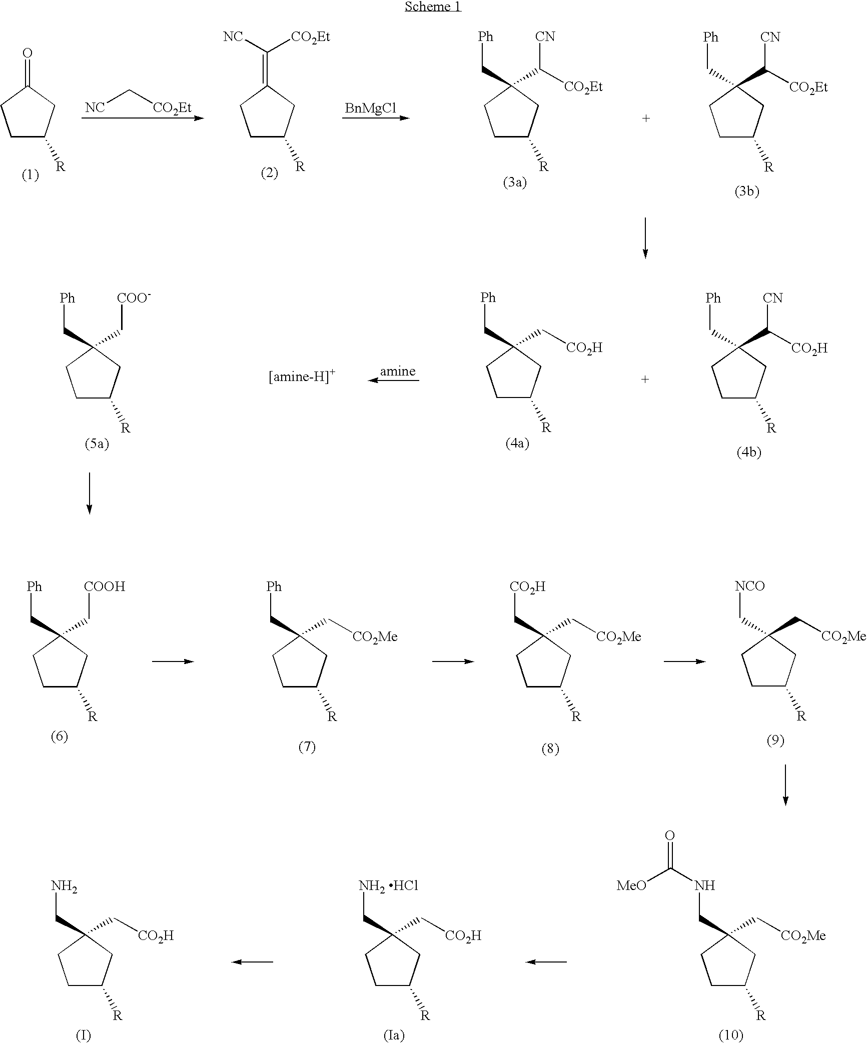

Preferred is a process for the preparation of a compound of Formula I wherein R is C₁-C₁₀ alkyl or C₃-C₁₀ cycloalkyl, and pharmaceutically acceptable salts thereof, which comprises:
a) adding a cyanoacetate of formula 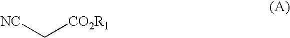
   wherein R₁ is selected from methyl, ethyl, n-propyl, iso-propyl, n-butyl iso-butyl, sec-butyl, tert-butyl, and benzyl to a mixture of a chiral cyclopentanone of formula 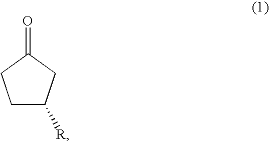
   a solvent selected from tetrahydrofuran, 1,4-dioxane, tert-butylmethylether, chloroform, dichloromethane, acetonitrile, ethyl ether, ethyl acetate, hexanes, N,N-dimethylformamide, dimethylsulfoxide, ethanol, tert-butanol, toluene, benzene, xylenes, and n-heptane, acetic acid, and a Knoevenagel reaction catalyst selected from β-alanine, ammonium acetate, and piperidine, and stirring the mixture in the presence of a means of removing water selected from azeotropic distillation, activated molecular sieves, anhydrous magnesium sulfate, anhydrous sodium sulfate, anhydrous sodium carbonate, anhydrous potassium carbonate, anhydrous cesium carbonate, trimethyl orthoformate, and triethyl orthoformate to produce the alkene of formula 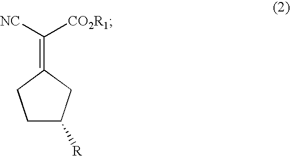
b) adding the product of Step a) above to a mixture of benzylmagnesium chloride, benzylmagnesium bromide, or benzylmagnesium iodide in a solvent selected from tetrahydrofuran, benzene, 1,4-dioxane, hexanes, n-heptane, toluene, diethyl ether, and tert-butyl methyl ether to produce the addition products of formulas 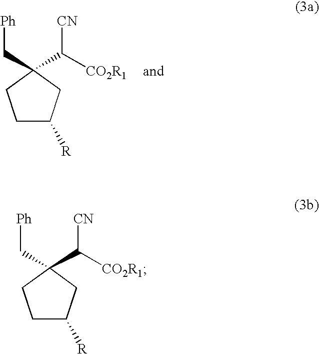
c) adding the products of Step b) above to a mixture of a base selected from potassium hydroxide, sodium hydroxide, lithium hydroxide, and cesium hydroxide in a solvent selected from ethylene glycol, 2-methoxyethyl ether, 1,4-dioxane, and diethylene glycol, and stirring the mixture, and then acidifying to produce the carboxylic acids of formulas 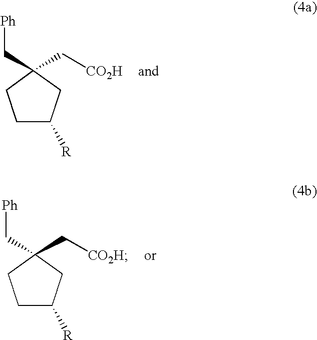
   adding the products of Step b) above to an acid mixture selected from 6-12 M HCl, 12 M H₂SO₄, 10%-48% wt/wt hydrobromic acid, and HBr in aqueous acetic acid, and stirring to produce the carboxylic acids of formulas 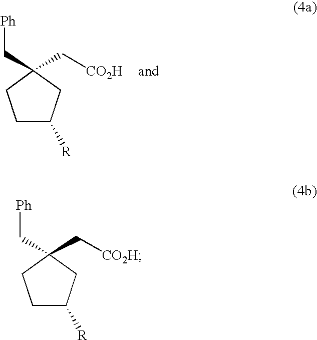
d) contacting the products of Step c) above with an amine selected from (S)-α-methyl-benzylamine, (R)-α-methyl-benzylamine, (R)-(+)-1-(naphthyl)ethylamine, (S)-(+)-1-(naphthyl)ethylamine, triethylamine, diisopropylethylamine, dicyclohexylamine, benzylamine, dibenzylamine, morpholine, N-methylmorpholine, piperidine, N-methylpiperidine, and pyridine in a solvent selected from N,N-dimethylformamide, chloroform, benzene, xylenes, hexanes, acetone, ethanol, methanol, iso-propanol, diethyl ether, dichloromethane, benzene, toluene, n-pentane, n-hexane, n-heptane, ethyl acetate, acetonitrile, tert-butyl methyl ether tetrahydrofuran, and 1,4-dioxane, and recrystallizing the salt so formed to produce the enriched diastereomer of formula 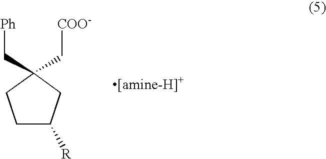
   as the amine salt;
e) adding the product of Step d) to a mixture selected from aqueous hydrochloric acid, aqueous sulfuric acid, aqueous acetic acid, hydrochloric acid dissolved in acetic acid, or hydrochloric acid dissolved in acetic acid to which water is added and stirring to produce the carboxylic acid of formula 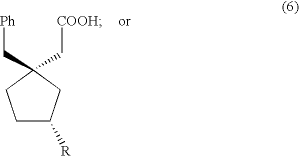
   partitioning the product of Step d) between a mixture of aqueous hydrochloric acid and a solvent selected from chloroform, dichloromethane, ethyl acetate, ethyl ether, tetrahydrofuran, 1,4-dioxane, toluene, and tert-butylmethylether, and drying and evaporating the organic layer to produce the carboxylic acid of formula 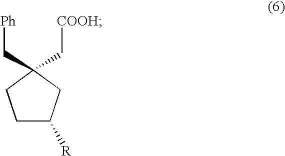
f) adding the product of Step e) above to a mixture of iodomethane, a solvent selected from dichloromethane, chloroform, tetrahydrofuran, toluene, and 1,4-dioxane, and a base selected from 1,8-diazabicyclo[5.4.0]undec-7-ene (DBU), diisopropylethylamine, triethylamine, and 1,5-diazabicyclo[4.3.0]non-5-ene (DBN), and stirring at a temperature of from −40° C. to 110° C. to produce the ester of formula 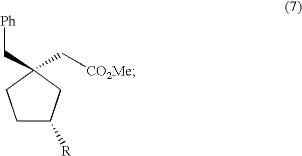
   or adding the product of Step e) above to a mixture of methanol and concentrated sulphuric acid, concentrated hydrochloric acid, or hydrogen chloride at a temperature of from 0° C. to 100° C. to produce the ester of formula 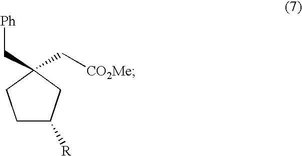
   or adding the product of Step e) above to trimethylsilyldiazomethane and methanol in benzene or toluene at a temperature of from −40° C. to 100° C. to produce the ester of formula 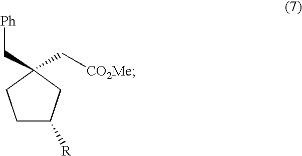
   or adding the product of Step e) above to diazomethane or trimethylsilyldiazomethane in a solvent selected from benzene, toluene, dichloromethane, and diethyl ether at a temperature of from −40° C. to 40° C. to give a compound of formula 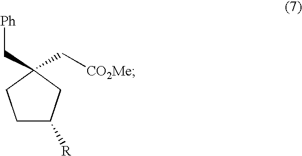
g) adding the product of Step f) to a mixture of carbon tetrachloride or ethyl acetate, and acetonitrile, water, sodium periodate, and ruthenium(III) chloride, and stirring at a temperature from −40° C. to 80° C. to produce the carboxylic acid of formula 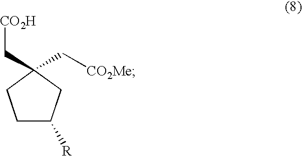
h) adding the product of Step g) above to a mixture of a base selected from triethylamine and diisopropylethylamine, a solvent selected from toluene, benzene, xylenes, tetrahydrofuran, diethyl ether and n-heptane, and diphenylphosphoryl azide (DPPA), and stirring at a temperature of from 0° C. to 150° C. to produce the isocyanate of formula 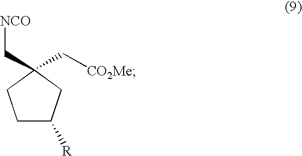
   or adding the product of Step g) above to ethyl chloroformate or isobutyl chloroformate, a base selected from triethylamine and diisopropylethylamine, and a solvent selected from tetrahydrofuran, acetone, and diethyl ether at a temperature of from −40° C. to 78° C., followed by adding a solution of sodium azide in water and tetrahydrofuran or acetone, followed by adding toluene or benzene, and refluxing to produce the isocyanate of formula 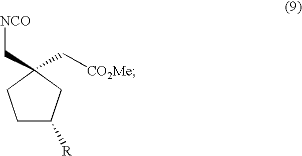
i) adding the product of Step h) to a mixture of a solvent selected from toluene, benzene, xylenes and n-heptane, and methanol, and stirring at a temperature from 0° C. to 150° C. to produce the carbamate of formula 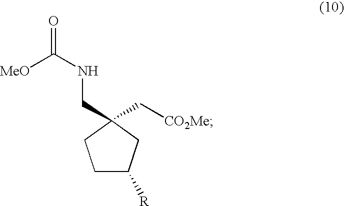
j) adding the product of Step i) to a mixture of a solvent selected from water, acetic acid, and 1,4-dioxane, and aqueous hydrochloric acid at a concentration of from 0.01 M to 12 M, and stirring at a temperature from 0° C. to 115° C. to produce a compound of formula Ia 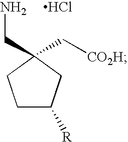
   and
k) converting the product of Step j) to a compound of Formula I 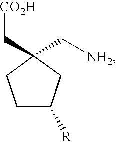
   and further converting if desired, to a pharmaceutically acceptable salt by known means.

More preferred is a process for the preparation of a compound of Formula I wherein R is C₁-C₁₀ alkyl or C₃-C₁₀ cycloalkyl, and pharmaceutically acceptable salts thereof, which comprises:
a) adding a cyanoacetate of formula 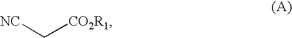
   wherein R₁ is ethyl, to a mixture of a chiral cyclopentanone of formula 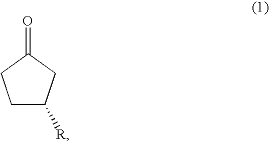
   toluene, acetic acid, and a Knoevenagel reaction catalyst which is ammonium acetate, and heating the mixture at reflux over a Dean-Stark trap to produce the alkene of formula 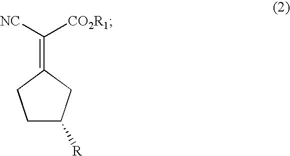
b) adding the product of Step a) above to a mixture of benzylmagnesium chloride in dry tetrahydrofuran at −100° C. to 25° C. to produce the addition products of formulas 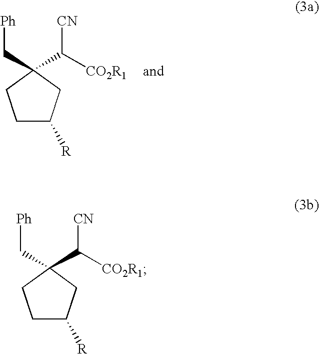
c) adding the products of Step b) above to a mixture of potassium hydroxide in ethylene glycol, and heating the mixture at 100° C. to 200° C., and then acidifying to produce the hydrolysis products of formulas 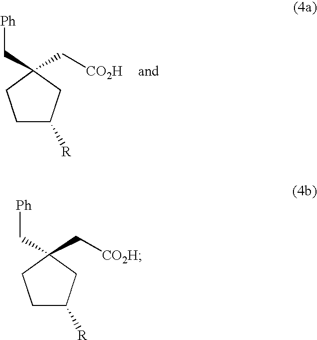
d) contacting the products of Step c) above with (S)-α-methyl-benzylamine in ethyl acetate, and recrystallizing the salt so formed from ethyl acetate to produce the enriched diastereomer of formula 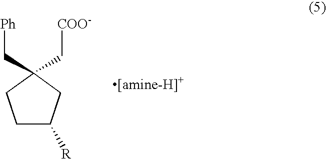
   as the (S)-α-methyl-benzylamine salt;
e) adding the product of Step d) to aqueous hydrochloric acid and stirring to produce the carboxylic acid of formula 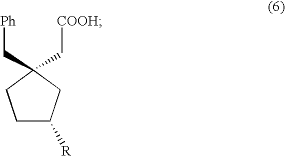
f) adding the product of Step e) to a mixture of iodomethane, dichloromethane and 1,8-diazabicyclo[5.4.0]undec-7-ene (DUB) and stirring to produce the ester of formula 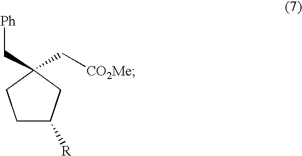
   or adding the product of Step e) to methanol and concentrated sulfuric acid to produce the ester of formula 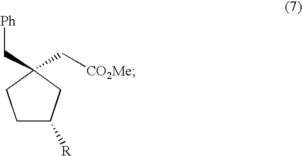
   or adding the product of Step e) to a solution of diazomethane or trimethylsilyl-diazomethane in dichloromethane to produce the ester of formula 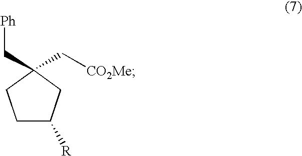
g) adding the product of Step f) to a mixture of carbon tetrachloride or ethyl acetate, and acetonitrile, water, sodium periodate, and ruthenium(III) chloride, and stirring to produce the carboxylic acid of formula 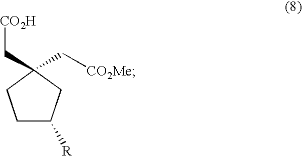
h) adding the product of Step g) to a mixture of triethylamine, toluene, and diphenylphosphoryl azide (DPPA), and refluxing to produce the isocyanate of formula 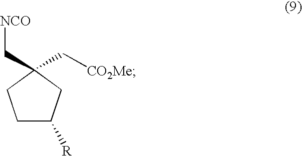
   or adding the product of Step g) above to ethyl chloroformate or isobutyl chloroformate and triethylamine in tetrahydrofuran at a temperature of from −40° C. to 78° C., followed by adding a solution of sodium azide in water and tetrahydrofuran, followed by adding toluene or benzene, and refluxing to produce ester of formula 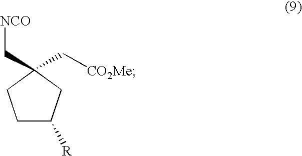
i) adding the product of Step h) to a mixture of methanol and toluene, and refluxing to produce the carbamate of formula 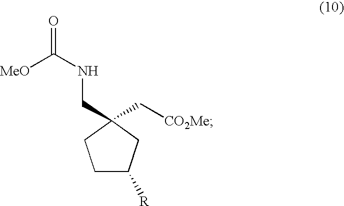
j) adding the product of Step i) to a mixture of 1,4-dioxane and aqueous hydrochloric acid at a concentration of 6 M, and stirring to produce a compound of formula Ia 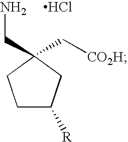
k) converting the product of Step j) to a compound of Formula I 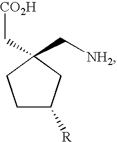
   and further converting, if desired, to a pharmaceutically acceptable salt by known means.

Also preferred is a process for the preparation of a compound of Formula I as described above, further characterized in that the intermediate product 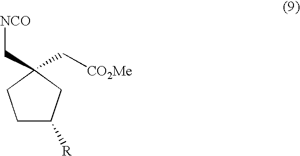
formed is reacted, without isolation, with methanol to produce the carbamate of formula

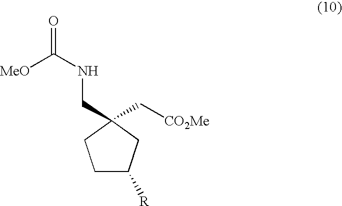

Further, the invention provides a process for the preparation of a compound of Formula II 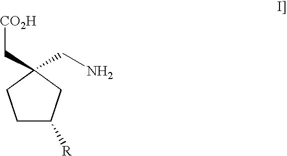
wherein R is C₁-C₁₀ alkyl or C₃-C₁₀ cycloalkyl, and pharmaceutically acceptable salts thereof, which comprises:
a) adding a cyanoacetate of formula 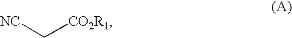
   wherein R₁ is alkyl or benzyl, to a mixture of a chiral cyclopentanone of formula 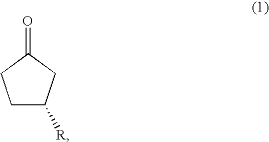
   a solvent, a carboxylic acid, and a Knoevenagel reaction catalyst, and stirring, the mixture in the presence of a means of removing water to produce the alkene of formula 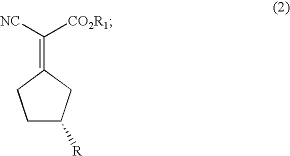
b) adding the product of Step a) above to a mixture of benzylmagnesium chloride, benzylmagnesium bromide, or benzylmagnesium iodide, in a solvent to produce the addition products of formulas 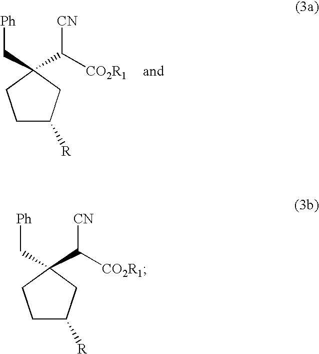
c) adding the products of Step b) above to a mixture of a base selected from potassium hydroxide, sodium hydroxide, lithium hydroxide, and cesium hydroxide and a solvent, and stirring, and then acidifying to produce the carboxylic acids of formulas 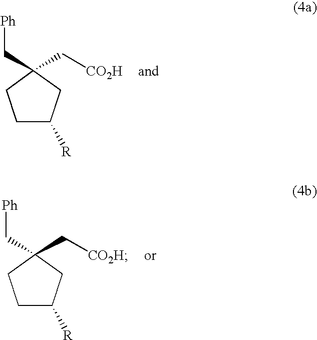
   adding the products of Step b) above to an acid mixture and stirring to produce the carboxylic acids of formulas 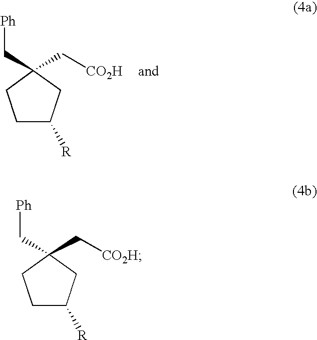
d) contacting the products of Step c) above with an amine in a solvent, and recrystallizing the salt so formed to produce the enriched diastereomer of formula 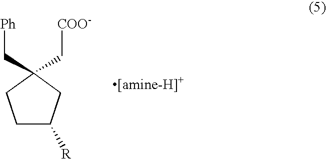
   as the amine salt; and
e) converting the product of Step d) to a carboxylic acid of formula 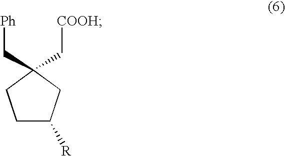
f) adding the product of Step e) to a mixture of a tertiary amine base, a solvent, and diphenylphosphoryl azide (DPPA), and stirring to produce the isocyanate of formula 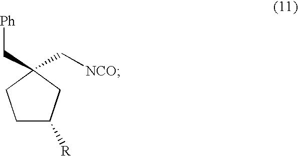
   or adding the product of Step
e) above to ethyl chloroformate or isobutyl chloroformate and a base in a solvent at a temperature of from −40° C. to 78° C. followed by adding a solution of sodium azide in water and tetrahydrofuran or acetone, followed by adding toluene or benzene, and refluxing to produce isocyanate of formula 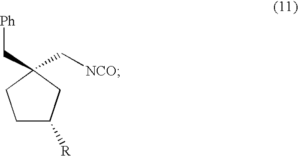
g) adding the product of Step f) to a mixture of a solvent and methanol, and stirring to produce the carbamate of formula 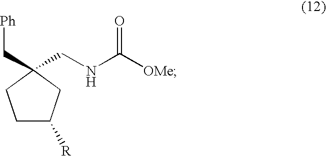
h) adding the product of Step g) to a mixture of carbon tetrachloride or ethyl acetate, and acetonitrile, water, sodium periodate, and ruthenium(III) chloride, and stirring to produce the carboxylic acid of formula 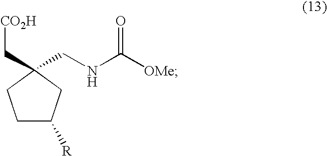
i) adding the product of Step h) to a mixture of a solvent and aqueous hydrochloric acid, and stirring to produce a compound of formula (IIa) 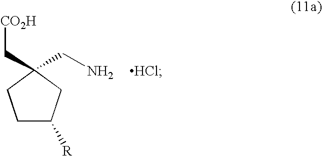
j) converting the product of Step i) to a compound of formula 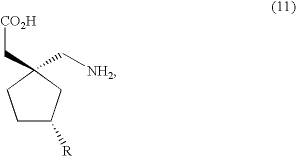
   and further converting, if desired, to a pharmaceutically acceptable salt by known means

This process is outlined below in Scheme 2.

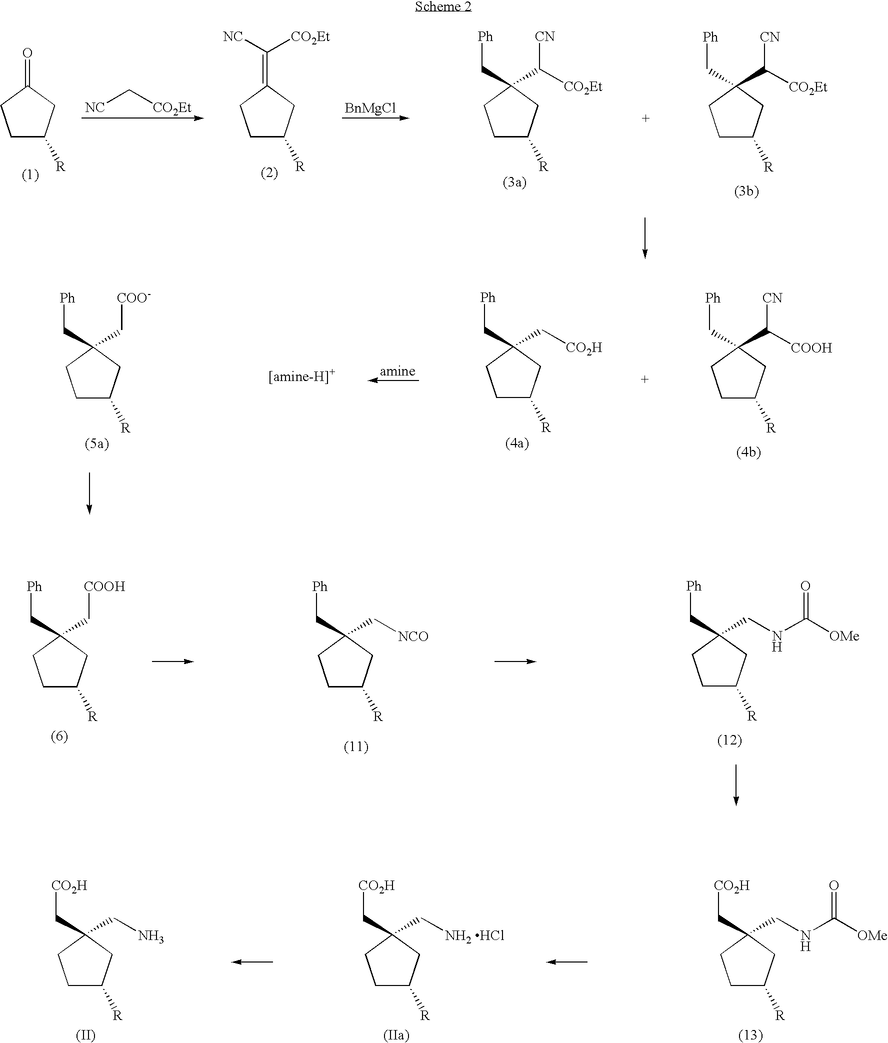

Preferred is a process for the preparation of a compound of Formula II wherein R is C₁-C₁₀ alkyl or C₃-C₁₀ cycloalkyl, and pharmaceutically acceptable salts thereof, which comprises:
a) adding a cyanoacetate of formula 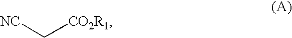
   wherein R₁ is selected from methyl, ethyl, n-propyl, iso-propyl, n-butyl, iso-butyl, sec-butyl, tert-butyl, and benzyl to a mixture of a chiral cyclopentanone of formula 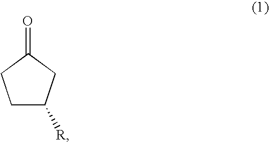
   a solvent selected from tetrahydrofuran, 1,4-dioxane, tert-butylmethylether, chloroform, dichloromethane, acetonitrile, ethyl ether, ethyl acetate, hexanes, N,N-dimethylformamide, dimethylsulfoxide, ethanol, tert-butanol, toluene, benzene, xylenes, and n-heptane, acetic acid, and a Knoevenagel reaction catalyst selected from β-alanine, ammonium acetate, and piperidine, and stirring the mixture in the presence of a means of removing water selected from azeotropic distillation, activated molecular sieves, anhydrous magnesium sulfate, anhydrous sodium sulfate, anhydrous sodium carbonate, anhydrous potassium carbonate, anhydrous cesium carbonate, trimethyl orthoformate, and triethyl orthoformate to produce the alkene of formula 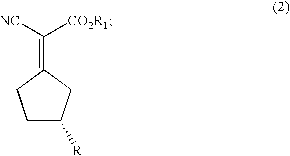
b) adding the product of Step a) above to a mixture of benzylmagnesium chloride, benzylmagnesium bromide, or benzylmagnesium iodide in a solvent selected from tetrahydrofuran, benzene, 1,4-dioxane, hexanes, n-heptane, toluene, diethyl ether, and tert-butyl methyl ether to produce the addition products of formulas 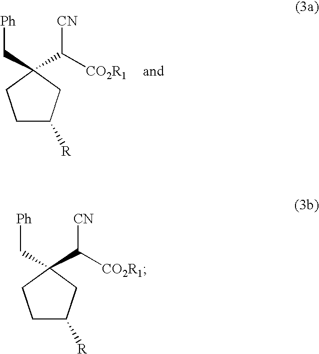
c) adding the products of Step b) above to a mixture of a base selected from potassium hydroxide, sodium hydroxide, lithium hydroxide, and cesium hydroxide in a solvent selected from ethylene glycol, 2-methoxyethyl ether, 1,4-dioxane, and diethylene glycol, and stirring the mixture and then acidifying to produce the carboxylic acids of formulas 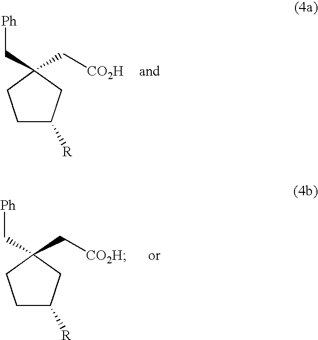
   adding the products of Step b) above to an acid mixture selected from 6-12 M HCl, 12 M H₂SO₄, 10%-48% wt/wt hvdrobromic acid, and HBr in aqueous acetic acid, and stirring to produce the carboxylic acids of formulas 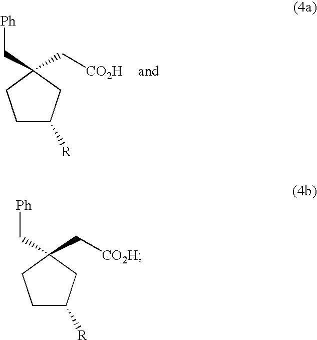
d) contacting the products of Step c) above with an amine selected from (S)-α-methyl-benzylamine, (R)-α-methyl-benzylamine, (R)-(+)-1-(naphthyl)ethylamine, (S)-(+)-1-(naphthyl)ethylamine, triethylamine, diisopropylethylamine, dicyclohexylamine, benzylamine, dibenzylamine, morpholine, N-methylmorpholine, piperidine, N-methylpiperidine, and pyridine in a solvent selected from N,N-dimethylformamide, chloroform, benzene, xylenes, hexanes, acetone, ethanol, methanol, iso-propanol, diethyl ether, dichloromethane, benzene, toluene, n-pentane, n-hexane, n-heptane, ethyl acetate, acetonitrile, tert-butyl methyl ether, tetrahydrofuran, and 1,4-dioxane, and recrystallizing the salt so formed to produce the enriched diastereomer of formula 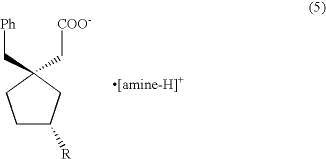
   as the amine salt:
e) adding the product of Step d) to a mixture selected from aqueous hydrochloric acid, aqueous sulfuric acid, aqueous acetic acid, hydrochloric acid dissolved in acetic acid, and hydrochloric acid dissolved in acetic acid and water, and stirring to produce the carboxylic acid of formula 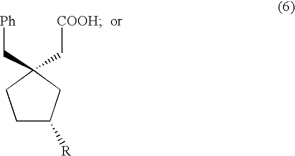
   partitioning the product of Step d) between a mixture of aqueous hydrochloric acid and a solvent selected from chloroform, dichloromethane, ethyl acetate, ethyl ether, tetrahydrofuran, 1,4-dioxane, toluene, and tert-butylmethylether, and drying and evaporating the organic layer to produce the carboxylic acid of formula 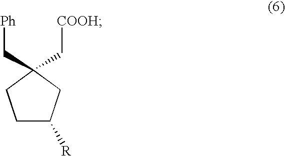
f) adding the product of Step e) above to a mixture of a base selected from triethylamine and diisopropylethylamine, a solvent selected from toluene, benzene, xylenes, tetrahydrofuran, diethyl ether and n-heptane, and diphenylphosphoryl azide (DPPA), and stirring at a temperature of from 0° C. to 150° C. to produce the isocyanate of formula 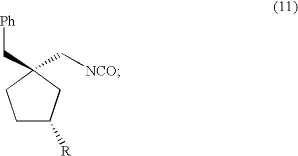
   or adding the product of Step e) above to ethyl chloroformate or isobutyl chloroformate and a base selected from triethylamine and diisopropylethylamine, and a solvent selected from tetrahydrofuran acetone, and diethyl ether at a temperature of from −40° C. to 78° C., followed by adding a solution of sodium azide in water and tetrahydrofuran or acetone, followed by adding toluene or benzene, and refluxing to produce the isocyanate of formula 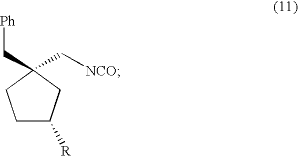
g) adding the product of Step f) to a solvent selected from toluene, benzene, xylenes, and n-heptane, and methanol, and stirring at a temperature from 0° C. to 150° C. to produce the carbamate of formula 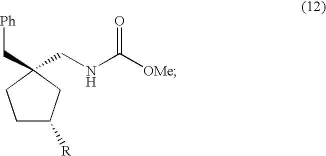
h) adding the product of Step g) to a mixture of carbon tetrachloride or ethyl acetate, and acetonitrile, water, sodium periodate, and ruthenium(III) chloride, and stirring at a temperature from −40° C. to 80° C. to produce the carboxylic acid of formula 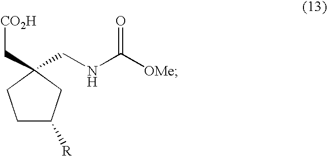
i) adding the product of Step h) to a mixture of a solvent selected from water, acetic acid, and 1,4-dioxane, and aqueous hydrochloric acid at a concentration of from 0.01 M to 12 M, and stirring at a temperature from 0° C. to 115° C. to produce a compound of formula IIa 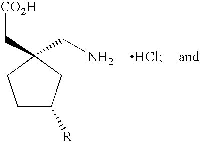
j) converting the product of Step i) to a compound of Formula II 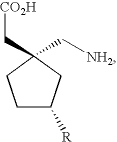
   and further converting, if desired, to a pharmaceutically acceptable salt by known means.

More preferred is a process for the preparation of a compound of Formula II wherein R is C₁-C₁₀ alkyl or C₃-C₁₀ cycloalkyl, and pharmaceutically acceptable salts thereof, which comprises:
a) adding a cyanoacetate of formula 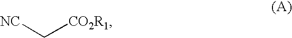
   wherein R₁ is ethyl, to a mixture of a chiral cyclopentanone of formula 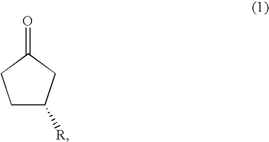
   toluene, acetic acid, and a Knoevenagel reaction catalyst which is ammonium acetate, and heating the mixture at reflux over a Dean-Stark trap to produce the alkene of formula 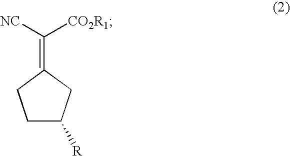
b) adding the product of Step a) above to a mixture of benzylmagnesium chloride in dry tetrahydrofuran at −100° C. to 25° C. to produce the addition products of formulas 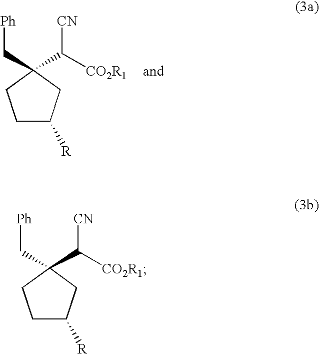
c) adding the products of Step b) above to a mixture of potassium hydroxide in ethylene glycol, and heating the mixture at 100° C. to 200° C., and then acidifying to produce the hydrolysis products of formulas 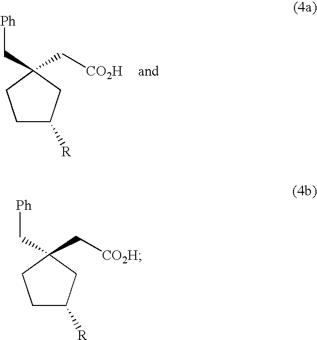
d) contacting the products of Step c) above with (S)-α-methyl-benzylamine in ethyl acetate, and recrystallizing the salt so formed from ethyl acetate to produce the enriched diastereomer of formula 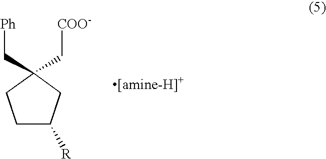
   as the (S)-α-methyl-benzylamine salt;
e) adding the product of Step d) to aqueous hydrochloric acid and stirring to produce the carboxylic acid of formula 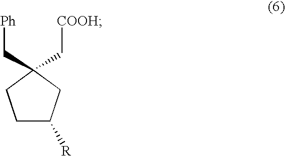
f) adding the product of Step e) to a mixture of triethylamine, toluene, and diphenylphosphoryl azide (DPPA), and refluxing to produce the isocyanate of formula 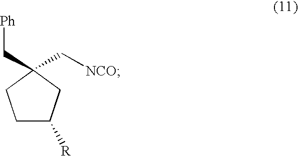
   or adding the product of Step e) above to ethyl chloroformate or isobutyl chloroformate and triethylamine in tetrahydrofuran at a temperature of from −40° C. to 78° C., followed by adding a solution of sodium azide in water and tetrahydrofuran or acetone, followed by adding toluene or benzene, and refluxing to produce isocyanate of formula 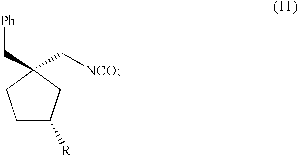
g) adding the product of Step f) to a mixture of methanol and toluene, and refluxing to produce the carbamate of formula 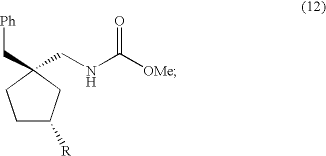
h) adding the product of Step g) to a mixture of carbon tetrachloride or ethyl acetate, and acetonitrile, water, sodium periodate, and ruthenium(III) chloride, and stirring to produce the carboxylic acid of formula 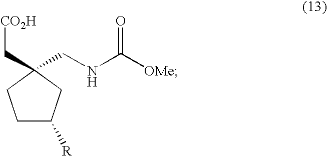
i) adding the product of Step h) to a mixture of 1,4-dioxane and aqueous hydrochloric acid at a concentration of 6 M, and stirring to produce a compound of formula IIa 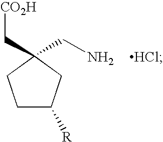
j) converting the product of Step i) to a compound of Formula II 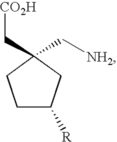
   and further converting, if desired, to a pharmaceutically acceptable salt by known means.

Also preferred is a process for the preparation of a compound of Formula II as described above, further characterized in that the intermediate product 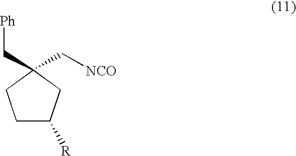
formed is further reacted, without isolation, with methanol to produce the carbamate of formula

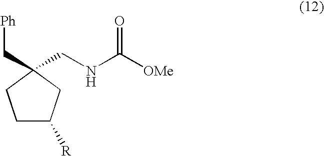

Still further, the invention provides a process for the preparation of a compound of Formula II 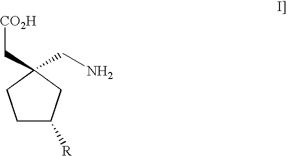
wherein R is C₁-C₁₀ alkyl or C₃-C₁₀ cycloalkyl, and pharmaceutically acceptable salts thereof, which comprises:
a) adding a cyanoacetate of formula 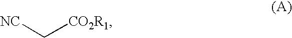
   wherein R₁ is alkyl or benzyl, to a mixture of a chiral cyclopentanone of formula 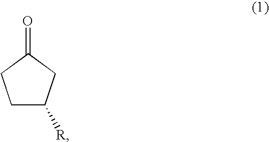
   a solvent, a carboxylic acid, and a Knoevenagel reaction catalyst, and stirring the mixture in the presence of a means of removing water to produce the alkene of formula 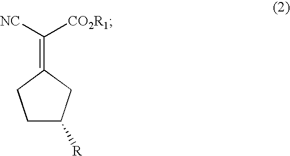
b) adding the product of Step a) above to a mixture of benzylmagnesium chloride, benzylmagnesium bromide, or benzylmagnesium iodide, in a solvent to produce the addition products of formulas 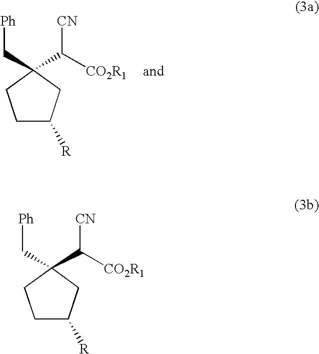
c) adding the products of Step b) above to a mixture of a base selected from potassium hydroxide, sodium hydroxide, lithium hydroxide, and cesium hydroxide, in a solvent, and stirring, and then acidifying to produce the carboxylic acids of formulas 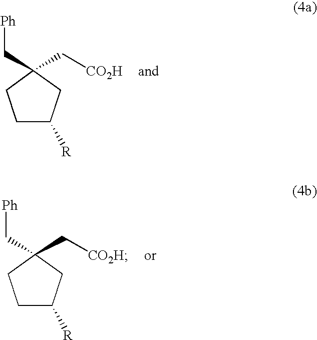
   adding the products of Step b) above to an acid mixture and stirring to produce the carboxylic acids of formulas 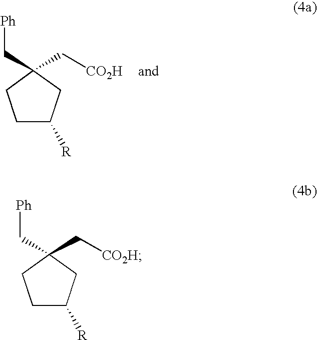
d) contacting the products of Step c) above with an amine in a solvent., and recrystallizing the salt so formed to produce the enriched diastereomer of formula 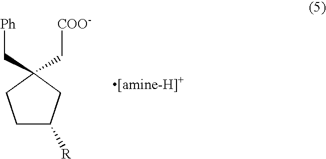
   as the amine salt.
e) converting the product of Step d) to a carboxylic acid of formula 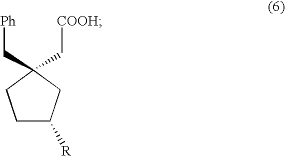
f) adding oxalyl chloride to a mixture of the product of Step e), a solvent, and N,N-dimethylformamide (DMF), and stirring to produce the acid chloride of formula 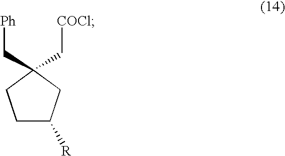
g) adding the product of Step f) to a mixture of tert-butyl alcohol, a solvent, and a tertiary amine base, and stirring to produce the ester of formula 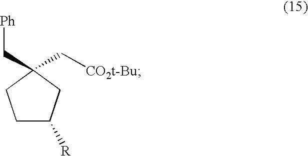
h) adding the product of Step g) to a mixture of carbon tetrachloride or ethyl acetate, and acetonitrile, water, sodium periodate, and ruthenium(III) chloride, and stirring to produce the carboxylic acid of formula 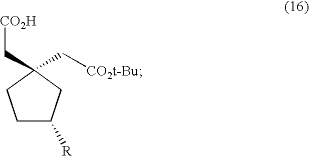
i) adding the product of Step h) to a mixture of a solvent, methanol, and (trimethylsilyl)diazomethane, and stirring to produce the bis ester of formula 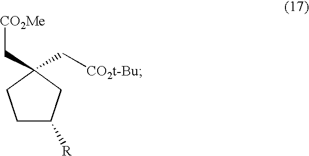
   or adding the product of Step h) to a mixture of iodomethane, a solvent, and a base, and stirring to produce the bis ester of formula 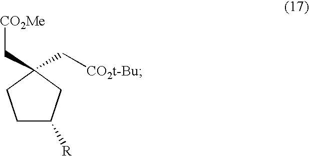
j) adding an acid to a mixture of the product from Step i) and a solvent, and stirring to produce the carboxylic acid of formula 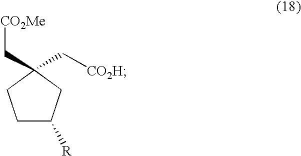
k) adding the product of Step j) to a mixture of a tertiary amine base, a solvent, and diphenylphosphoryl azide (DPPA) is added, and stirring to produce the isocyanate of formula 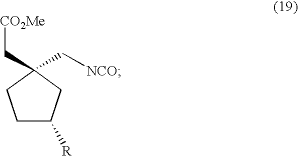
   or adding the product of Step j) above to ethyl chloroformate or isobutyl chloroformate and a base in a solvent at a temperature of from −40° C. to 78° C., followed by adding a solution of sodium azide in water and tetrahydrofuran or acetone, followed by adding toluene or benzene, and refluxing to produce isocyanate of formula 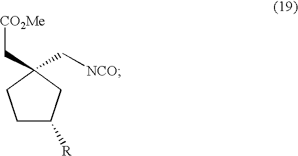
l) adding the product of Step k) to a mixture of a solvent and methanol, and stirring to produce the carbamate of formula 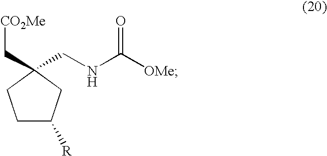
m) adding the product of Step l) to a mixture of a solvent and aqueous hydrochloric acid is added, and stirring to produce a compound of formula 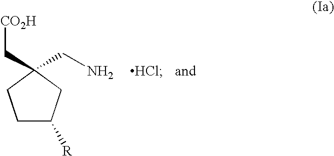
n) converting the product of Step m) to a compound of formula 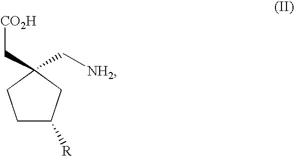
   and further converting, if desired, to a pharmaceutically acceptable salt by known means.

This process is outlined in Scheme 3.

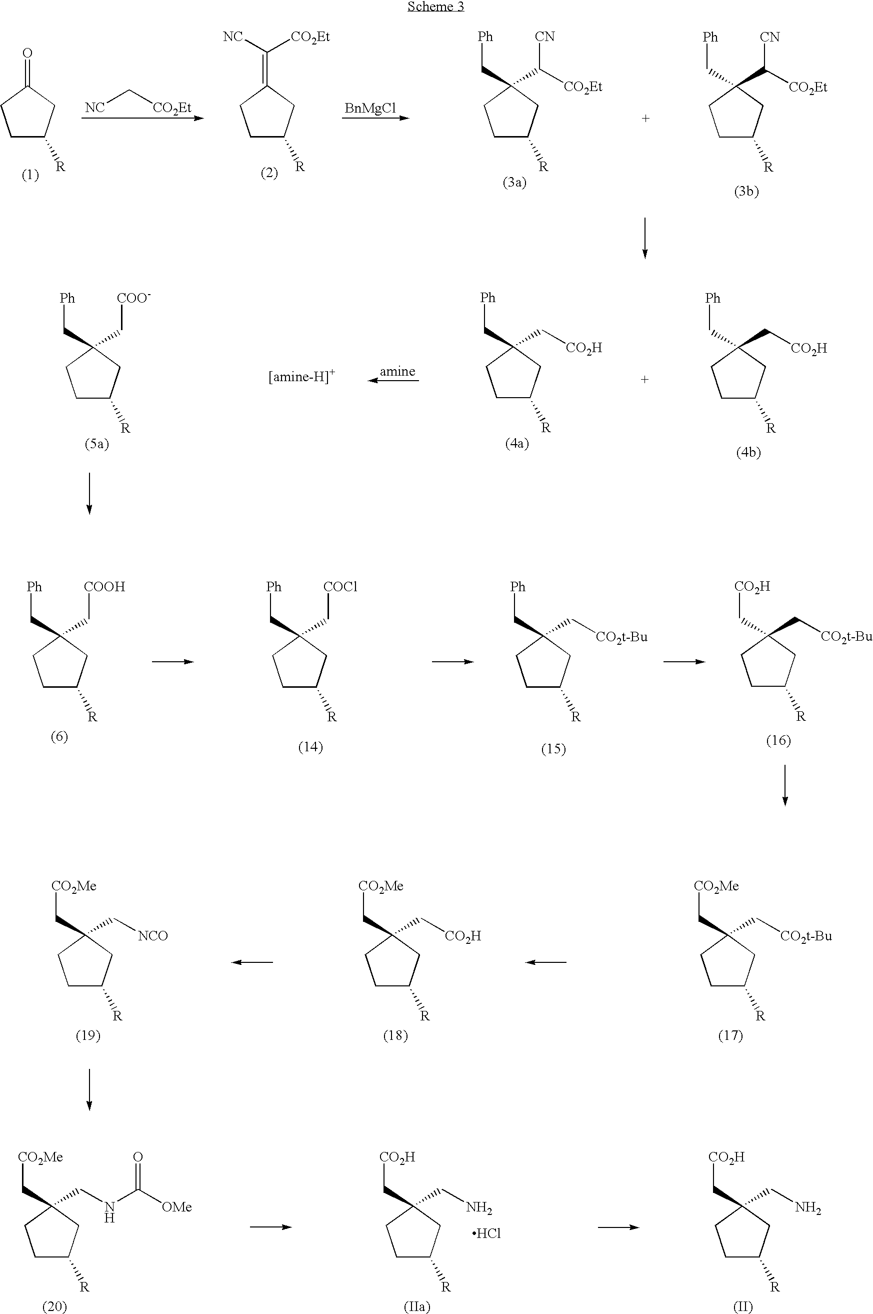

Preferred is a process for the preparation of a compound of Formula II wherein R is C₁-C₁₀ alkyl or C₃-C₁₀ cycloalkyl, and pharmaceutically acceptable salts thereof, which comprises:
a) adding a cyanoacetate of formula 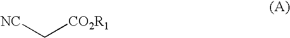
   wherein R₁ is selected from methyl, ethyl, n-propyl, iso-propyl, n-butyl, iso-butyl, sec-butyl, tert-butyl, and benzyl to a mixture of a chiral cyclopentanone of formula 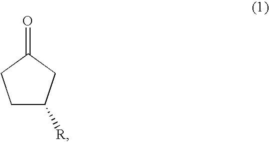
   a solvent selected from tetrahydrofuran, 1,4-dioxane, tert-butylmethylether, chloroform, dichloromethane, acetonitrile, ethyl ether, ethyl acetate, hexanes, N,N-dimethylformamide, dimethylsulfoxide, ethanol, tert-butanol, toluene, benzene, xylenes, and n-heptane, acetic acid, and a Knoevenaeel reaction catalyst selected from β-alanine, ammonium acetate, and piperidine, and stirring the mixture in the presence of a means of removing water selected from azeotropic distillation, activated molecular sieves, anhydrous magnesium sulfate, anhydrous sodium sulfate, anhydrous sodium carbonate, anhydrous potassium carbonate, anhydrous cesium carbonate, trimethyl orthoformate, and triethyl orthoformate to produce the alkene of formula 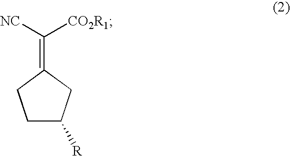
b) adding the product of Step a) above to a mixture of benzylmagnesium chloride, benzylmagnesium bromide, or benzylmagnesium iodide in a solvent selected from tetrahydrofuran, benzene, 1,4-dioxane, hexanes, n-heptane, toluene, diethyl ether, and tert-butyl methyl ether to produce the addition products of formulas 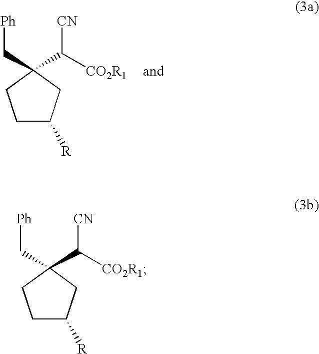
c) adding the products of Step b) above to a mixture of a base selected from potassium hydroxide, sodium hydroxide, lithium hydroxide, and cesium hydroxide in a solvent selected from ethylene glycol, 2-methoxyethyl ether, 1,4-dioxane, and diethylene glycol, and stirring the mixture and then acidifying to produce the carboxylic acids of formulas 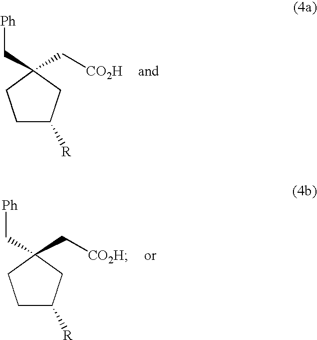
   adding the products of Step b) above to an acid mixture selected from 6-12 M HCl, 12 M H₂SO₄, 10%-48% wt/wt hvdrobromic acid, and HBr in aqueous acetic acid, and stirring to produce the carboxylic acids of formulas 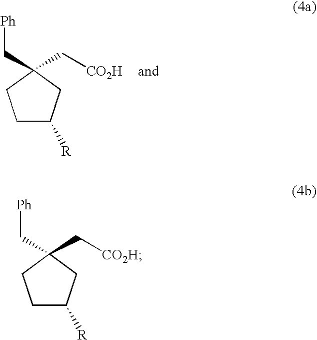
d) contacting the products of Step c) above with an amine selected from (S)-α-methyl-benzylamine, (R)-α-methyl-benzylamine, (R)-(+)-1-(naphthyl)ethylamine, (S)-(+)-1-(naphthyl)ethylamine, triethylamine, diisopropylethylamine, dicyclohexylamine, benzylamine, dibenzylamine, morpholine, N-methylmorpholine, piperidine, N-methylpiperidine, and pyridine in a solvent selected from N,N-dimethylformamide, chloroform, benzene, xylenes, hexanes, acetone, ethanol, methanol, iso-propanol, diethyl ether, dichloromethane, benzene, toluene, n-pentane, n-hexane, n-heptane, ethyl acetate, acetonitrile, tert-butyl methyl ether, tetrahydrofuran, and 1,4-dioxane, and recrystallizing the salt so formed to produce the enriched diastereomer of formula 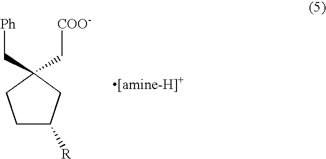
   as the amine salt,
e) adding the product of Step d) to a mixture selected from aqueous hydrochloric acid, aqueous sulfuric acid, aqueous acetic acid, hydrochloric acid dissolved in acetic acid, or hydrochloric acid dissolved in acetic acid and water, and stirring to produce the carboxylic acid of formula 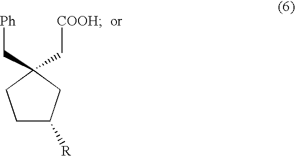
   partitioning the product of Step d) between a mixture of aqueous hydrochloric acid and a solvent selected from chloroform, dichloromethane, ethyl acetate, ethyl ether, tetrahydrofuran, 1,4-dioxane, toluene, and tert-butylmethylether, and drying and evaporating the organic layer to produce the carboxylic acid of formula 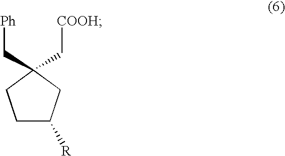
f) adding oxalyl chloride to a mixture of the product of Step e), a solvent selected from dichloromethane, chloroform, ethyl ether, toluene, and tert-butyl methyl ether, and 0.01 to 10 mole percent of N,N-dimethylformamide (DMF), and stirring at a temperature from −40° C. to 110° C. to produce the acid chloride of formula 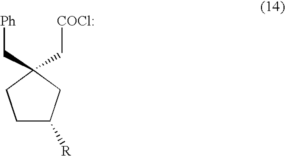
g) adding the product of Step f) to a mixture of tert-butyl alcohol, a solvent selected from dichloromethane, chloroform, ethyl ether, toluene, and tert-butyl methyl ether, and N,N-diisopropylethylamine (DIPEA) or triethylamine, and stirring at a temperature from −40° C. to 110° C. to produce the ester of formula 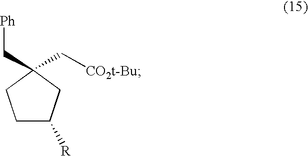
h) adding the product of Step g) to a mixture of carbon tetrachloride or ethyl acetate, and acetonitrile, water, sodium periodate, and ruthenium(III) chloride, and stirring at a temperature from −40° C. to 80° C. to produce the carboxylic acid of formula 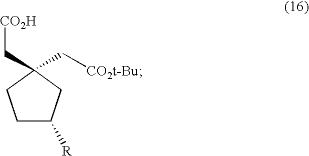
i) adding the product of Step h) to a solvent selected from toluene, benzene, xylenes, and n-heptane, methanol, and (trimethylsilyl)diazomethane, and stirring at a temperature from 0° C. to 150° C. to produce the bis ester of formula 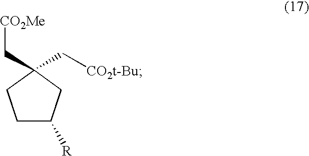
   or adding the product of Step h) to a mixture of iodomethane, a solvent selected from dichloromethane, chloroform, tetrahydrofuran, toluene and 1,4-dioxane, and a base selected from 1,8-diazabicyclo[5.4.0]undec-7-ene (DBU), diisopropylethylamine, triethylamine, or 1,5-diazabicyclo[4.3.0]non-5-ene (DBN), and stirring at a temperature of from −40° C. to 110° C. to produce the bis ester of formula 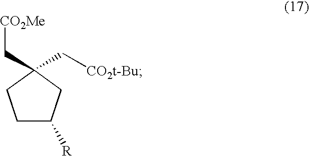
j) adding hydrochloric acid or trifluoroacetic acid (TFA) to a mixture of the product from Step i) and a solvent selected from dichloromethane, chloroform, 1,4-dioxane, tetrahydrofuran, ethyl ether, and tert-butyl methyl ether, and stirring at a temperature from −40° C. to 110° C. to produce the carboxylic acid of formula 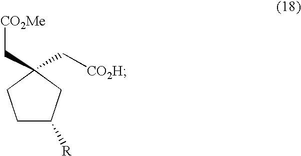
k) adding the product of Step j) to a mixture of a base selected from triethylamine and diisopropylethylamine, a solvent selected from toluene, benzene, xylenes, and n-heptane, and diphenylphosphoryl azide (DPPA), and stirring at a temperature from 0° C. to 150° C. to produce the isocyanate of formula 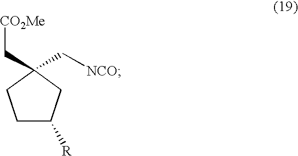
   or adding the product of Step j) above to ethyl chloroformate or isobutyl chloroformate, a base selected from triethylamine and diisopropylethylamine, and a solvent selected from tetrahydrofuran, acetone, and diethyl ether at a temperature of from −40° C. to 78° C., followed by adding a solution of sodium azide in water and tetrahydrofuran or acetone, followed by adding toluene or benzene, and refluxing to produce isocyanate of formula 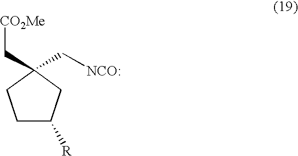
l) adding the product of Step k) to a mixture of a solvent selected from toluene, benzene, xylenes, and n-heptane, and methanol, and stirring at a temperature from 0° C. to 150° C. to produce the carbamate of formula 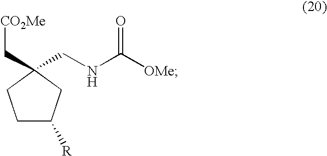
m) adding the product of Step l) to a mixture of a solvent selected from water, acetic acid, and 1,4-dioxane, and aqueous hydrochloric acid at a concentration of from 0.01 M to 12 M, and stirring at a temperature from 0° C. to 115° C. to produce a compound of formula IIa 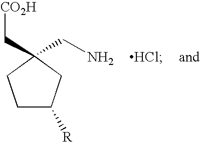
n) converting the product of Step m) to a compound of Formula II 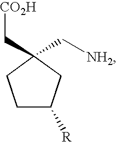
   and further converting, if desired, to a pharmaceutically acceptable salt by known means.

More preferred is a process for the preparation of a compound of Formula II wherein R is C₁-C₁₀ alkyl or C₃-C₁₀ cycloalkyl, and pharmaceutically acceptable salts thereof, which comprises:
a) adding a cyanoacetate of formula 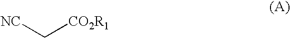
   wherein R₁ is ethyl, to a mixture of a chiral cyclopentanone of formula 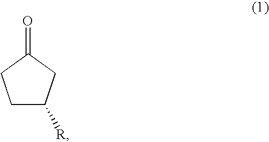
   toluene, acetic acid, and a Knoevenagel reaction catalyst which is ammonium acetate, and heating the mixture at reflux over a Dean-Stark trap to produce the alkene of formula 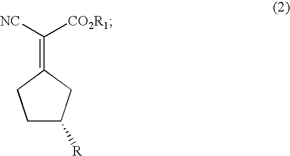
b) adding the product of Step a) above to a mixture of benzylmagnesium chloride in dry tetrahydrofuran at −100° C. to 25° C. to produce the addition products of formulas 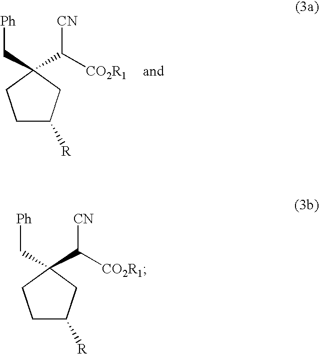
c) adding the products of Step b) above to a mixture of potassium hydroxide in ethylene glycol and heating the mixture at 100° C. to 200° C., and then acidifying to produce the hydrolysis products of formulas 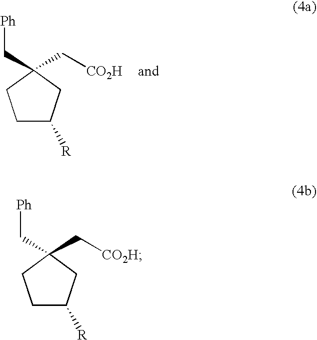
d) contacting the products of Step c) above with (S)-α-methyl-benzylamine in ethyl acetate, and recrystallizing the salt so formed from ethyl acetate to produce the enriched diastereomer of formula 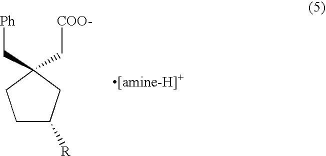
   as the (S)-α-methyl-benzylamine salt;
e) adding the product of Step d) to aqueous hydrochloric acid and stirring to produce the carboxylic acid of formula 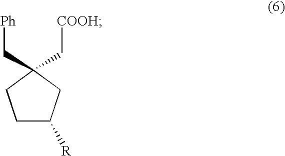
f) adding oxalyl chloride to a mixture of the product of Step e), dichloromethane, and a catalytic amount of N,N-dimethylformamide (DMF), and stirring to produce the acid chloride of formula 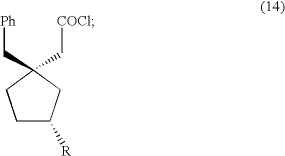
g) adding the product of Step f) to a mixture of tert-butyl alcohol, dichloromethane, and N,N-diisopropylethylamine (DIPEA), and stirring to produce the ester of formula 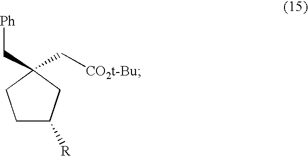
h) adding the product of Step g) to a mixture of carbon tetrachloride or ethyl acetate, and acetonitrile, water, sodium periodate, and ruthenium(III) chloride, and stirring to produce the carboxylic acid of formula 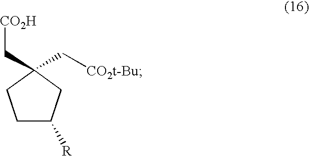
i) adding the product of Step h) to a mixture of methanol, toluene, and (trimethylsilyl)diazomethane, and stirring to produce the bis ester of formula 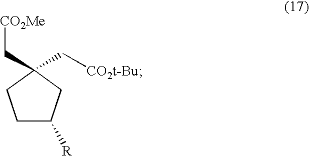
   or adding the product of Step h) to a mixture of iodomethane, dichloromethane, triethylamine, and stirring to produce the bis ester of formula 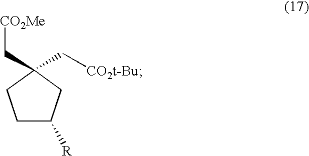
j) adding hydrochloric acid or trifluoroacetic acid (TFA) to a mixture of the product from Step i) and dichloromethane, and stirring to produce the carboxylic acid of formula 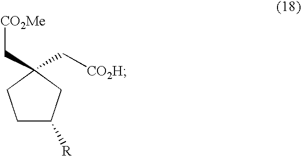
k) adding the product of Step j) to a mixture of triethylamine, toluene, and diphenylphosphoryl azide (DPPA), and refluxing to produce the isocyanate of formula 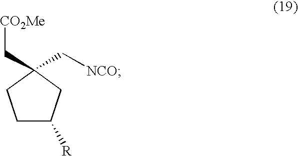
   or adding the product of Step j) above to ethyl chloroformate or isobutyl chloroformate, triethylamine, and tetrahydrofuran at a temperature of from −40° C. to 78° C., followed by adding a solution of sodium azide in water and tetrahydrofuran or acetone, followed by adding toluene or benzene, and refluxing to produce isocyanate of formula 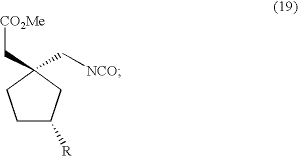
l) adding the product of Step k) to a mixture of methanol and toluene, and refluxing to produce the carbamate of formula 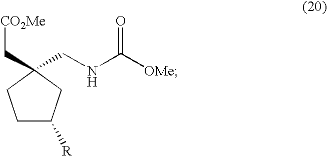
m) adding the product of Step l) to a mixture of 1,4-dioxane and aqueous hydrochloric acid at a concentration of 6 M, and stirring to produce a compound of formula IIa 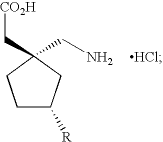
n) converting the product of Step m) to a compound of Formula II 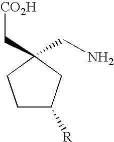
   and further converting, if desired, to a pharmaceutically acceptable salt by known means.

Also preferred is a process for the preparation of a compound of Formula II, further characterized in that the intermediate product 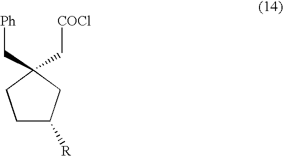
formed is further reacted, without isolation, with tert-butyl alcohol to produce the ester of formula

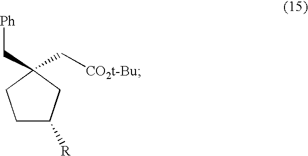

Also preferred is a process for the preparation of a compound of Formula II, further characterized in that the intermediate product 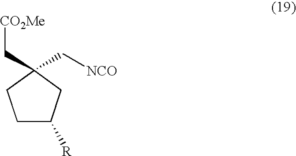
formed is further reacted, without isolation, with methanol to produce the carbamate of formula

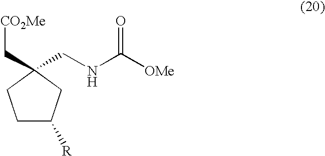

Also preferred is a process for the preparation of a compound of Formula II, further characterized in that the intermediate product 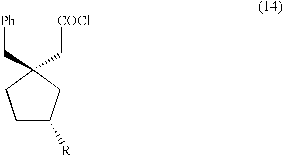
formed is further reacted without isolation, with tert-butyl alcohol to produce the ester of Formula 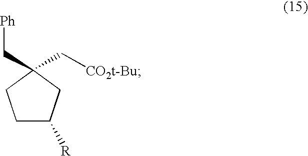
and the intermediate product 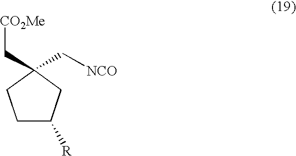
formed is further reacted, without isolation, with methanol to produce the carbamate of formula

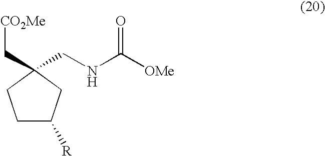

Further, the invention provides a process for the preparation of a compound of Formula III 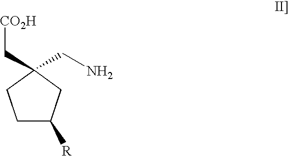
wherein R is C₁-C₁₀ alkyl or C₃-C₁₀ cycloalkyl, and pharmaceutically acceptable salts thereof, which comprises:
a) adding a cyanoacetate of formula 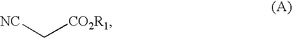
   wherein R₁ is alkyl or benzyl, to a mixture of a chiral cyclopentanone of formula 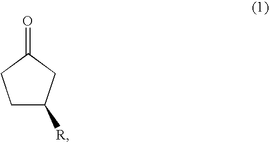
   a solvent, a carboxylic acid, and a Knoevenagel reaction catalyst, and stirring the mixture in the presence of a means of removing water to produce the alkene of formula 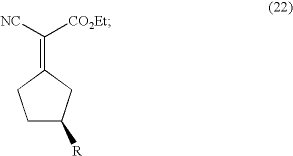
b) adding the product of Step a) above to a mixture of benzylmagnesium chloride or benzylmagnesium iodide, in a solvent to produce the addition of products of formulas 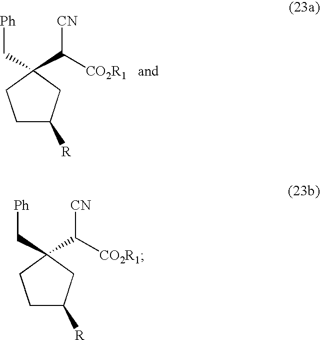
c) adding the products of Step b) above to a mixture of a base selected from potassium hydroxide, sodium hydroxide, lithium hydroxide, and cesium hydroxide, in a solvent, and stirring, and then acidifying to produce the carboxylic acids of formulas 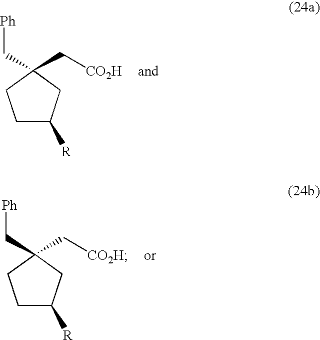
   adding the products of Step b) above to an acid mixture, and stirring to produce the carboxylic acids of formulas 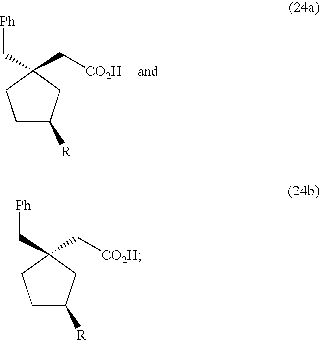
d) contacting the products of Step c) above with an amine in a solvent, and recrystallizing the salt so formed to produce the enriched diastereomer of formula 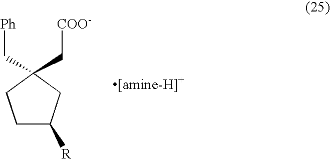
   as the amine salt: and
e) converting the product of Step d) to a carboxylic acid of formula 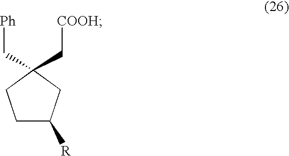
f) adding the product of Step e) to a mixture of iodomethane, a solvent, and 1,8-diazabicyclo[5.4.0]undec-7-ene (DBU), and stirring to produce the ester of formula 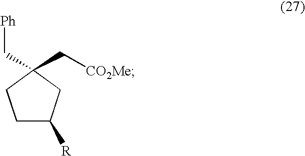
   or adding the product of Step e) to methanol and an acid to produce the ester of formula (27) 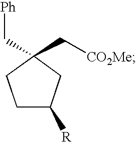
   or adding the product of Step e) to a solution of diazomethane or trimethylsilyl-diazomethane in a solvent to produce ester of formula 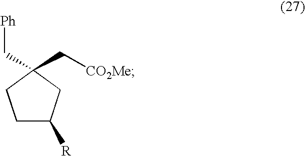
g) adding the product of Step f) to a mixture of carbon tetrachloride or ethyl acetate, and acetonitrile, water, sodium periodate, and ruthenium(II) chloride, and stirring to produce the carboxylic acid of formula 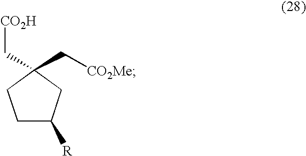
h) adding the product of Step g) to a mixture of a tertiary amine base, a solvent, and diphenylphosphoryl azide (DPPA), and stirring to produce the isocyanate of formula 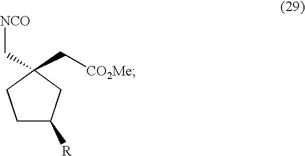
   or adding the product of Step g) above to ethyl chloroformate or isobutyl chloroformate and a base in a solvent at a temperature of from −40° C. to 78° C., followed by adding a solution of sodium azide in water and tetrahydrofuran or acetone, followed by adding toluene or benzene, and refluxing to produce isocyanate of formula 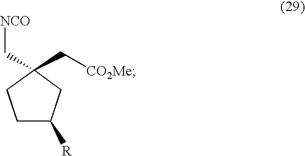
i) adding the product of Step h) to a mixture of a solvent and methanol, and stirring to produce the carbamate of formula 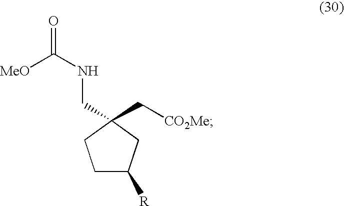
j) adding the product of Step i) to a mixture of a solvent and aqueous hydrochloric acid, and stirring to produce a compound of formula 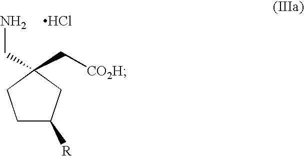
k) converting the product of Step j) to a compound of formula 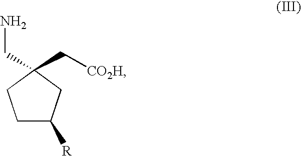
   and further converting, if desired, to a pharmaceutically acceptable salt by known means.

This process is outlined in Scheme 4.

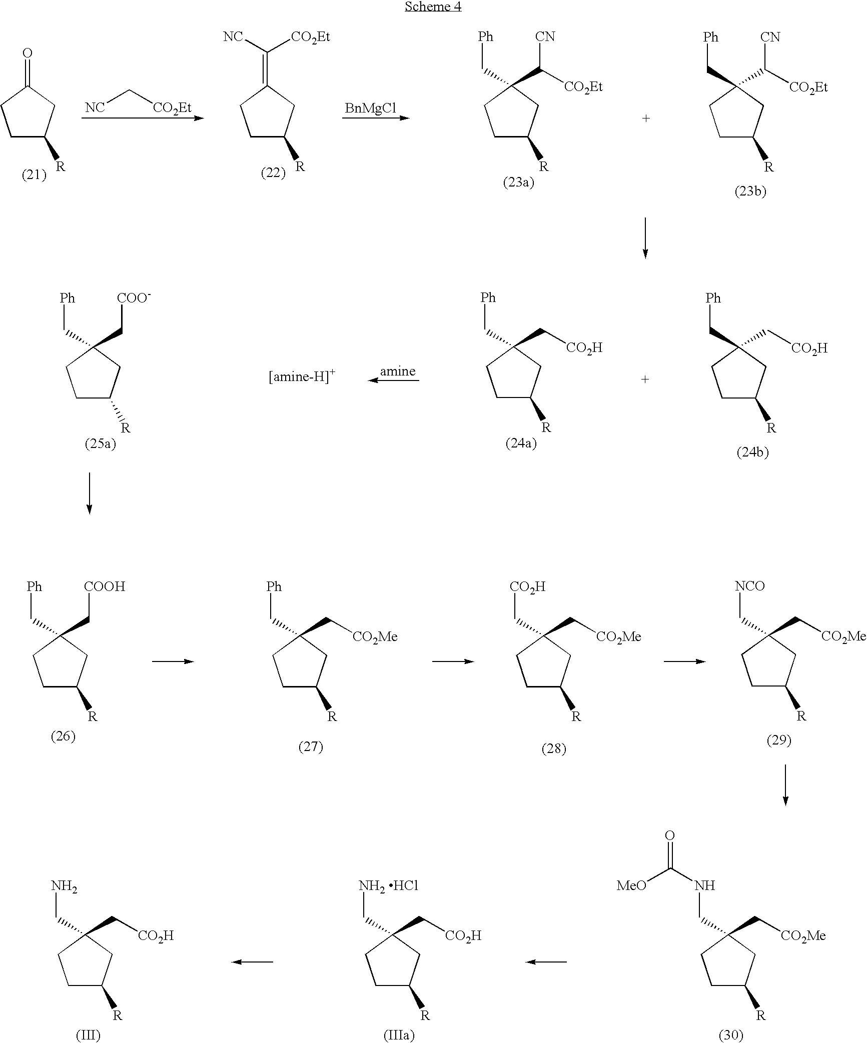

Preferred is a process for the preparation of a compound of Formula III wherein R is C₁-C₁₀ alkyl or C₃-C₁₀ cycloalkyl, and pharmaceutically acceptable salts thereof, which comprises:
a) adding ethyl cyanoacetate to a mixture of a chiral cyclopentanone of formula (21) in a solvent selected from toluene, benzene, xylenes, or n-heptane to which acetic acid and β-alanine or ammonium acetate were added, and stirring the mixture at a temperature from 0° C. to 150° C. to produce the alkene of formula (22).
b) adding the product of Step a) above to a mixture of benzylmagnesium chloride in a dry solvent selected from tetrahydrofuran, 1,4-dioxane, n-heptane, toluene, ethyl ether, or tert-butyl methyl ether at a temperature from −100° C. to 110° C. to produce the addition products of formulas (23a) and (23b);
c) adding the products of Step b) above to a mixture of a base selected from potassium hydroxide, sodium hydroxide, lithium hydroxide, or cesium hydroxide in a solvent selected from ethylene glycol, 2-methoxyethyl ether, 1,4-dioxane, or diethylene glycol and stirring the mixture at a temperature from 25° C. to 250° C. to produce the carboxylic acids of formulas (24a) and (24b);
d) contacting the products of Step c) above with (R)-α-methyl-benzylamine in a solvent selected from ethyl acetate, acetonitrile, tetrahydrofuran, or 1,4-dioxane at a temperature from −40° C. to 105° C., and recrystallizing the salt so formed from a solvent selected from ethyl acetate, acetonitrile, tetrahydrofuran, 1,4-dioxane, tert-butyl methyl ether, toluene, or n-heptane to produce the enriched diastereomer of formula (25a) as the (R)-α-methyl-benzylamine salt.
e) adding the product of Step d) to a mixture selected from aqueous hydrochloric acid, hydrochloric acid dissolved in acetic acid, or hydrochloric acid dissolved in acetic acid to which water was added and stirring at a temperature from −40° C. to 115° C. to produce the carboxylic acid of formula (26);
f) adding the product of Step e) to a mixture of iodomethane in a solvent selected from dichloromethane, chloroform, tetrahydrofuran, toluene, or 1,4-dioxane to which 1.8-diazabicyclo[5.4.0]undec-7-ene (DBU) was added, and stirring at a temperature from −40° C. to 110° C. to produce the ester of formula (27);
g) adding the product of Step f) to a mixture of carbon tetrachloride and acetonitrile to which water, sodium periodate, and ruthenium(III) chloride were added, and stirring at a temperature from −40° C. to 80° C. to produce the carboxylic acid of formula (28);
h) adding the product of Step g) to a mixture of a base selected from triethylamine or diisopropylethylamine and a solvent selected from toluene, benzene, xylenes, or n-heptane to which diphenylphosphoryl azide (DPPA) was added, and stirring at a temperature from 0° C. to 150° C. to produce the isocyanate of formula (29);
i) adding the product of Step h) to a solvent selected from toluene, benzene, xylenes, or n-heptane to which methanol was added and stirring at a temperature from 0° C. to 150° C. to produce the carbamate of formula (30);
j) adding the product of Step i) to a solvent selected from water, acetic acid, or 1,4-dioxane to which aqueous hydrochloric acid at a concentration of from 0.01 M to 12 M was added, and stirring at a temperature from 0° C. to 115° C. to produce a compound of Formula IIIa;
k) converting the product of Step j) to a compound of Formula III, and further converting, if desired, to a pharmaceutically acceptable salt by known means.

More preferred is a process for the preparation of a compound of Formula III wherein R is C₁-C₁₀ alkyl or C₃-C₁₀ cycloalkyl, and pharmaceutically acceptable salts thereof, which comprises:
a) adding ethyl cyanoacetate to a mixture of a chiral cyclopentanone of formula (21) in toluene to which acetic acid and ammonium acetate were added, and heating the mixture at reflux to produce the alkene of formula (22):
b) adding the product of Step a) above to a mixture of benzylmagnesium chloride in dry tetrahydrofuran at −100° C. to −20° C. to produce the addition products of formulas (23a) and (23b);
c) adding the products of Step b) above to a mixture of potassium hydroxide in ethylene glycol, and heating the mixture at 100° C. to 200° C. to produce the hydrolysis products of formulas (24a) and (24b);
d) contacting the products of Step c) above with (R)-α-methyl-benzylamine in ethyl acetate, and recrystallizing the salt so formed from ethyl acetate to produce the enriched diastereomer of formula (25a) as the (R)-α-methyl-benzylamine salt;
e) adding the product of Step d) to aqueous hydrochloric acid and stirring to produce the carboxylic acid of formula (26);
f) adding the product of Step e) to a mixture of iodomethane in dichloromethane to which 1,8-diazabicyclo[5.4.0]undec-7-ene (DBU) was added, and stirring to produce the ester of formula (27);
g) adding the product of Step f) to a mixture of carbon tetrachloride and acetonitrile to which water, sodium periodate, and ruthenium(II) chloride were added, and stirring to produce the carboxylic acid of formula (28);
h) adding the product of Step g) to a mixture of triethylamine and toluene to which diphenylphosphoryl azide (DPPA) was added, and refluxing to produce the isocyanate of formula (29);
i) adding the product of Step h) to a mixture of methanol and toluene, and refluxing to produce the carbamate of formula (30);
j) adding the product of Step i) to 1,4-dioxane to which aqueous hydrochloric acid at a concentration of 6 M was added, and stirring to produce a compound of Formula IIIa;
k) converting the product of Step j) to a compound of Formula III, and further convening if desired, to a pharmaceutically acceptable salt by known means.

Also preferred is a process for the preparation of a compound of Formula III, further characterized in that the intermediate product 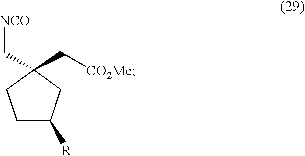
formed is further reacted, without isolation, with methanol to produce the carbamate of formula

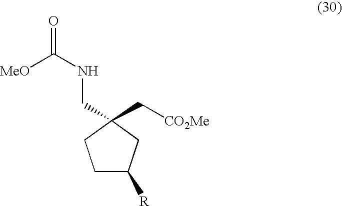

Further, the invention provides a process for the preparation of a compound of Formula IV 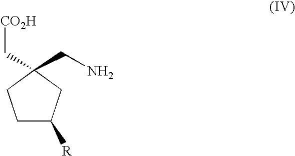
wherein R is C₁-C₁₀ alkyl or C₃-C₁₀ cycloalkyl, and pharmaceutically acceptable salts thereof, which comprises:
a) adding a cyanoacetate of formula 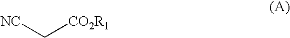
   wherein R₁ is alkyl or benzyl, to a mixture of a chiral cyclopentanone of formula 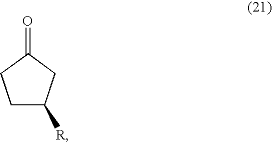
   a solvent, a carboxylic acid, and a Knoevenagel reaction catalyst, and stirring the mixture in the presence of a means of removing water to produce the alkene of formula 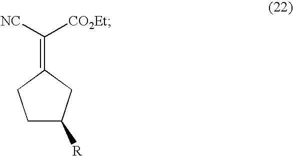
b) adding the product of Step a) above to a mixture of benzylmagnesium chloride or benzylmagnesium iodide, in a solvent to produce the addition of products of formulas 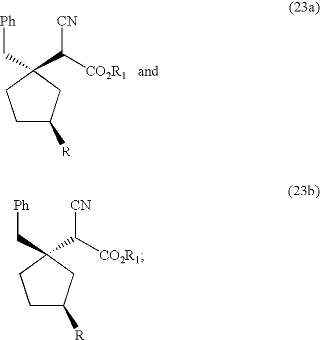
c) adding the products of Step b) above to a mixture of a base selected from potassium hydroxide, sodium hydroxide, lithium hydroxide, and cesium hydroxide, in a solvent, and stirring, and then acidifying to produce the carboxylic acids of formulas 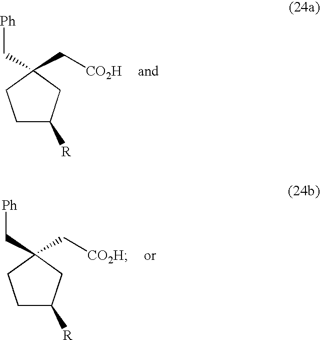
adding the products of Step b) above to an acid mixture, and stirring to produce the carboxylic acids of formulas 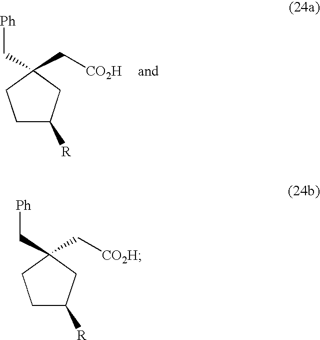
d) contacting the products of Step c) above with an amine in a solvent, and recrystallizing the salt so formed to produce the enriched diastereomer of formula 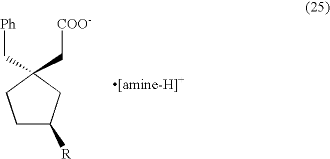
   as the amine salt; and
e) converting the product of Step d) to a carboxylic acid of formula 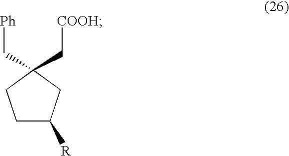
f) adding the product of Step e) to a mixture of a tertiary amine base, a solvent, and diphenylphosphoryl azide (DPPA) is added, and stirring to produce the isocyanate of formula 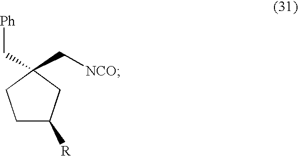
   or adding the product of Step g) above to ethyl chloroformate or isobutyl chloroformate and a base in a solvent at a temperature of from −40° C. to 78° C. followed by adding a solution of sodium azide in water and tetrahydrofuran or acetone, followed by adding toluene or benzene, and refluxing to produce the isocyanate of formula 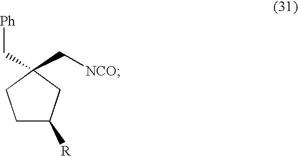
g) adding the product of Step f) to a mixture of a solvent and methanol, and stirring to produce the carbamate of formula 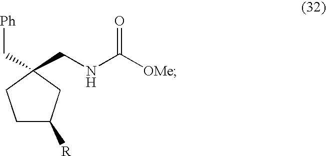
h) adding the product of Step g) to a mixture of carbon tetrachloride or ethyl acetate, and acetonitrile, water, sodium periodate, and ruthenium(III) chloride, and stirring to produce the carboxylic acid of formula 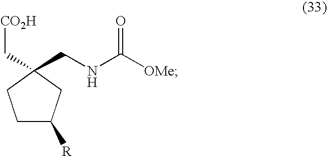
i) adding the product of Step h) to a mixture of a solvent and aqueous hydrochloric acid, and stirring to produce a compound of formula 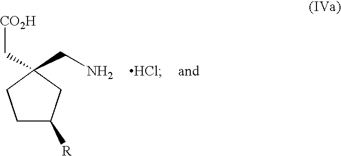
j) converting the product of Step i) to a compound of formula 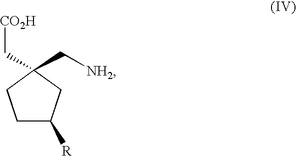
   and further converting, if desired, to a pharmaceutically acceptable salt by known means.

This process is outlined in Scheme 5.

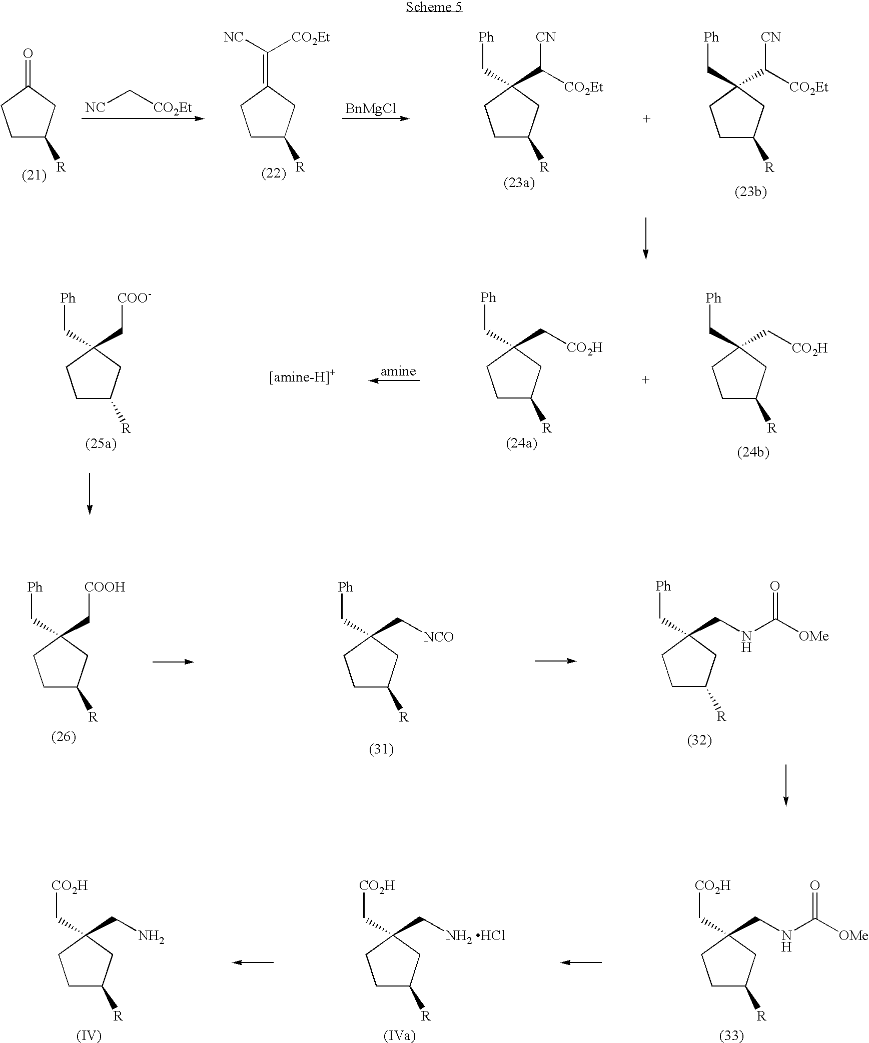

Preferred is a process for the preparation of a compound of Formula IV wherein R is C₁-C₁₀ alkyl or C₃-C₁₀ cycloalkyl, and pharmaceutically acceptable salts thereof, which comprises:
a) adding ethyl cyanoacetate to a mixture of a chiral cyclopentanone of formula (21) in a solvent selected from toluene, benzene, xylenes, or n-heptane to which acetic acid and β-alanine or ammonium acetate were added, and stirring the mixture at a temperature from 0° C. to 150° C. to produce the alkene of formula (22);
b) adding the product of Step a) above to a mixture of benzylmagnesium chloride in a dry, solvent selected from tetrahydrofuran, 1,4-dioxane, n-heptane, toluene, ethyl ether, or tert-butyl methyl ether at a temperature from −100° C. to 110° C. to produce the addition products of formulas (23a) and (23b);
c) adding the products of Step b) above to a mixture of a base selected from potassium hydroxide, sodium hydroxide, lithium hydroxide, or cesium hydroxide in a solvent selected from ethylene glycol, 2-methoxyethyl ether, 1,4-dioxane, or diethylene glycol and stirring the mixture at a temperature from 25° C. to 250° C. to produce the carboxylic acids of formulas (24a) and (24b);
d) contacting the products of Step c) above with (R)-α-methyl-benzylamine in a solvent selected from ethyl acetate, acetonitrile, tetrahydrofuran, or 1,4-dioxane at a temperature from −40° C. to 105° C. and recrystallizing the salt so formed from a solvent selected from ethyl acetate, acetonitrile, tetrahydrofuran, 1,4-dioxane, tert-butyl methyl ether, toluene, or n-heptane to produce the enriched diastereomer of formula (25a) as the (R)-α-methyl-benzylamine salt;
e) adding the product of Step d) to a mixture selected from aqueous hydrochloric acid, hydrochloric acid dissolved in acetic acid, or hydrochloric acid dissolved in acetic acid to which water was added and stirring at a temperature from −40° C. to 115° C. to produce the carboxylic acid of formula (26):
f) adding the product of Step e) to a mixture of a base selected from triethylamine or diisopropylethylamine and a solvent selected from toluene, benzene, xylenes, or n-heptane to which diphenylphosphoryl azide (DPPA) was added, and stirring at a temperature from 0° C. to 150° C. to produce the isocyanate of formula (31);
g) adding the product of Step f) to a solvent selected from toluene, benzene, xylenes, or n-heptane to which methanol was added and stirring at a temperature from 0° C. to 150° C. to produce the carbamate of formula (32);
h) adding the product of Step g) to a mixture of carbon tetrachloride and acetonitrile to which water, sodium periodate, and ruthenium(III) chloride were added, and stirring at a temperature from −40° C. to 80° C. to produce the carboxylic acid of formula (33);
i) adding the product of Step h) to a solvent selected from water, acetic acid, or 1,4-dioxane to which aqueous hydrochloric acid at a concentration of from 0.01 M to 12 M was added, and stirring at a temperature from 0° C. to 115° C. to produce a compound of Formula IVa:
j) convening the product of Step i) to a compound of Formula IV, and further converting, if desired, to a pharmaceutically acceptable salt by known means.

More preferred is a process for the preparation of a compound of Formula IV wherein R is C₁-C₁₀ alkyl or C₃-C₁₀ cycloalkyl, and pharmaceutically acceptable salts thereof, which comprises
a) adding ethyl cyanoacetate to a mixture of a chiral cyclopentanone of formula (21) in toluene to which acetic acid and ammonium acetate were added, and heating the mixture at reflux to produce the alkene of formula (22);
b) adding the product of Step a) above to a mixture of benzylmagnesium chloride in dry tetrahydrofuran at −100° C. to −20° C. to produce the addition products of formulas (23a) and (23b);
c) adding the products of Step b) above to a mixture of potassium hydroxide in ethylene glycol, and heating the mixture at 100° C. to 200° C. to produce the hydrolysis products of formulas (24a) and (24b);
d) contacting the products of Step c) above with (R)-α-methyl-benzylamine in ethyl acetate, and recrystallizing the salt so formed from ethyl acetate to produce the enriched diastereomer of formula (25a) as the (R)-α-methyl-benzylamine salt;
e) adding the product of Step d) to aqueous hydrochloric acid and stirring to produce the carboxylic acid of formula (26);
f) adding the product of Step e) to a mixture of triethylamine and toluene to which diphenylphosphoryl azide (DPPA) was added, and refluxing to produce the isocyanate of formula (31);
g) adding the product of Step f) to a mixture of methanol and toluene, and refluxing to produce the carbamate of formula (32);
h) adding the product of Step g) to a mixture of carbon tetrachloride and acetonitrile to which water, sodium periodate, and ruthenium(III) chloride were added, and stirring to produce the carboxylic acid of formula (33);
i) adding the product of Step h) to 1,4-dioxane to which aqueous hydrochloric acid at a concentration of 6 M was added, and stirring to produce a compound of Formula IVa;
j) converting the product of Step i) to a compound of Formula IV, and further converting, if dried, to a pharmaceutically acceptable salt by known means.

Also preferably a process for the preparation of a compound of Formula IV, further characterized in that the intermediate product (31) 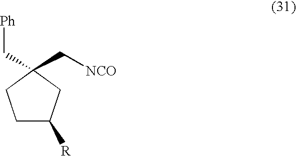
is further reacted, without isolation, with methanol to produce the carbamate of formula

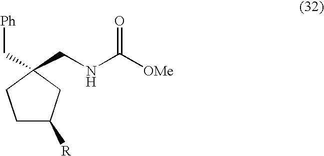

Still further, the invention provides a process for the preparation of a compound of Formula IV 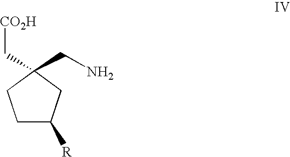
wherein R is C₁-C₁₀ alkyl or C₃-C₁₀ cycloalkyl, and pharmaceutically acceptable salts thereof, which comprises:
a) adding a cyanoacetate of formula 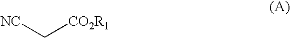
   wherein R₁ is alkyl or benzyl, to a mixture of a chiral cyclopentanone of formula 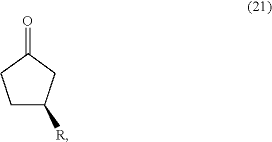
   a solvent, a carboxylic acid, and a Knoevenagel reaction catalyst, and stirring the mixture in the presence of a means of removing water to produce the alkene of formula 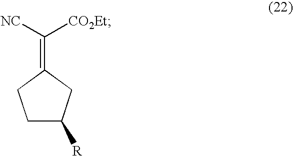
b) adding the product of Step a) above to a mixture of benzylmagnesium chloride or benzylmagnesium iodide, in a solvent to produce the addition of products of formulas 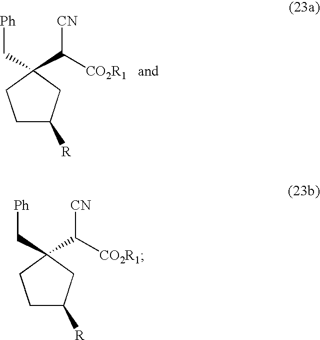
c) adding the products of Step b) above to a mixture of a base selected from potassium hydroxide, sodium hydroxide, lithium hydroxide, and cesium hydroxide, in a solvent, and stirring, and then acidifying to produce the carboxylic acids of formulas 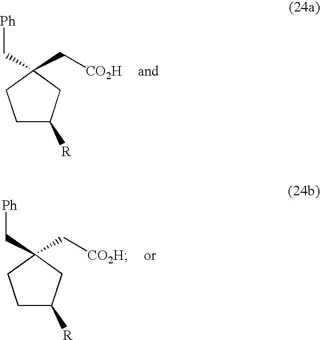
   adding the products of Step b) above to an acid mixture, and stirring to produce the carboxylic acids of formulas 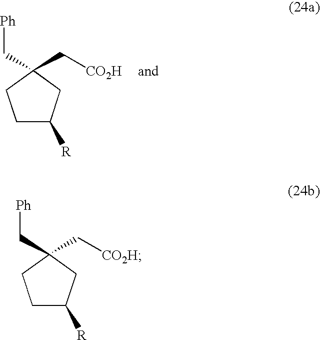
d) contacting the products of Step c) above with an amine in a solvent, and recrystallizing the salt so formed to produce the enriched diastereomer of formula 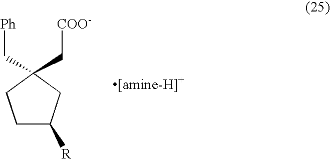
   as the amine salt; and
e) converting the product of Step d) to a carboxylic acid of formula 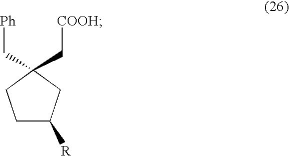
f) adding oxalyl chloride to a mixture of the product of Step e), a solvent, and N,N-dimethylformamide (DMF), and stirring to produce the acid chloride of formula 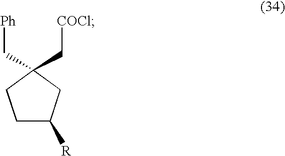
g) adding the product of Step f) to a mixture of tert-butyl alcohol, a solvent, and a tertiary amine base, and stirring to produce the ester of formula 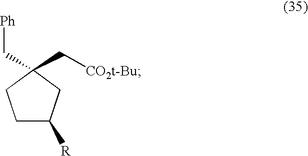
h) adding the product of Step g) to a mixture of carbon tetrachloride or ethyl acetate, and acetonitrile, water, sodium periodate, and ruthenium(III) chloride, and stirring to produce the carboxylic acid of formula 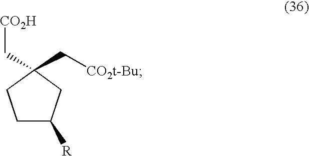
i) adding the product of Step h) to a mixture of a solvent, methanol, and (trimethylsilyl)diazomethane, and stirring to produce the bis ester of formula 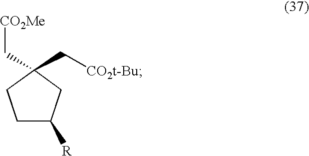
   or adding the product of Step h) to a mixture of iodomethane, a solvent, and a base, and stirring to produce the bis ester of formula 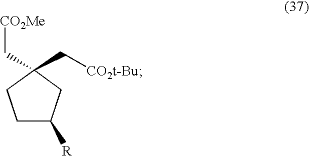
j) adding an acid to a mixture of the product from Step i) and a solvent and stirring to produce the carboxylic acid of formula 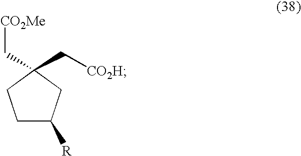
k) adding the product of Step j) to a mixture of a tertiary amine base, a solvent, and diphenylphosphoryl azide (DPPA), and stirring to produce the isocyanate of formula 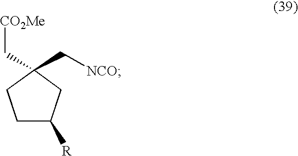
   or adding the product of Step j) above to ethyl chloroformate or isobutyl chloroformate and a base in a solvent at a temperature of from −40° C. to 78° C. followed by adding a solution of sodium azide in water and tetrahydrofuran or acetone, followed by adding toluene or benzene, and refluxing to produce isocyanate of formula 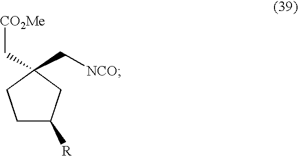
l) adding the product of Step k) to a mixture of a solvent and methanol, and stirring to produce the carbamate of formula 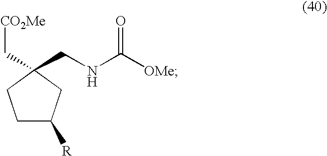
m) adding the product of Step l) to a mixture of a solvent and hydrochloric acid, and stirring to produce a compound of formula 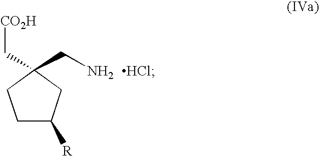
n) converting the product of Step m) to a compound of Formula IV 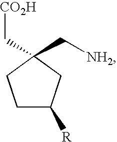
   and further converting, if desired, to a pharmaceutically acceptable salt by known means.

This process is outlined in Scheme 6.

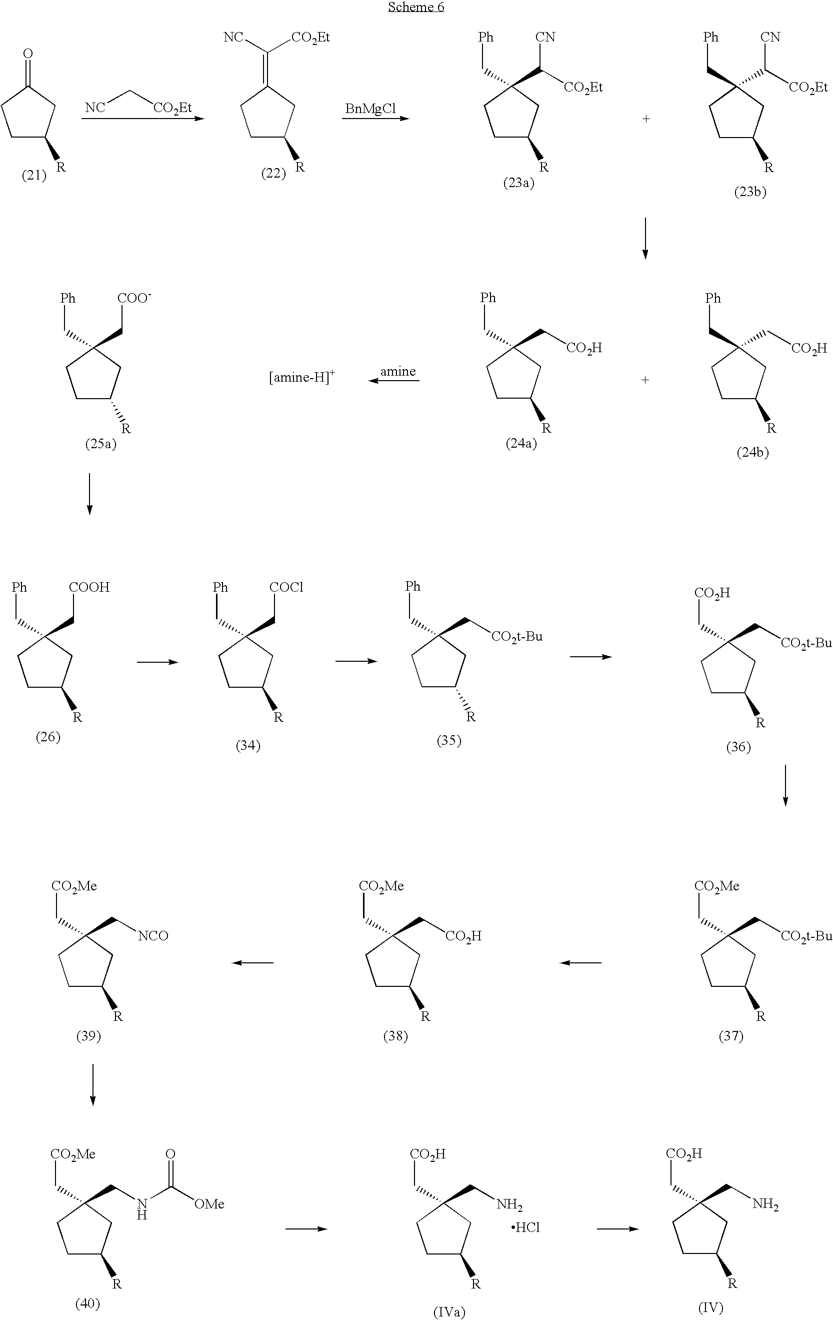

Preferred is a process for the preparation of a compound of Formula IV wherein R is C₁-C₁₀ alkyl or C₃-C₁₀ cycloalkyl, and pharmaceutically acceptable salts thereof, which comprises:
a) adding ethyl cyanoacetate to a mixture of a chiral cyclopentanone of formula (21) in a solvent selected from toluene, benzene, xylenes, or n-heptane to which acetic acid and β-alanine or ammonium acetate were added, and stirring the mixture at a temperature from 0° C. to 150° C. to produce the alkene of formula (22);
b) adding the product of Step a) above to a mixture of benzylmagnesium chloride in a dry solvent selected from tetrahydrofuran, 1,4-dioxane, n-heptane, toluene, ethyl ether, or tert-butyl methyl ether at a temperature from −100° C. to 110° C. to produce the addition products of formulas (23a) and (23b);
c) adding the products of Step b) above to a mixture of a base selected from potassium hydroxide, sodium hydroxide, lithium hydroxide, or cesium hydroxide in a solvent selected from ethylene glycol, 2-methoxyethyl ether, 1,4-dioxane, or diethylene glycol and stirring the mixture at a temperature from 25° C. to 250° C. to produce the carboxylic acids of formulas (24a) and (24b);
d) contacting the products of Step c) above with (R)-α-methyl-benzylamine in a solvent selected from ethyl acetate, acetonitrile, tetrahydrofuran, or 1,4-dioxane at a temperature from −40° C. to 105° C., and recrystallizing the salt so formed from a solvent selected from ethyl acetate, acetonitrile, tetrahydrofuran, 1,4-dioxane, tert-butyl methyl ether, toluene, or n-heptane to produce the enriched diastereomer of formula (25a) as the (R)-α-methyl-benzylamine salt,
e) adding the product of Step d) to a mixture selected from aqueous hydrochloric acid, hydrochloric acid dissolved in acetic acid, or hydrochloric acid dissolved in acetic acid to which water was added and stirring at a temperature from −40° C. to 115° C. to produce the carboxylic acid of formula (26);
f) adding oxalyl chloride to a mixture of the product of Step e) and a solvent selected from dichloromethane, chloroform, ethyl, ether, toluene, or tert-butyl methyl ether to which 0.01 mol percent to 10 mol percent of N,N-dimethylformamide (DMF) was added, and stirring at a temperature from −40° C. to 110° C. to produce the acid chloride of formula (34);
g) adding the product of Step f) to a mixture of tert-butyl alcohol in a solvent selected from dichloromethane, chloroform, ethyl ether, toluene, or tert-butyl methyl ether to which N,N-diisopropylethylamine (DIPEA) or triethylamine was added, and stirring at a temperature from −40° C. to 110° C. to produce the ester of formula (35);
h) adding the product of Step g) to a mixture of carbon tetrachloride and acetonitrile to which water, sodium periodate, and ruthenium(III) chloride were added, and stirring at a temperature from −40° C. to 80° C. to produce the carboxylic acid of formula (36);
i) adding the product of Step h) to a solvent selected from toluene, benzene, xylenes, or n-heptane to which methanol and (trimethylsilyl)diazomethane were added, and stirring at a temperature from 0° C. to 150° C. to produce the bis ester of formula (37);
j) adding trifluoroacetic acid (TFA) to a mixture of the product from Step i) and a solvent selected from dichloromethane, chloroform, 1,4-dioxane, tetrahydrofuran, ethyl ether, or tert-butyl methyl ether and stirring at a temperature from −40° C. to 110° C. to produce the carboxylic acid of formula (38);
k) adding the product of Step j) to a mixture of a base selected from triethylamine or diisopropylethylamine and a solvent selected from toluene, benzene, xylenes, or n-heptane to which diphenylphosphoryl azide (DPPA) was added, and stirring at a temperature from 0° C. to 150° C. to produce the isocyanate of formula (39);
l) adding the product of Step k) to a solvent selected from toluene, benzene, xylenes, or n-heptane to which methanol was added and stirring at a temperature from 0° C. to 150° C. to produce the carbamate of formula (40);
m) adding the product of Step l) to a solvent selected from water, acetic acid, or 1,4-dioxane to which aqueous hydrochloric acid at a concentration of from 0.01 M to 12 M was added, and stirring at a temperature from 0° C. to 115° C. to produce a compound of Formula IVa;
n) converting the product of Step m) to a compound of Formula IV, and further converting, if desired, to a pharmaceutically acceptable salt by known means.

More preferred is a process for the preparation of a compound of Formula IV wherein R is C₁-C₁₀ alkyl or C₃-C₁₀ cycloalkyl, and pharmaceutically acceptable salts thereof, which comprises:
a) adding ethyl cyanoacetate to a mixture of a chiral cyclopentanone of formula (21) in toluene to which acetic acid and ammonium acetate were added, and heating the mixture at reflux to produce the alkene of formula (22);
b) adding the product of Step a) above to a mixture of benzylmagnesium chloride in dry tetrahydrofuran at −100° C. to −20° C. to produce the addition products of formulas (23a) and (23b);
c) adding the products of Step b) above to a mixture of potassium hydroxide in ethylene glycol, and heating the mixture at 100° C. to 200° C. to produce the hydrolysis products of formulas (24a) and (24b);
d) contacting the products of Step c) above with (R)-α-methyl-benzylamine in ethyl acetate, and recrystallizing the salt so formed from ethyl acetate to produce the enriched diastereomer of formula (25a) as the (R)-α-methyl-benzylamine salt;
e) adding the product of Step d) to aqueous hydrochloric acid and stirring to produce the carboxylic acid of formula (26);
f) adding oxalyl chloride to a mixture of the product of Step e) and dichloromethane to which a catalytic amount of N,N-dimethylformamide (DMF) was added, and stirring to produce the acid chloride of formula (34);
g) adding the product of Step f) to a mixture of tert-butyl alcohol in dichloromethane to which N,N-diisopropylethylamine (DIPEA) was added, and stirring to produce the ester of formula (35);
h) adding the product of Step g) to a mixture of carbon tetrachloride and acetonitrile to which water, sodium periodate, and ruthenium(III) chloride were added, and stirring to produce the carboxylic acid of formula (36);
i) adding the product of Step h) to a mixture of methanol and toluene to which (trimethylsilyl)diazomethane was added, and stirring to produce the bis ester of formula (37);
j) adding trifluoroacetic acid (TFA) to a mixture of the product from Step i) and dichloromethane, and stirring to produce the carboxylic acid of formula (38);
k) adding the product of Step j) to a mixture of triethylamine and toluene to which diphenylphosphoryl azide (DPPA) was added, and refluxing to produce the isocyanate of formula (39);
l) adding the product of Step k) to a mixture of methanol and toluene, and refluxing to produce the carbamate of formula (40);
m) adding the product of Step l) to 1,4-dioxane to which aqueous hydrochloric acid at a concentration of 6 M was added, and stirring to produce a compound of Formula IVa;
n) converting the product of Step m) to a compound of Formula IV, and further convening, if desired, to a pharmaceutically acceptable salt by known means.

Also preferred is a process for the preparation of a compound of Formula IV, further characterized in that the intermediate product 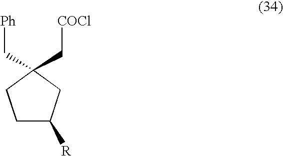
formed is further reacted, without isolation, with tert-butyl alcohol to produce the ester of formula

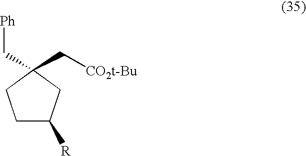

Also preferred is a process for the preparation of a compound of Formula IV, further characterized in that the intermediate product 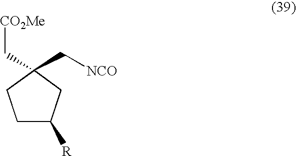
formed is further reacted, without isolation, with methanol to produce the carbamate of formula

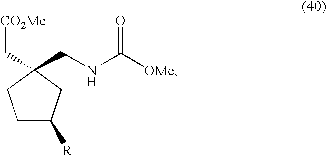

Also preferred is a process for the preparation of a compound of Formula IV, further characterized in that the intermediate product 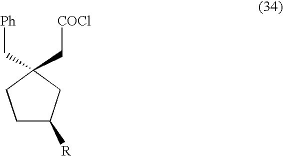
formed is further reacted, without isolation, with tert-butyl alcohol to produce the ester of formula 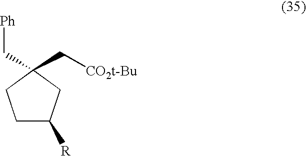
and the intermediate product 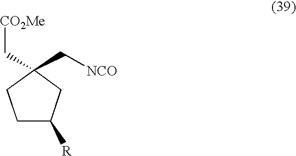
formed is further reacted, without isolation, with methanol to produce the carbamate of formula

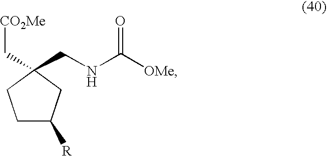

Further, the invention provides a process for the preparation of a compound of formula (6) 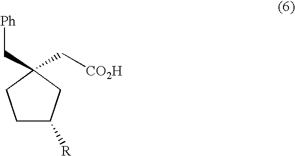
wherein R is C₁-C₁₀ alkyl or C₃-C₁₀ cycloalkyl, and pharmaceutically acceptable salts thereof, which comprises:
a) adding a cyanoacetate of formula 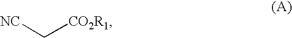
   wherein R₁ is alkyl or benzyl, to a mixture of a chiral cyclopentanone of formula 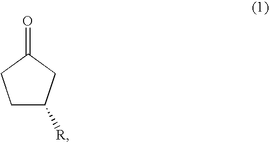
   a solvent, a carboxylic acid, and a Knoevenagel reaction catalyst, and stirring the mixture in the presence of a means of removing water to produce the alkene of formula 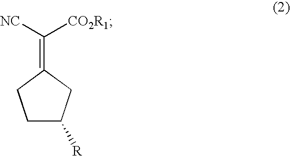
b) adding the product of Step a) above to a mixture of benzylmagnesium chloride, benzylmagnesium bromide, or benzylmagnesium iodide, in a solvent to produce the addition products of formulas 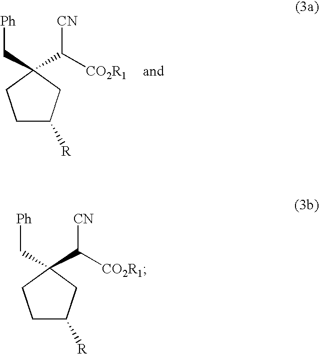
c) adding the products of Step b) above to a mixture of a base selected from potassium hydroxide, sodium hydroxide, lithium hydroxide, and cesium hydroxide, and a solvent, and stirring, and then acidifying to produce the carboxylic acids of formulas 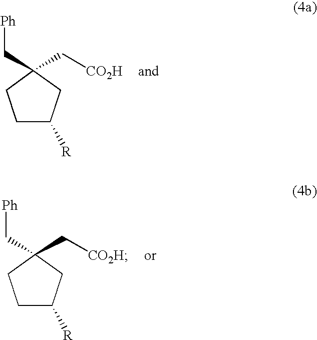
   adding the products of Step b) above to an acid mixture and stirring to produce the carboxylic acids of formulas 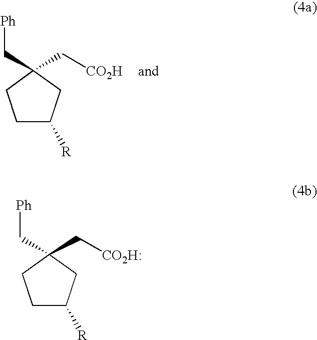
d) contacting the products of Step c) above with an amine in a solvent, and recrystallizing the salt so formed to produce the enriched diastereomer of formula 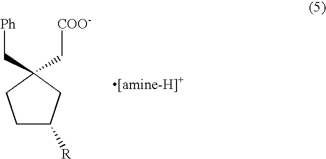
   as the amine salt; and
e) converting the product of Step d) to a carboxylic acid of formula

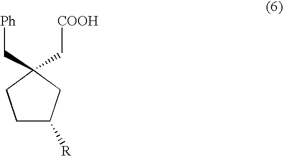

This process is outlined in Scheme 7.

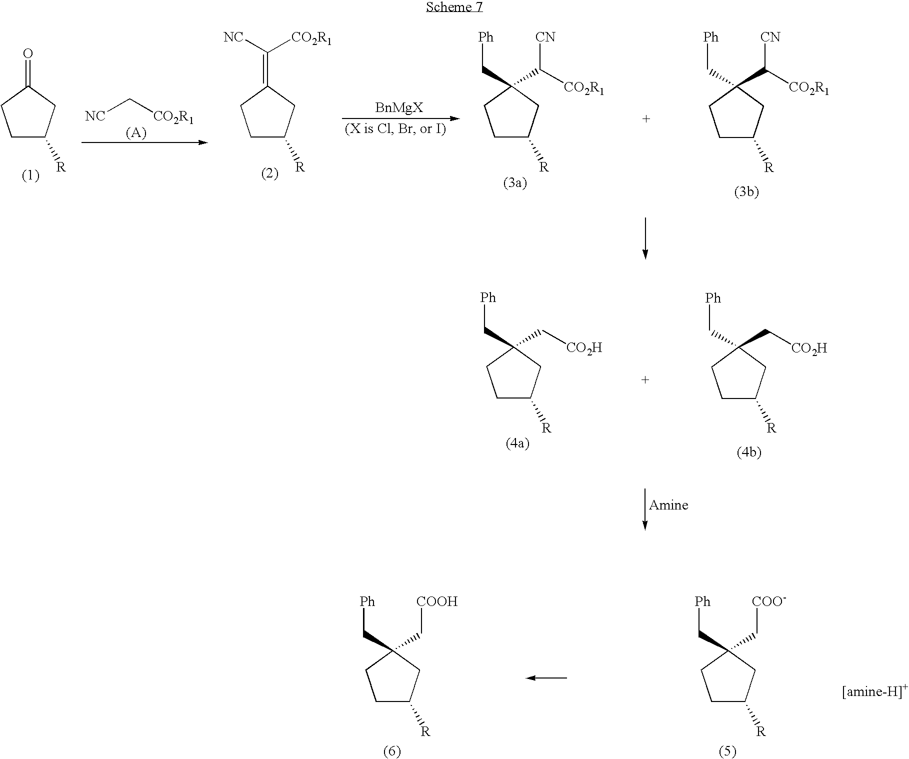

Preferred is a process for the preparation of a compound of formula (6) wherein R is C₁-C₁₀ alkyl or C₃-C₁₀ cycloalkyl, and pharmaceutically acceptable salts thereof, which comprises:
a) adding a cyanoacetate of formula 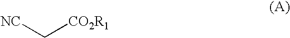
   wherein R₁ is selected from methyl, ethyl, n-propyl, iso-propyl, n-butyl, iso-butyl, sec-butyl, tert-butyl, and benzyl to a mixture of a chiral cyclopentanone of formula 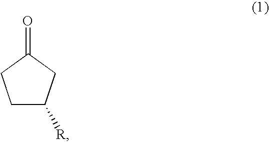
   a solvent selected from tetrahydrofuran, 1,4-dioxane, tert-butylmethylether, chloroform, dichloromethane, acetonitrile, ethyl ether, ethyl acetate, hexanes, N,N-dimethylformamide, dimethylsulfoxide, ethanol, tert-butanol, toluene, benzene, xylenes, and n-heptane, acetic acid, and a Knoevenagel reaction catalyst selected from β-alanine, ammonium acetate, and piperidine, and stirring the mixture in the presence of a means of removing water selected from azeotropic distillation, activated molecular sieves, anhydrous magnesium sulfate, anhydrous sodium sulfate, anhydrous sodium carbonate, anhydrous potassium carbonate, anhydrous cesium carbonate, trimethyl orthoformate, and triethyl orthoformate to produce the alkene of formula 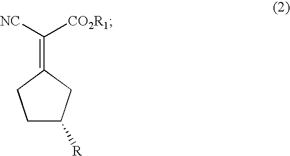
b) adding the product of Step a) above to a mixture of benzylmagnesium chloride, benzylmagnesium bromide, or benzylmagnesium iodide in a solvent selected from tetrahydrofuran, benzene, 1,4-dioxane, hexanes, n-heptane, toluene, diethyl ether, and tert-butyl methyl ether to produce the addition products of formulas 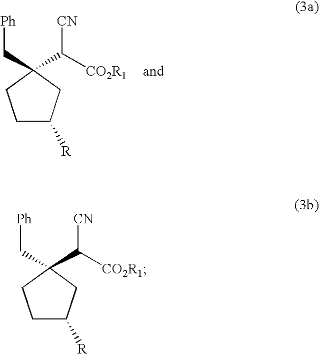
c) adding the products of Step b) above to a mixture of a base selected from potassium hydroxide, sodium hydroxide, lithium hydroxide, and cesium hydroxide in a solvent selected from ethylene glycol, 2-methoxyethyl ether, 1,4-dioxane, and diethylene glycol, and stirring the mixture, and then acidifying to produce the carboxylic acids of formulas 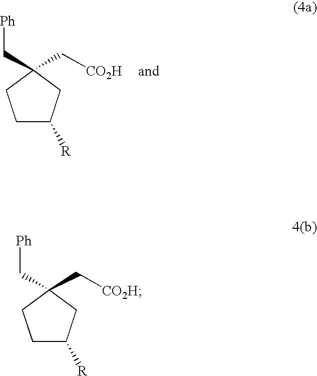
   or adding the products of Step b) above to an acid mixture selected from 6-12 M HCl, 12 M H₂SO₄, 10%-48% wt/wt hydrobromic acid, and HBr in aqueous acetic acid, and stirring to produce the carboxylic acids of formulas 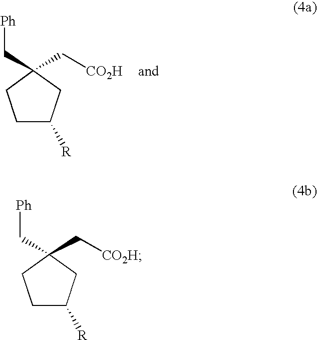
d) contacting the products of Step c) above with an amine selected from (S)-α-methyl-benzylamine, (R)-α-methyl-benzylamine, (R)-(+)-1-(naphthyl)ethylamine, (S)-(+)-1-(naphthyl)ethylamine, triethylamine, diisopropylethylamine, dicyclohexylamine, benzylamine, dibenzylamine, morpholine, N-methylmorpholine, piperidine, N-methylpiperidine, and pyridine in a solvent selected from N,N-dimethylformamide, chloroform, benzene, xylenes, hexanes, acetone, ethanol, methanol, iso-propanol, diethyl ether, dichloromethane, benzene, toluene, n-pentane, n-hexane, n-heptane, ethyl acetate, acetonitrile, tert-butyl methyl ether, tetrahydrofuran, and 1,4-dioxane, and recrystallizing the salt so formed to produce the enriched diastereomer of formula 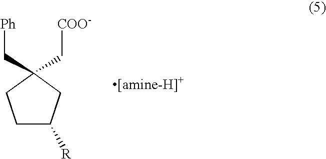
   as the amine salt; and
e) adding the product of Step d) to a mixture selected from aqueous hydrochloric acid, aqueous sulfuric acid, aqueous acetic acid, hydrochloric acid dissolved in acetic acid, and hydrochloric acid dissolved in acetic acid and water, and stirring to produce the carboxylic acid of formula 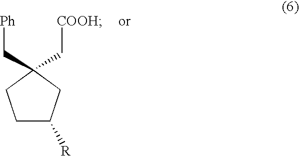
   partitioning the product of Step d) between a mixture of aqueous hydrochloric acid and a solvent selected from chloroform, dichloromethane, ethyl acetate, ethyl ether, tetrahydrofuran, 1,4-dioxane, toluene, and tert-butylmethylether, and drying and evaporating the organic layer to produce the carboxylic acid of formula

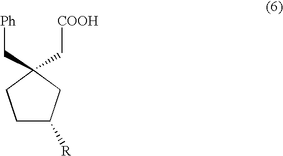

More preferred is a process for the preparation of a compound of formula (6) wherein R is C₁-C₁₀ alkyl or C₃-C₁₀ cycloalkyl, and pharmaceutically acceptable salts thereof, which comprises
a) adding a cyanoacetate of formula 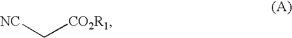
   wherein R₁ is ethyl, to a mixture of a chiral cyclopentanone of formula 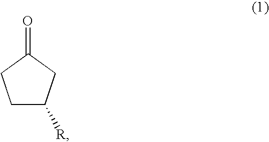
   toluene, acetic acid, and a Knoevenagel reaction catalyst which is ammonium acetate, and heating the mixture at reflux over a Dean-Stark trap to produce the alkene of formula 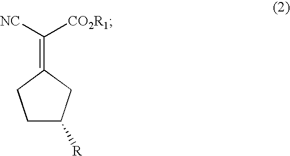
b) adding the product of Step a) above to a mixture of benzylmagnesium chloride in dry tetrahydrofuran at −100° C. to 25° C. to produce the addition products of formulas 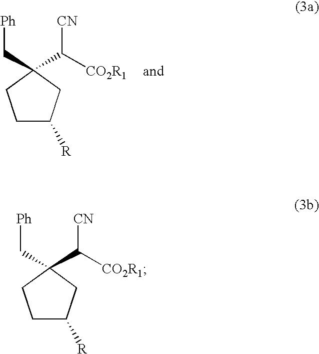
c) adding the products of Step b) above to a mixture of potassium hydroxide in ethylene glycol, and heating the mixture at 100° C. to 200° C., and then acidifying to produce the hydrolysis products of formulas 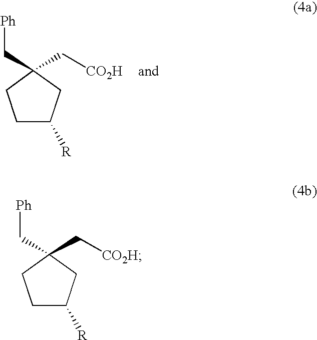
d) contacting the products of Step c) above with (S)-α-methyl-benzylamine in ethyl acetate, and recrystallizing the salt so formed from ethyl acetate to produce the enriched diastereomer of formula 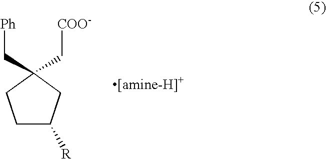
   as the (S)-α-methyl-benzylamine salt
e) adding the product of Step d) to aqueous hydrochloric acid and stirring to produce the carboxylic acid of formula

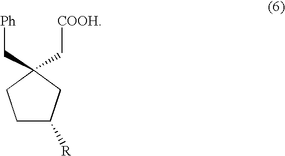

Further, the invention provides a process for the preparation of a compound of formula 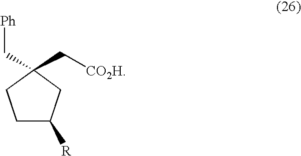
wherein R is C₁-C₁₀ alkyl or C₃-C₁₀ cycloalkyl, and pharmaceutically acceptable salts thereof, which comprises
a) adding a cyanoacetate of formula 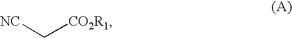
   wherein R₁ is alkyl or benzyl, to a mixture of a chiral cyclopentanone of formula 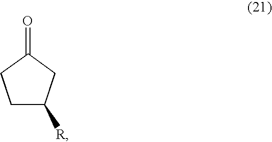
   a solvent, a carboxylic acid, and a Knoevenagel reaction catalyst, and stirring the mixture in the presence of a means of removing water to produce the alkene of formula 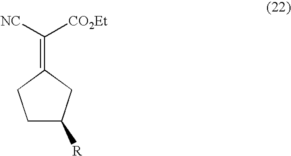
b) adding the product of Step a) above to a mixture of benzylmagnesium chloride or benzylmagnesium iodide, in a solvent to produce the addition of products of formulas 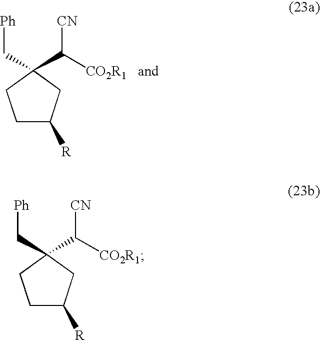
c) adding the products of Step b) above to a mixture of a base selected from potassium hydroxide, sodium hydroxide, lithium hydroxide, and cesium hydroxide, and a solvent, and stirring, and then acidifying to produce the carboxylic acids of formulas 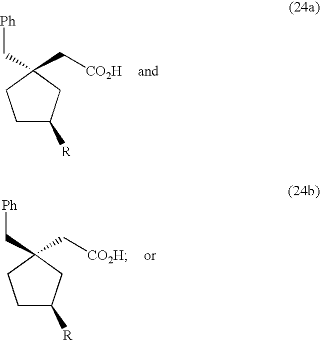
   adding the products of Step b) above to an acid mixture, and stirring to produce the carboxylic acids of formulas 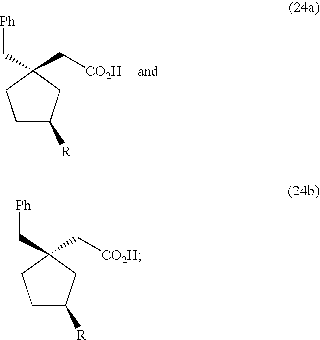
d) contacting the products of Step c) above with an amine in a solvent, and recrystallizing the salt so formed to produce the enriched diastereomer of formula 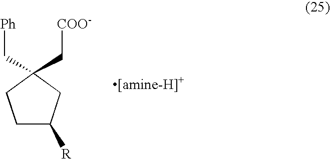
   as the amine salt; and
e) converting the product of Step d) to a carboxylic acid of formula

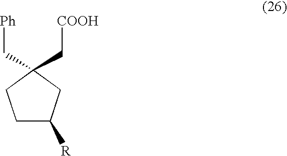

This process is outlined in Scheme 8.

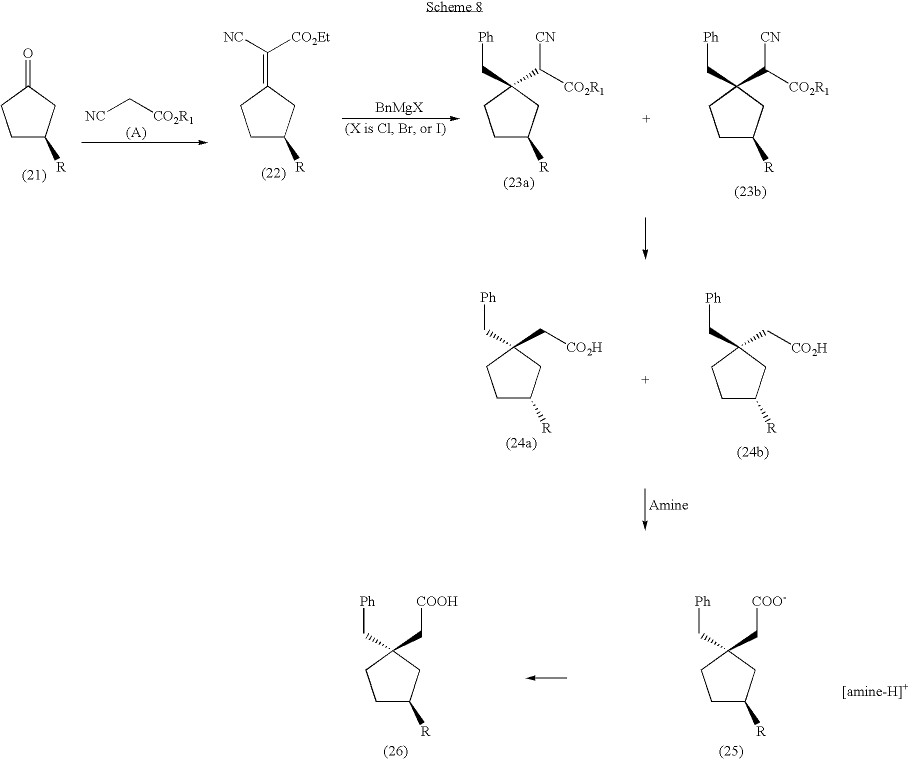

Preferred is a process for the preparation of a compound of formula (26) wherein R is C₁-C₁₀ alkyl or C₃-C₁₀ cycloalkyl, and pharmaceutically acceptable salts thereof, which comprises:
a) adding a cyanoacetate of formula 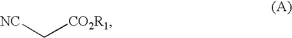
   wherein R₁ is selected from methyl, ethyl, n-propyl, iso-propyl, n-butyl, iso-butyl, sec-butyl, tert-butyl, and benzyl, to a mixture of a chiral cyclopentanone of formula 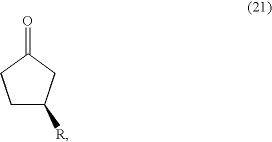
   a solvent selected from tetrahydrofuran, 1,4-dioxane, tert-butylmethylether, chloroform, dichloromethane, acetonitrile, ethyl ether, ethyl acetate, hexanes, N,N-dimethylformamide, dimethylsulfoxide, ethanol, tert-butanol, toluene, benzene, xylenes, and n-heptane, acetic acid, and a Knoevenagel reaction catalyst selected from β-alanine, ammonium acetate, and piperidine, and stirring the mixture in the presence of a means of removing water selected from azeotropic distillation, activated molecular sieves, anhydrous magnesium sulfate, anhydrous cesium carbonate, trimethyl orthoformate, and triethyl orthoformate to produce the alkene of formula 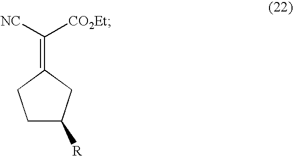
b) adding the product of Step a) above to a mixture of benzylmagnesium chloride, benzylmagnesium bromide, or benzylmagnesium iodide in a solvent selected from tetrahydrofuran, 1,4-dioxane, hexanes, n-heptane, toluene, diethyl ether, and tert-butyl methyl ether to produce the addition products of formulas 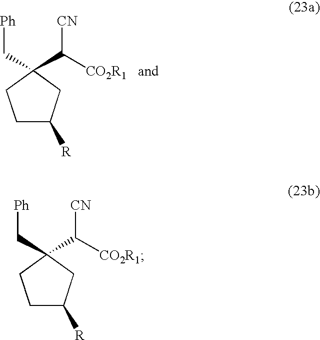
c) adding the products of Step b) above to a mixture of a base selected from potassium hydroxide, sodium hydroxide, lithium hydroxide, and cesium hydroxide in a solvent selected from ethylene glycol, 2-methoxyethyl ether, 1,4-dioxane, and diethylene glycol, and stirring the mixture, and then acidifying to produce the carboxylic acids of formulas 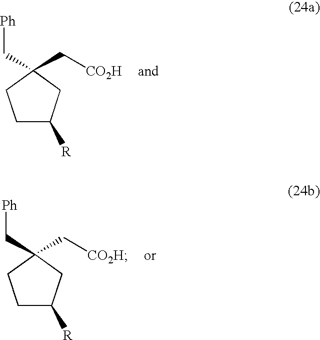
   adding the products of Step b) above to an acid mixture selected from 6-12 M HCl, 12 M H₂SO₄, 10%-48% wt/wt hydrobromic acid, and HBr in aqueous acetic acid, and stirring to produce the carboxylic acids of formulas 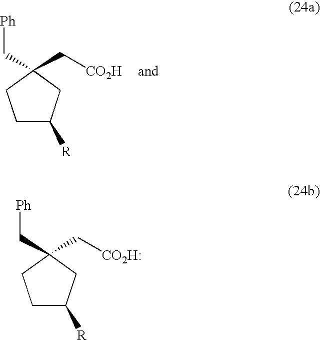
d) contacting the products of Step c) above with an amine selected from (S)-α-methyl-benzylamine, (R)-α-methyl-benzylamine, (R)-(+)-1-(naphthyl)ethylamine, (S)-(+)-1-(naphthyl)ethylamine, triethylamine, diisopropylethylamine, dicyclohexylamine, benzylamine, dibenzylamine, morpholine, N-methylmorpholine, piperidine, N-methylpiperidine, and pyridine in a solvent selected from N,N-dimethylformamide, chloroform, hexanes, acetone, ethanol, methanol, iso-propanol, diethyl ether, dichloromethane, benzene, toluene, n-pentane, n-hexane, n-heptane, ethyl acetate, acetonitrile, tert-butyl methyl ether, tetrahydrofuran, and 1,4-dioxane, and recrystallizing the salt so formed to produce the enriched diastereomer of formula 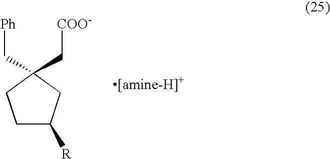
   as the amine salt, and
e) adding the product of Step d) to a mixture selected from aqueous hydrochloric acid, aqueous sulfuric acid, aqueous acetic acid, hydrochloric acid dissolved in acetic acid, and hydrochloric acid dissolved in acetic acid and water, and stirring to produce the carboxylic acid of formula 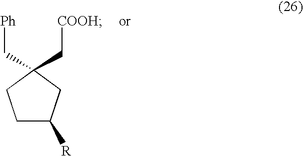
   partitioning the product of Step d) between a mixture of aqueous hydrochloric acid and a solvent selected from chloroform, dichloromethane, ethyl acetate, ethyl ether, tetrahydrofuran, 1,4-dioxane, toluene, and tert-butylmethylether, and drying and evaporating the organic layer to produce the carboxylic acid of formula

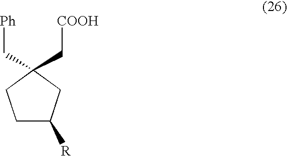

More preferred is a process for the preparation of a compound of formula (26) wherein R is C₁-C₁₀ alkyl or C₃-C₁₀ cycloalkyl, and pharmaceutically acceptable salts thereof, which comprises:
a) adding a cyanoacetate of formula 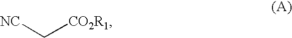
   wherein R₁ is ethyl, to a mixture of a chiral cyclopentanone of formula 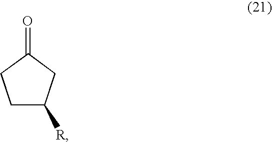
   toluene, acetic acid, and a Knoevenagel reaction catalyst which is ammonium acetate, and heating the mixture at reflux over a Dean-Stark trap to produce the alkene of formula 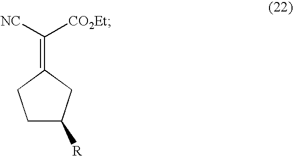
b) adding the product of Step a) above to a mixture of benzylmagnesium chloride in dry tetrahydrofuran at −100° C. to −20° C. to produce the addition products of formulas 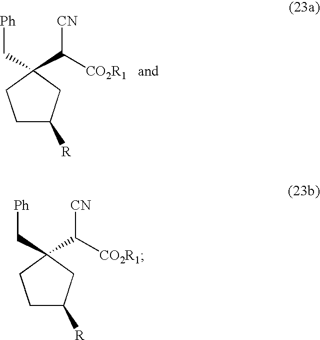
c) adding the products of Step b) above to a mixture of potassium hydroxide in ethylene glycol, and heating the mixture at 100° C. to 200° C., and then acidifying to produce the hydrolysis products of formulas 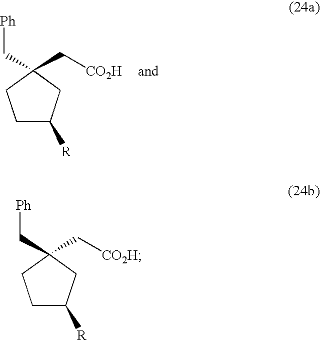
d) contacting the products of Step c) above with (R)-α-methyl-benzylamine in ethyl acetate, and recrystallizing the salt so formed from ethyl acetate to produce the enriched diastereomer of formula 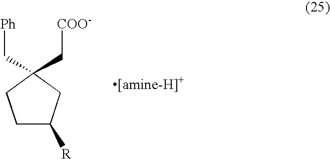
   as the (R)-α-methyl-benzylamine salt; and
e) adding the product of Step d) to aqueous hydrochloric acid and stirring to produce the carboxylic acid of formula

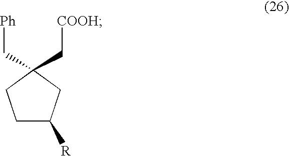

Further, the invention provides a key intermediate of formula (6) 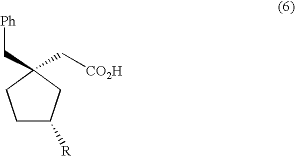
wherein R is C₁-C₁₀ alkyl or C₃-C₁₀ cycloalkyl, and pharmaceutically acceptable salts thereof.

Preferred is a compound of formula (6) and pharmaceutically acceptable salts thereof wherein R is C₁-C₁₀ alkyl.

More preferred is a compound of formula (6) and pharmaceutically acceptable salts thereof wherein R is selected from methyl, ethyl, and n-propyl,

Still more preferred is a compound of formula (6) named ((1S,3R)-1-benzyl-3-methyl-cyclopentyl)-acetic acid.

Further, the invention provides a key intermediate of formula (26) 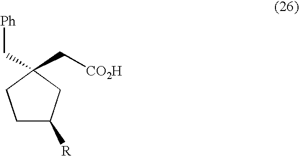
wherein R is C₁-C₁₀ alkyl or C₃-C₁₀ cycloalkyl and pharmaceutically acceptable salts thereof.

Preferred is a compound of formula (26) and pharmaceutically acceptable salts thereof wherein R is C₁-C₁₀ alkyl.

More preferred is a compound of formula (26) and pharmaceutically acceptable salts thereof wherein R is selected from methyl, ethyl, and n-propyl,

Still more preferred is a compound of formula (26) named ((1R,3S)-1-benzyl-3-methyl-cyclopentyl)-acetic acid.

Further, the invention provides a compound of Formula I, wherein R is as defined above, prepared according to any one of the processes for the preparation of a compound of Formula I described above.

Preferred is a compound of Formula I, wherein R is C₁-C₁₀ alkyl, prepared according to any one of the processes for the preparation of a compound of Formula I described above.

More preferred is a compound of Formula I, wherein R is selected from methyl, ethyl, and n-propyl, prepared according to any one of the processes for the preparation of a compound of Formula I described above.

Still more preferred is a compound of Formula I selected from:

((1R,3S)-1-aminomethyl-3 -methyl-cyclopentyl)-acetic acid; and

((1R,3S)-1-aminomethyl-3-methyl-cyclopentyl)-acetic acid hydrochloride, prepared according to any one of the processes for the preparation of a compound of Formula I described above.

Further, the invention provides a compound of Formula I, wherein R is as defined above, prepared according to any one of the processes for the preparation of a compound of Formula II described above.

Preferred is a compound of Formula II, wherein R is C₁-C₁₀ alkyl, prepared according to any one of the processes for the preparation of a compound of Formula II described above.

More preferred is a compound of Formula II, wherein R is selected from methyl, ethyl, and n-propyl, prepared according to any one of the processes for the preparation of a compound of Formula II described above.

Still more preferred is a compound of Formula II selected from:

((1S,3R)-1-aminomethyl-3-methyl-cyclopentyl)-acetic acid; and

((1S,3R)-1-aminomethyl-3-methyl-cyclopentyl)-acetic acid hydrochloride, prepared according to any one of the processes for the preparation of a compound of Formula II described above.

Further, the invention provides a compound of Formula III, wherein R is as defined above, prepared according to any one of the processes for the preparation of a compound of Formula III described above.

Further, the invention provides a compound of Formula IV, wherein R is as defined above, prepared according to any one of the processes for the preparation of a compound of Formula IV described above.

Further, the invention provides a compound of formula (6), wherein R is as defined above, prepared according to any one of the processes for the preparation of a compound of formula (6) described above.

Preferred is a compound of formula (6), wherein R is C₁-C₁₀ alkyl prepared according to any one of the processes for the preparation of a compound of formula (6) described above.

More preferred is a compound of formula (6), wherein R is selected from methyl, ethyl, and n-propyl, prepared according to any one of the processes for the preparation of a compound of formula (6) described above.

Still more preferred is a compound of formula (6) named ((1S,3R)-1-benzyl-3-methyl-cyclopentyl)-acetic acid, prepared according to any one of the processes for the preparation of a compound of formula (6) described above.

Further, the invention provides a compound of formula (26), wherein R is as defined above, prepared according to any one of the processes for the preparation of a compound of formula (26) described above.

Preferred is a compound of formula (26), wherein R is C₁-C₁₀ alkyl, prepared according to any one of the processes for the preparation of a compound of formula (26) described above.

More preferred is a compound of formula (26), wherein R is selected from methyl, ethyl, and n-propyl, prepared according to any one of the processes for the preparation of a compound of formula (26) described above.

Still more preferred is a compound of formula (26) named

((1R,3S)-1-benzyl-3-methyl-cyclopentyl)-acetic acid, prepared according to any one of the processes for the preparation of a compound of formula (26) described above.

Further, the invention provides a compound of Formula I selected from

((1R,3R)-1-aminomethyl-3-methyl-cyclopentyl)-acetic acid;

((1R,3R)-1-aminomethyl-3-methyl-cyclopentyl)-acetic acid hydrochloride;

((1R,3R)-1-aminomethyl-3-ethyl-cyclopentyl)-acetic acid;

((1R,3R)-1-aminomethyl-3-ethyl-cyclopentyl)-acetic acid hydrochloride;

((1R,3R)-1-aminomethyl-3-propyl-cyclopentyl)-acetic acid; and

((1R,3R)-1-aminomethyl-3-propyl-cyclopentyl)-acetic acid hydrochloride.

Further, the invention provides a compound of Formula II selected from:

((1S,3R)-1-aminomethyl-3-methyl-cyclopentyl)-acetic acid;

((1S,3R)-1-aminomethyl-3 -methyl-cyclopentyl)-acetic acid hydrochloride;

((1S,3R)-1-aminomethyl-3-ethyl-cyclopentyl)-acetic acid;

((1S,3R)-1-aminomethyl-3-ethyl-cyclopentyl)-acetic acid hydrochloride;

((1S,3R)-1-aminomethyl-3-propyl-cyclopentyl)-acetic acid; and

((1S,3R)-1-aminomethyl-3-propyl-cyclopentyl)-acetic acid hydrochloride;

Further, the invention provides compounds selected from:

E-Cyano-((R)-3-methyl-cyclopentylidene)-acetic acid ethyl ester;

Z-Cyano-((R)-3-methyl-cyclopentylidene)-acetic acid ethyl ester;

(R)-((1S,3R)-1-Benzyl-3 -methyl-cyclopentyl)-cyano-acetic acid ethyl ester;

(S)-((1S,3R)-1-Benzyl-3-methyl-cyclopentyl)-cyano-acetic acid ethyl ester;

(R)-((1R,3R)-1-Benzyl-3-methyl-cyclopentyl)-cyano-acetic acid ethyl ester;

(S)-((1R,3R)-1-Benzyl-3-methyl-cyclopentyl)-cyano-acetic acid ethyl ester;

((1S,3R)-1-Isocyanatomethyl-3-methyl-cyclopentylmethyl)-benzene;

((1S,3R)-1-Benzyl-3-methyl-cyclopentylmethyl)-carbamic acid methyl ester;

[(1S,3R)-1-(Methoxycarbonylamino-methyl)-3-methyl-cyclopentyl]-acetic acid;

((1S,3R)-1-Benzyl-3-methyl-cyclopentyl)-acetic acid methyl ester;

(1S,3R)-1-Methoxycarbonylmethyl-3 -methyl-cyclopentyl)-acetic acid;

((1R,3R)-1-Isocyanatomethyl-3-methyl-cyclopentyl)-acetic acid methyl ester;

[(1R,3R)-1-(Methoxycarbonylamino-methyl)-3-methyl-cyclopentyl]-acetic acid methyl ester;

((1S,3R)-1-Benzyl-3-methyl-cyclopentyl)-acetic acid tert-butyl ester;

[(1S,3R)-1-Carboxymethyl-3-methyl-cyclopentyl]-acetic acid tert-butyl ester;

[(1S,3R)-1-Methoxycarbonylmethyl-3-methyl-cyclopentyl]-acetic acid tert-butyl ester;

((1R,3R)-1-Methoxycarbonylmethyl-3-methyl-cyclopentyl)-acetic acid;

((1S,3R)-1-Isocyanatomethyl-3 -methyl-cyclopentyl)-acetic acid methyl ester; and

[(1S,3R)-1-(Methoxycarbonylamino-methyl)-3-methyl-cyclopentyl]-acetic acid methyl ester.

More preferred is a process for the preparation of a compound of formula 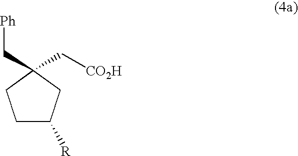
wherein R is C₁-C₁₀ alkyl or C₃-C₁₀ cycloalkyl, comprising, hydrolyzing a compound of formula 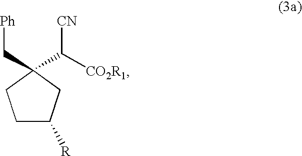
wherein R₁ is H, alkyl, or benzyl.

More preferred is a process for the preparation of a compound of formula 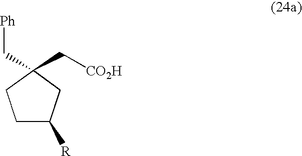
wherein R is C₁-C₁₀ alkyl or C₃-C₁₀ cycloalkyl, comprising, hydrolyzing a compound of formula 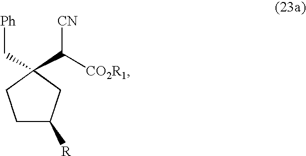
wherein R₁ is H, alkyl, or benzyl.

More preferred is a process for the preparation of a compound of formula 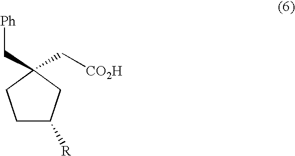
wherein R is C₁-C₁₀ alkyl or C₃-C₁₀ cycloalkyl, comprising, resolving a mixture containing compounds of formulas 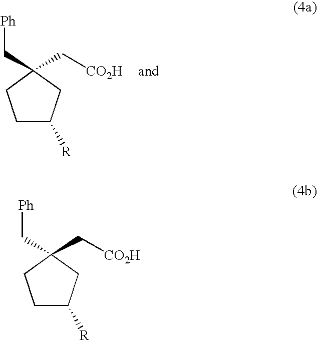
wherein R is C₁-C₁₀ alkyl or C₃-C₁₀ cycloalkyl.

More preferred is a process for the preparation of a compound of formula 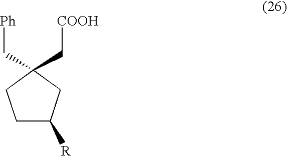
wherein R is C₁-C₁₀ alkyl or C₃-C₁₀ cycloalkyl, comprising, resolving a mixture containing compounds of formulas 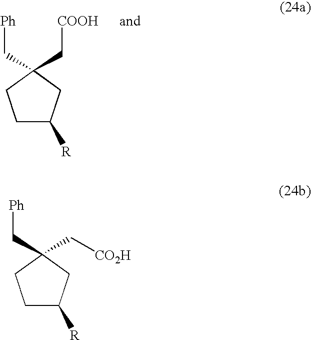
wherein R is C₁-C₁₀ alkyl or C₃-C₁₀ cycloalkyl.

More preferred is a process for the preparation of a compound of Formula I 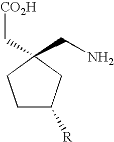
wherein R is C₁-C₁₀ alkyl or C₃-C₁₀ cycloalkyl, and pharmaceutically acceptable salts thereof, comprising, hydrolyzing a compound of formula 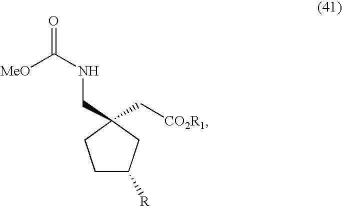
wherein R₁ is H, alkyl, or benzyl, and contacting the product, if desired, with an acid or a base.

More preferred is a process for the preparation of a compound of Formula II 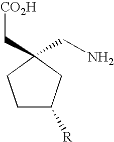
wherein R is C₁-C₁₀ alkyl or C₃-C₁₀ cycloalkyl, and pharmaceutically acceptable salts thereof, comprising hydrolyzing a compound of formula 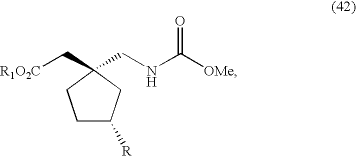
wherein R₁ is H, alkyl or benzyl, and contacting the product, if desired, with an acid or a base,

More preferred is a process for the preparation of a compound of Formula III 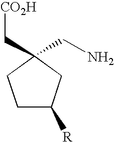
wherein R is C₁-C₁₀ alkyl or C₃-C₁₀ cycloalkyl, and pharmaceutically acceptable salts thereof, comprising, hydrolyzing a compound of formula 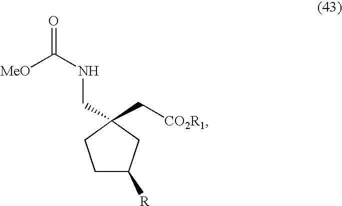
wherein R₁ is H, alkyl, or benzyl, and contacting the product, if desired, with an acid or a base.

More preferred is a process for the preparation of a compound of Formula IV 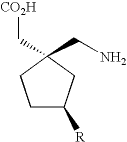
wherein R is C₁-C₁₀ alkyl or C₃-C₁₀ cycloalkyl, and pharmaceutically acceptable salts thereof, comprising, hydrolyzing a compound of formula 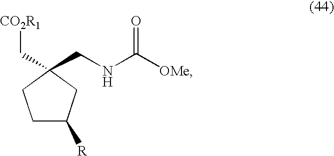
wherein R₁ is H, alkyl, or benzyl, and contacting the product, if desired, with an acid or a base.

### DETAILED DESCRIPTION OF THE INVENTION

The instant invention is an important process as it permits the synthesis of single isomers; it is a route to stereospecific 3-substituted 5-membered rings of formula

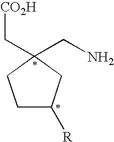

A key feature of the invention is the stereoselective preparation of a compound of formula (6) by selective fractional crystallization of a salt of formula (5) from a mixture of compounds of formulas (4a) and (4b), and conversion of a salt of formula (5) to a compound of formula (6). Another feature of the invention is the conversion of a compound of formula (2) to a mixture of compounds of formulas (3a) and (3b) wherein the yield of a compound of formula (3a) over a diastereomer of formula (3b) may be enhanced by optimizing certain reaction parameters such as, for example, temperature. Reaction of a compound of formula (2) at a relatively low temperature generally provides for a relatively higher yield of a compound of formula (3a) over (3b) than when the reaction is run at a higher temperature. The invention also provides for translation of the stereochemistry at the two chiral carbons of the cyclopentane ring of the resulting pure enantiomer of formula (6) into enantiomerically pure compounds of Formulas I or II with little or no racemization.

Another key feature of the invention is the stereoselective preparation of a compound of formula (26) by selective fractional crystallization of salt of formula (25) from a mixture of compounds of formulas (24a) and (24b), and conversion of a salt of formula (25) to a compound of formula (26). Another feature of the invention is the conversion of a compound of formula (22) to a mixture of compounds of formulas (23a) and (23b) wherein the yield of a compound of formula (23a) over a diastereomer of formula (23b) may be enhanced by optimizing certain reaction parameters such as, for example, temperature. Reaction of a compound of formula (22) at a relatively low temperature generally provides for a relatively higher yield of a compound of formula (23a) over (23b) than when the reaction is run at a higher temperature. The invention also provides for translation of the stereochemistry at the two chiral carbons of the cyclopentane ring of the resulting pure enantiomer of formula (26) into enantiomerically pure compounds of Formulas III or IV with little or no racemization.

The final products are useful as agents in the treatment of epilepsy, faintness attacks, hypokinesia, cranial disorders, neurodegenerative disorders depression, anxiety, panic, pain, neuropathological disorders, gastrointestinal disorders such as irritable bowel syndrome (IBS), inflammation especially arthritis, sleep disorders, premenstrual syndrome, and hot flashes.

The following experimental procedures provide a novel route to be used to stereoselectively synthesize 3-substituted cyclopentyl-based analogs of gabapentin and pharmaceutically acceptable salts thereof. These routes provide access to pure stereoisomers of Formulas I, II, III, and IV 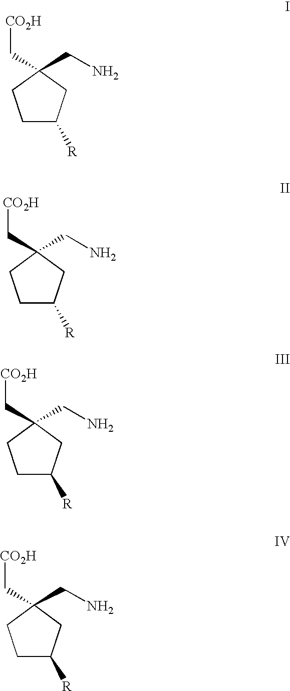
wherein R is C₁-C₁₀ alkyl or C₃-C₁₀ cycloalkyl.

Examples 1 and 3 below each show a synthesis of a compound of Formula II wherein R is methyl.

Example 2 below shows a synthesis of a compound of Formula I wherein R is methyl.

It is understood that compounds of Formulas I, II, III, or IV, or a pharmaceutically acceptable salt thereof, produced by a hydrolysis reaction such as, for example, step j) in the above process for the preparation of a compound of Formula I, or a pharmaceutically acceptable salt thereof, or the process described above wherein a compound of formula (41) is hydrolyzed, may be formed as an acid or base salt thereof, which salt may be optionally converted to a free amino acid form or a pharmaceutically acceptable salt form thereof by methods well known to a skilled person in the pharmaceutical or chemical arts.

The following terms are defined as used herein.

As used herein the term “C₁-C₁₀ alkyl” means a straight or branched alkyl group or radical containing from 1 to 10 carbon atoms. Illustrative examples of C₁-C₁₀ alkyl include methyl, ethyl, 1-propyl, 2-propyl, 1-butyl, 2-butyl, 2-methyl-1-propyl, 1,1-dimethylethyl, 1-pentyl, 2-pentyl, 3-pentyl, 2,2-dimethylpropyl, 1-hexyl, 2-hexyl, 3-hexyl, 4-methyl-1-pentyl, 1-heptyl, 2-heptyl, 3-heptyl, 4-heptyl, 5-methyl-1-hexyl, 1-octyl, 2-octyl, 3-octyl, 4-octyl, 6-methyl-1-heptyl, 5,5-dimethylhexyl, 1-nonyl, 2-nonyl, 1-decyl, and 2-decyl.

The term “C₃-C₁₀ cycloalkyl” means a cycloalkyl group or radical having from 3 to 10 carbon atoms. Illustrative examples of a C₃-C₁₀ cycloalkyl include cyclopropyl, cyclobutyl, cyclopentyl, cyclohexyl, cycloheptyl, cyclooctyl, cyclononyl and cyclodecyl.

The term “stereoisomer” means any of a group of isomers in which identical atoms are linked in the same order but differ in their spatial arrangement.

| | |
|---|---|
| * | Asterisk symbol points out an |
| | enantiomerically enriched chiral carbon |
| | atom |
| AcOH | Acetic acid |
| Alkali hydroxide | LiOH, NaOR, KOH, or CsOH |
| NH₄OAc | Ammonium acetate |
| BnMgCl or PhCH₂MgCl | Benzylmagnesium chloride |
| t-BuOH or tert-butyl | 1,1-Dimethylethanol |
| alcohol | |
| ^{t}Butyl | 1,1-Dimethylethyl |
| CH₂Cl₂ | Dichloromethane |
| CCl₄ | Carbon tetrachloride |
| CDCl₃ | Deuterochloroform |
| (CH₃)₃SiCHN₂ | Trimethylsilyldiazomethane |
| CN | Carbon-nitrogen triple bond (nitrile) |
| (COCl)₂ | Oxalyl chloride |
| CsOH | Cesium hydroxide |
| de | Diastereomeric excess |
| DBU | 1,8-Diazabicyclo[5.4.0]undec-7-ene |
| DMF | N,N-Dimethylformamide |
| DMSO | Dimethylsulfoxide |
| DMSO-d₆ | Deuterated dimethylsulfoxide |
| ee | Enantiomeric excess |
| Et | Ethyl |
| EtOAc | Ethyl acetate |
| Et₃N | Triethylamine |
| HCl | Hydrogen chloride |
| HCl (aq) | Hydrochloric acid |
| 6N HCl | 6 normal hydrochloric acid |
| HCl (g) | Hydrogen chloride (gaseous) |
| ¹H-NMR | Proton (nuclear) magnetic resonance |
| | spectroscopy |
| IR | Infrared spectroscopy |
| J | Coupling constant in Hz |
| KOH | Potassium hydroxide |
| LCMS | Liquid chiomatography-mass spectrometry |
| LiOH | Lithium hydroxide |
| Me | Methyl |
| MeO | Methoxy |
| MeCN | Acetonitrile |
| MeI | Iodomethane |
| MeOH | Methanol |
| MgSO₄ | Magnesium sulfate |
| MS (ES⁺) | Positive ion electrospray mass spectrometry |
| MS (ES⁻) | Negative ion electrospray mass spectrometry |
| MS (CI⁺) | Positive ion chemical ionization mass |
| | spectrometry |
| MS (CI⁻) | Negative ion chemical ionization mass |
| | spectrometry |
| m/z | mass per unit charge |
| NCCH₂CO₂Et | Ethyl cyanoacetate |
| NaIO₄ | Sodium periodate |
| NaOH | Sodium hydroxide |
| ODS | Octadecyl-functionalized silica gel |
| Ph | Phenyl |
| (i-Pr)₂NEt | Diisopropylethylamine |
| R_{f} | R_{f} value |
| RuCl₃ | Ruthenium(III) chloride |
| SOCl₂ | Thionyl chloride |
| TFA or CF₃CO₂H | Trifluoroacetic acid |
| THF | Tetrahydrofuran |

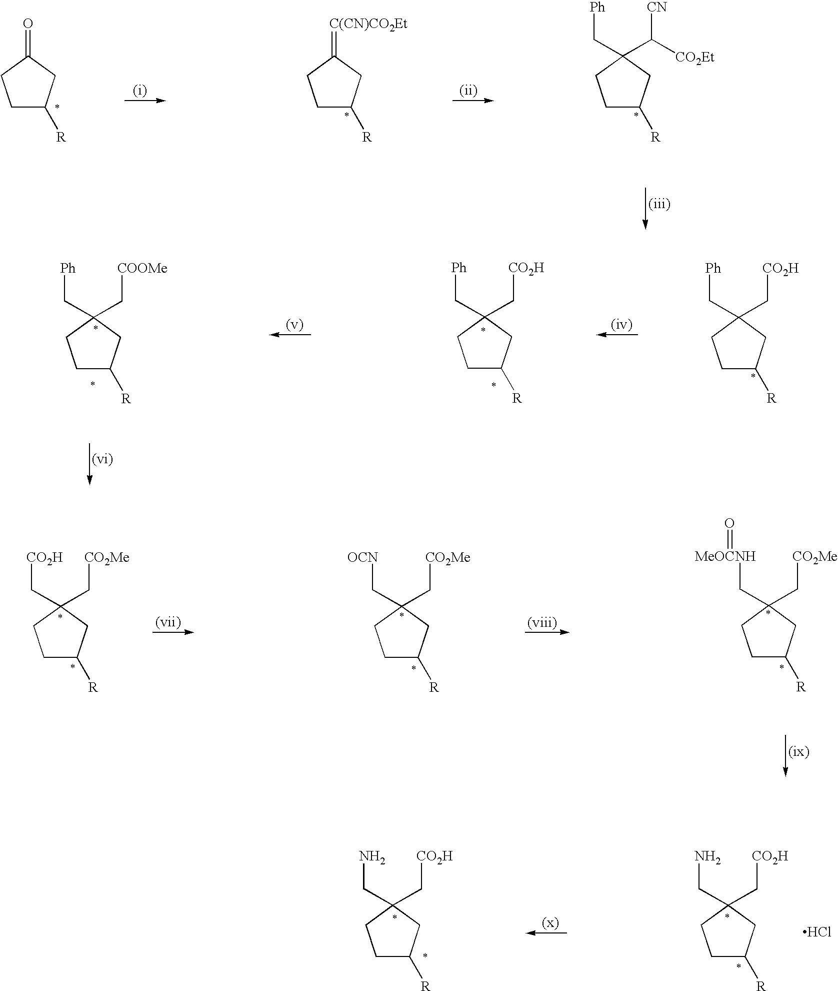

Reagents and Conditions:
(i) NCCH₂CO₂Et, catalyst (e.g., NH₄OAc, ACOH):
(ii) BnMgCl;
(iii) hydrolysis using, for example, alkali hydroxide (e.g. KOH);
(iv) a) resolution using a resolving agent (e.g., (R)— or (S)-α-methylbenzylamine);
   b) conversion of the enriched stereoisomer to the free acid using, for example, hydrochloric acid;
(v) esterification using, for example, Mel and DBU;
(vi) oxidation using, for example, RuCl₃ and NaIO₄;
(vii) (PhO)₂P(O)N₃ and a base (e.g., Et₃N);
(viii) MeOH;
(ix) hydrolysis using HCl (aq);
(x) conversion to the free amino acid using, for example, H₂O and alkali hydroxide (e.g., NaOH).

### General Route B

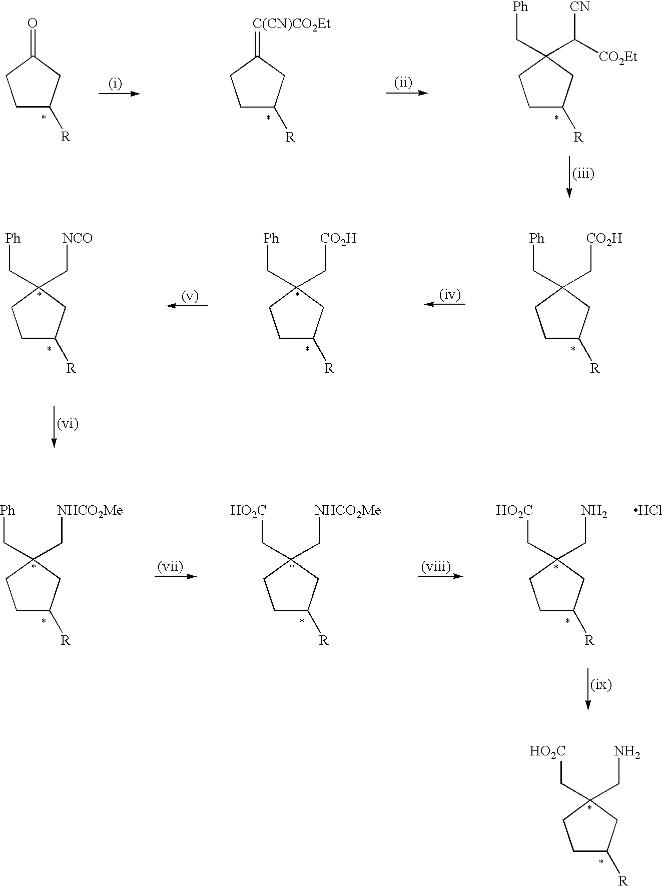

Reagents and Conditions:
(i) NCCH₂CO₂Et, catalyst (e.g. NH₄OAc, ACOH):
(ii) BnMgCl:
(iii) hydrolysis using for example, alkali hydroxide (e.g. KOH);
(iv) a) resolution using a resolving agent (e.g., (R)— or (S)-α-methylbenzylamine);
   b) conversion of salt of enriched stereoisomer to the free acid using for example, hydrochloric acid;
(v) (PhO)₂P(O)N₃ and base (e.g., Et₃N);
(vi) MeOH;
(vii) oxidation using, for example, RuCl₃ and NaIO₄;
(viii) hydrolysis using HCl (aq);
(ix) conversion to the free amino acid using, for example, H₂O and alkali hydroxide (e.g., NaOH).

### General Route C

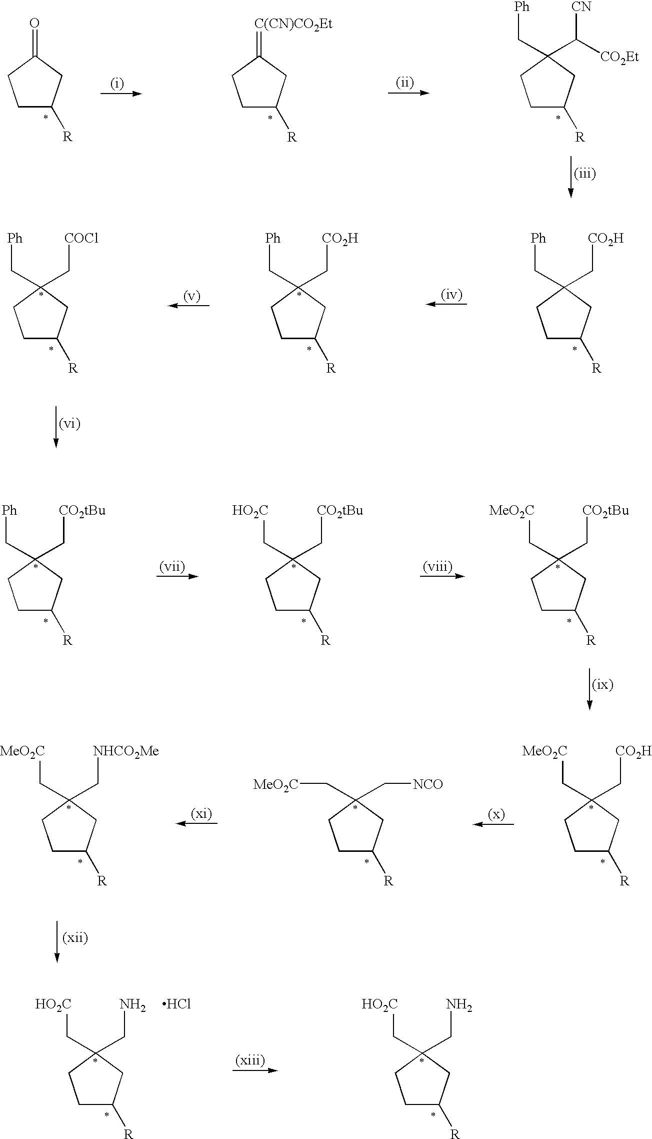

Reagents and Conditions:
(i) NCCH₂CO₂Et, catalyst (e.g., NH₄OAc, ACOH);
(ii) BnMgCl;
(iii) hydrolysis using, for example, alkali hydroxide (e.g., KOH);
(iv) a) resolution using a resolving agent (e.g., (R)— or (S)-α-methylbenzylamine);
   b) conversion of salt of enriched stereoisomer to the free acid using, for example, hydrochloric acid;
(v) chlorination using, for example, (COCl)₂ or SOCl₂;
(vi) tBuOH and base (e.g., Et₃N);
(vii) oxidation using, for example, RuCl₃ and NaIO₄;
(viii) esterification using, for example, (CH₃)₃SiCHN₂ and MeOH;
(ix) dealkylation using, for example, CF₃CO₂H;
(x) (PhO)₂P(O)N₃ and a base (e.g., Et₃N);
(xi) MeOH;
(xii) hydrolysis using HCl (aq);
(xiii) conversion to the free amino acid using, for example, H₂O and alkali hydroxide (e.g., NaOH).

### EXAMPLE 1

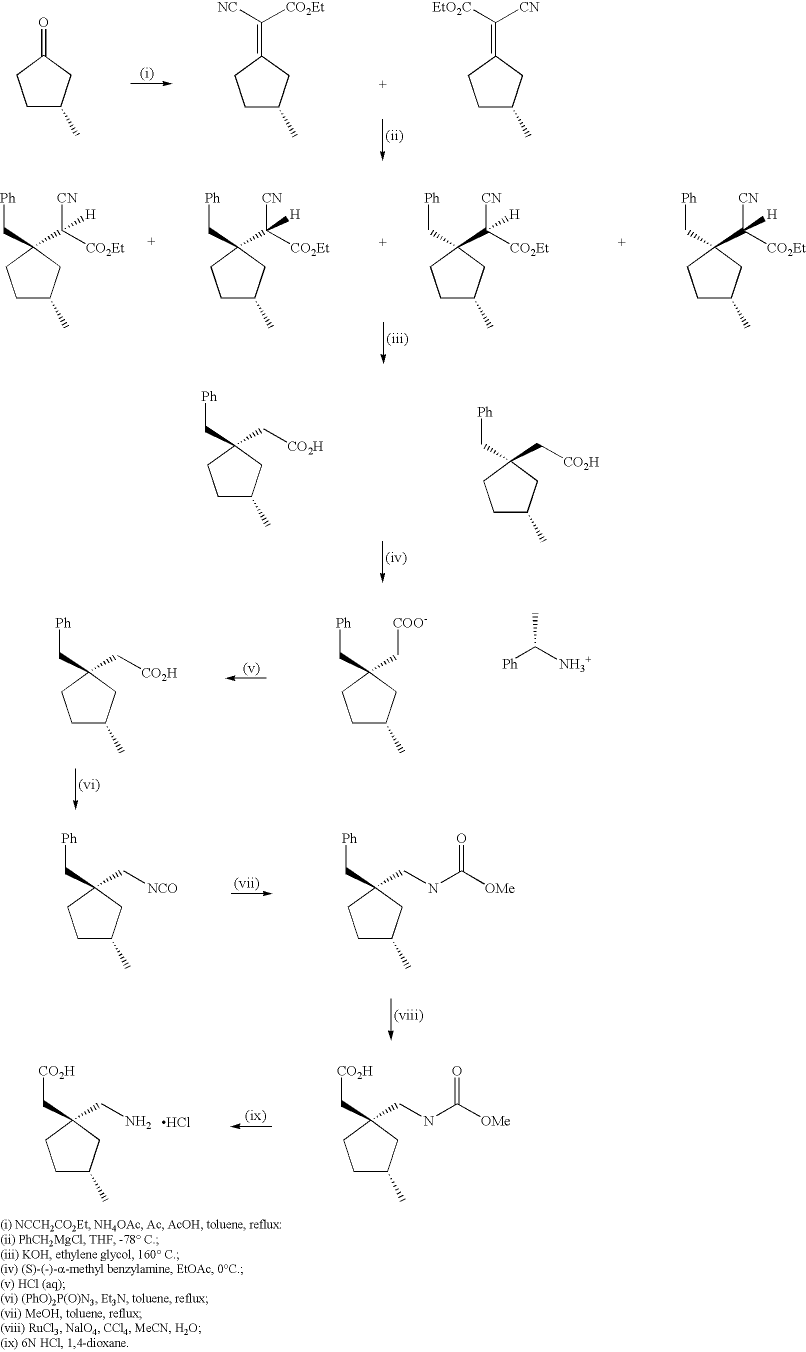

### (E and Z)-Cyano-((R)-3-methyl-cyclopentylidene)-acetic acid ethyl ester

(R)-(+)-3-Methylcyclopentanone (5 g, 51.0 mmol), ethyl cyanoacetate (5.42 mL, 51.0 mmol), ammonium acetate (0.4 g, 5.1 mmol), and glacial acetic acid (0.58 mL. 10.2 mmol) were refluxed in toluene (30 mL) using a Dean-Stark trap. After 6 hours, the mixture was allowed to cool and diluted with ethyl acetate (100 mL), washed with water (3×80 mL), brine, and dried (MgSO₄). The solvent was evaporated under reduced pressure. The residue was chromatographed (silica gel, heptane/ethyl acetate, 9:1) to give 8.87 g (90%) of a 1:1 mixture of (E and Z)-cyano-((R)-3-methyl-cyclopentylidene)-acetic acid ethyl ester;

R_{f} (heptane-ethyl acetate, 9:1) 0.28:

IR thin film (cm⁻¹) 2225 (CN), 1724 (C═O), 1617 (C═C);

¹H-NMR (400 MHz; CDCl₃): δ 4.27 (2H, q, J 7.2. CO₂CH₂Me), 4.26 (2H q, J 7.2. CO₂CH₂Me), 3.35 (1H, dt, J 7.1. 1.6), 3.30 (1H, dt, J 7.1. 1.6), 3.23 (1H, ddd, J 8.1. 3.5, 1.7), 3.18 (1H, ddd, J 8.1, 3.4, 1.7), 3.05-2.67 (4H, m). 2.50-2.32 (2H m), 2.29-1.96.(4H, m), 1.50-1.35 (2H, m), 1.34 (3H, t, J 7.2 CO₂CH₂Me), 1.33 (3H, t, J 7.1. CO₂CH₂Me), 1.10 (3H, d, J 6.6 Me), 1.08 (3H, d, J 6.6, Me);

MS (ES⁻): m/z 192 (M−H, 100%).

### (R and S)-((1S,3R)-1-Benzyl-3-methyl-cyclopentyl)-cyano-acetic acid ethyl ester and (R and S)-((1R,3R)-1-Benzyl-3-methyl-cyclopentyl)-cyano-acetic acid ethyl ester

A mixture of (E and Z)-cyano-((R)-3-methyl-cyclopentylidene)-acetic acid ethyl ester (4.13 g, 21.4 mmol) in THY (30 mL) was added over 1 hour to a stirring solution of benzylmagnesium chloride (27.7 mL of a 1 M solution in ether, 27.7 mmol) in THF (50 mL) at −78° C. under argon. After stirring for a further 1 hour, the mixture was quenched by addition of saturated ammonium chloride solution (15 mL). The mixture was allowed to warm to room temperature, diluted with ether (30 mL), and dilute hydrochloric acid (20 mL) was added. The organic layer was separated, and the aqueous layer was further extracted with ether (2>40 mL). The combined ether layers were washed with brine, dried (MgSO₄), and the solvent was evaporated under reduced pressure. The residue was chromatographed (silica gel, heptane-ethyl acetate, 95:5) to give 5.8 g (100%) of a 7:7:3:3 mixture of diastereomeric (R and S)-((1S,3R)-1-benzyl-3-methyl-cyclopentyl)-cyano-acetic acid ethyl ester and (R and S)-((1R,3R)-1-benzyl-3-methyl-cyclopentyl)-cyano-acetic acid ethyl ester

R_{f} (heptane-ethyl acetate, 9:1) 0.32;

IR thin film (cm⁻¹) 2246 (CN), 1740 (C═O), 1603 (C═C);

MS (ES⁻) m/z 284 (M−H, 100%).

### ((1S,3R)-1-Benzyl-3-methyl-cyclopentyl)-acetic acid and ((1R,3R)-1-Benzyl-3-methyl-cyclopentyl)-acetic acid

The mixture of (R and S)-((1S,3R)-1-benzyl-3-methyl-cyclopentyl)-cyano-acetic acid ethyl ester and (R and S)-((1R,3R)-1-benzyl-3-methyl-cyclopentyl)-cyano-acetic acid ethyl ester (1 g, 3.5 mmol) and potassium hydroxide (1.2 g, 21.4 mmol) were heated to 160° C. in ethylene glycol (5 mL) for 16 hours. After this time, the mixture was allowed to cool and dilute hydrochloric acid (150 mL) was added carefully. The mixture was extracted with ethyl acetate (3×50 mL) and the combined organic fractions were washed with brine, dried (MgSO₄), and the solvent was evaporated under reduced pressure. The residue was chromatographed (silica gel, heptane/ethyl acetate, 98:2) to give 0.65 g (80%) of a 7:3 mixture of ((1S,3R)-1-benzyl-3-methyl-cyclopentyl)-acetic acid and ((1R,3R)-1-benzyl-3-methyl-cyclopentyl)-acetic acid as an oil.

R_{f} (heptane-ethyl acetate, 98:2) 0.36;

IR thin film (cm⁻¹) 1702 (C═O);

¹H-NMR (400 MHz; CDCl₃) major isomer ((1S,3R)-1-benzyl-3-methyl-cyclopentyl)-acetic acid: δ 7.31-7.21 (5H, m, Ph), 2.82 (1H d, J 13.4, CH_{A}H_{B}CO₂H), 2.76 (1H, d, J 13.4, CH_{A}H_{B}CO₂H), 2.33 (2Hz br s, CH₂Ph), 2.19-1.66 (m), 1.62-1.52 (m), 1.11 (1H, dd, J 13.0, 9.9), 1.01 (3H, d, J 6.6, Me); minor isomer ((1R,3R)-1-benzyl-3-methyl-cyclopentyl)-acetic acid: δ 7.31-7.21 (5H, m, Ph), 2.89 (1H, d, J 13.2. CH_{A}H_{B}CO₂H), 2.84 (1H, d, J 13.4, CH_{A}H_{B}CO₂H), 2.28 (2H, br s, CH₂Ph), 2.19-1.66 (m), 1.62-1.52 (m), 1.30-1.17 (m), 1.00 (3H, d, J 6.6, Me);

MS (Cl⁻): m/z 231 (M−H. 100%).

### ((1S,3R)-1-Benzyl-3-methyl-cyclopentyl)-acetic acid

(s)-(−)-α-Methyl benzylamine (8.8 g, 72.7 mmol) was added to a stirring solution of the diastereomeric mixture of ((1S,3R)-1-benzyl-3-methyl-cyclopentyl)-acetic acid and ((1R,3R)-1-benzyl-3-methyl-cyclopentyl)-acetic acid (16.9 g, 72.7 mmol) dissolved in the minimum quantity of ethyl acetate. The mixture was placed in the fridge and left for 1 hour. After this time, the acid salt had crystallized out and this was filtered off. The salt was recrystallized several times from ethyl acetate (to 95% de). The salt was taken up in ethyl acetate, washed with dilute hydrochloric acid, brine and dried (MgSO₄). The solvent was evaporated under reduced pressure to give 6.8 g (40%) of ((1S,3R)-1-benzyl-3-methyl-cyclopentyl)-acetic acid; LCMS (Prodigy® (Phenomenex, Ltd.) ODS 3 50 mm×4.6 mm id column, 5-50% Acetonitrile/water) Retention Time=2.01 min. 98% purity.

### ((1S,3R)-1-Isocyanatomethyl-3-methyl-cyclopentylmethyl)-benzene

Diphenylphosphoryl azide (4.48 g, 16 mmol), triethylamine (1.69 g, 16.8 mmol), and acid ((1S,3R)-1-benzyl-3-methyl-cyclopentyl)-acetic acid (3.74 g, 16 mmol) were refluxed in toluene (40 mL) for 17 hours. The mixture was allowed to cool and then taken up in ethyl acetate (150 mL), washed with saturated aqueous sodium hydrogen carbonate (200 mL), brine (150 mL), and dried (MgSO₄). The solvent was removed under reduced pressure to give 3.69 g (100%) of ((1S,3R)-1-isocyanatomethyl-3-methyl-cyclopentylmethyl)-benzene, which was used without further purification;

R_{f} (heptane-ethyl acetate, 8:2) 0.36;

IR thin film (cm⁻¹) 2262 (CN).

### ((1S,3R)-1-Benzyl-3-methyl-cyclopentylmethyl)-carbamic acid methyl ester

((1S,3R)-1-Isocyanatomethyl-3-methyl-cyclopentylmethyl)-benzene (3.69 g, 16 mmol) was refluxed in methanol (10 mL) and toluene (20 mL) for 16 hours and then allowed to cool to room temperature. The solvent was removed under reduced pressure, and the residue was purified by chromatography (silica gel, heptane-ethyl acetate 9:1) to give 2.66 g (63%) of ((1S,3R)-1-benzyl-3-methyl-cyclopentylmethyl)-carbamic acid methyl ester.

R_{f} (heptane-ethyl acetate, 8:2) 0.28;

IR thin film (cm⁻¹) 1709 (C═O);

¹H-NMR (400 MHz; CDCl₃) δ 7.32-7.16 (5H, m, Ph), 4.60 (1H, bs, NH), 3.68 (3H, s, OMe), 3.18-3.00 (2H, m, CH₂NH), 2.62-2.60 (2H, s, CH₂Ph); 0.99 (3H, d, J 6.8. Me), 2.05-1.92, 1.87-1.72. 1.60-1.40. 1.00-0.89 (7H, m);

MS (ES⁺) m/z 262 (M+H, 90%), 302 (M+CH₃CN+H.100%);

LCMS (Prodigy® ODS 3 50 mm×4.6 mm id column. 5-50% Acetonitrile (0.05% formic acid)/water (0.05% formic acid)) Retention Time=2.11, 94% de.

### [(1S,3R)-1-(Methoxycarbonylamino-methyl)-3-methyl-cyclopentyl]-acetic acid

((1S,3R)-1-Benzyl-3-methyl-cyclopentylmethyl)-carbamic acid methyl ester (2.6 g, 9.9 mmol) and sodium periodate (29.8 g, 140 mmol) were stirred together in carbon tetrachloride (30 mL), acetonitrile (30 mL), and water for 6 hours. The mixture was cooled to 0° C., and ruthenium(II) chloride (0.04 g, 0.2 mmol) was added to the reaction mixture. The reaction was allowed to warm to room temperature and stirred for 20 hours. Diethyl ether (50 mL) was added, and the mixture was then extracted with saturated aqueous sodium hydrogen carbonate (200 mL). The aqueous layer was acidified to pH 1 with 4N hydrochloric acid and re-extracted with ethyl acetate (200 mL), dried (MeSO₄), and the solvent was evaporated under reduced pressure. The residue was purified by chromatography (silica gel, eluting with a gradient of heptane to 1:1 heptane:ethyl acetate) to give 0.32 g (14%) of [(1S,3R)-1-(methoxycarbonylamino-methyl)-3-methyl-cyclopentyl]-acetic acid

R_{f} (heptane-ethyl acetate, 8:2) 0.30;

IR thin film (cm⁻¹) 3338 (NH), 1712 (C═O);

¹H-NMR (400 MHz; CDCl₃): δ 9.29 (1H, s, COOH), 5.17 (1H, bs, NH), 3.71 (3H, s, OMe), 3.30 (1H, dd, J 14.4, 7.1, CH_{A}H_{B}NH₂), 3.17 (1H, dd, J 14.4, 6.6. CH_{A}H_{B}NH₂), 2.37 (2H, s, CH₂COOH), 2.20-1.00 (7H, m), 1.01 (3H, d, J 6.4, CHMe);

MS (ES⁺) m/z 230 (M+H, 63%). 481 (M+Na, 100).

### ((1S,3R)-1-Aminomethyl-3-methyl-cyclopentyl)-acetic acid hydrochloride

[(1S,3R)-1-(Methoxycarbonylamino-methyl)-3-methyl-cyclopentyl]-acetic acid (0.32 g, 1.4 mmol) was refluxed in a mixture of 1,4-dioxane (3 mL) and 6N Hydrochloric acid (8 mL) for 4 hours. The mixture was allowed to cool, diluted with water (200 mL), and washed with dichloromethane (2×200 mL). The aqueous layer was evaporated under reduced pressure, and the residue was recrystallized from ethyl acetate/methanol to give 0.17 g (59%) of ((1S,3R)-1-aminomethyl-3-methyl-cyclopentyl)-acetic acid hydrochloride;

IR thin film (cm⁻¹) 1710 (C═O):

¹H-NMR (400 MHz; DMSO-d₆): δ 2.96 (1H, d, J 12.8, CH_{A}H_{B}NH₂), 2.90 (1H, d, J 12.8, CH_{A}H_{B}NH₂), 2.40 (2H, s, CH₂COOH), 2.04 (1H, m, CHMe). 1.81-1.61, 1.51-1.43, 1.21-1.11 (5H, m), 1.06 (1H, dd. J 12.8. 10.4), 0.97 (3H, d, J 6.35, Me);

MS (ES⁺) m/z 173 (M+H, 100%), 196 (M+Na, 10%);

LCMS (Prodigy® ODS 3 50 mm×4.6 mm id column, 5% for 2 min, 5-50% over 1.5 min of Acetonitrile (0.05% formic acid)/water (0.05% formic acid)) Retention Time=0.92, 94% de.

### EXAMPLE 2

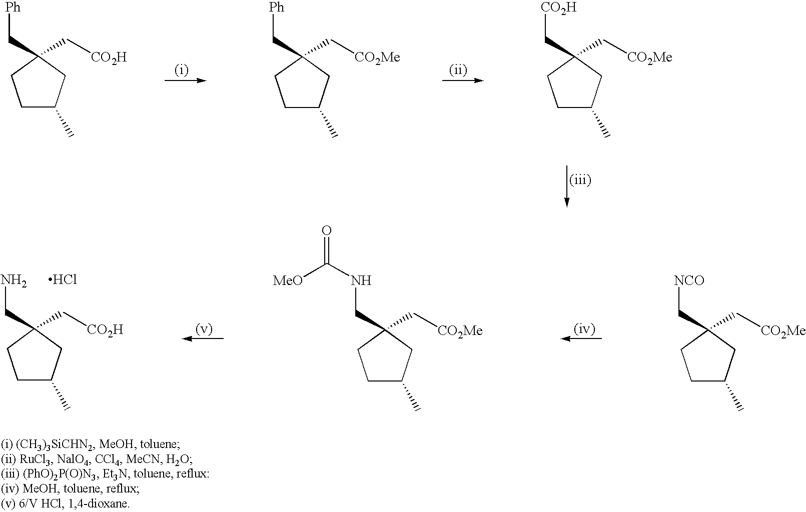

### ((1S,3R)-1-Benzyl-3-methyl-cyclopentyl)-acetic acid methyl ester

Trimethylsilyldiazomethane (31.5 mL of a 2 M solution in hexanes, 63 mmol) was added dropwise to a stirring solution of ((1S,3R)-1-benzyl-3-methyl-cyclopentyl)-acetic acid (10 g, 43 mmol) in toluene (80 mL) and methanol (20 mL) at 0° C. under argon, and the mixture was allowed to warm to room temperature. The mixture was stirred for 1 hour, and then the solvent was evaporated under reduced pressure. The residue was taken up in ethyl acetate (50 mL), washed with saturated sodium hydrogen carbonate solution, dilute hydrochloric acid, dried (MgSO₄), and the solvent removed in vacuo to give 10.6 g (100%) of ((1S,3R)-1-benzyl-3-methyl-cyclopentyl)-acetic acid methyl ester

R_{f} (heptane-ethyl acetate, 9:1) 0.40.

IR thin film (cm⁻¹) 1736 (C═O);

¹H-NMR (400 MHz, CDCl₃): δ 7.30-7.18 (5H, m, Ph), 3.69 (3H, s, OMe), 2.78 (1H, d, J 13.4, CH_{A}H_{B}CO₂Me), 2.72 (1H, d, J 13.4, CH_{A}H_{B}CO₂Me), 2.28 (2H, s, CH₂Ph), 2.16-1.50 (5H, m), 1.30-1.03 (2H, m), 1.00 (3H, d, J 6.6, Me).

### ((1S,3R)-1-Methoxycarbonylmethyl-3-methyl-cyclopentyl)-acetic acid

((1S,3R)-1-Benzyl-3-methyl-cyclopentyl)-acetic acid methyl ester (10.5 g, 43 mmol) and sodium periodate (128.0 g, 598 mmol) were stirred together in carbon tetrachloride (120 mL), acetonitrile (120 mL), and water (210 mL) for 1 hour. The mixture was cooled to 10° C., and ruthenium(III) chloride (0.177 g, 0.86 mmol) was added to the reaction mixture. The reaction was allowed to warm to room temperature and stirred for 20 hours. Diethyl ether (100 mL) was added, and the mixture was acidified to pH 1 with concentrated hydrochloric acid and then extracted with ether (2×200 mL). The organic layer was extracted with saturated aqueous sodium hydrogen carbonate (2×200 mL) which was then acidified to pH 1 with 4N hydrochloric acid and re-extracted with ethyl acetate, dried (MgSO₄), and concentrated in vacuo. The residue was purified by chromatography (silica gel, eluting with a gradient of heptane to 1:1 heptane:ethyl acetate) to give 8.02 g (87.7%) of ((1S,3R)-1-methoxycarbonylmethyl-3-methyl-cyclopentyl)-acetic acid;

R_{f} (heptane-ethyl acetate, 1:1) 0.46:

IR thin film (cm⁻¹) 3100 (OH), 1737 (C═O), 1705 (C═O),

¹H-NMR (400 MHz: CDCl₃): δ 3.68 (3H, s, OMe), 2.67-2.51 (4H, m), 2.06 (1H, m), 1.97-1.79 (2H, m), 1.76-1.59 (2H, m), 1.29-1.08 (2H, m), 1.01 (3H, d, J 6.6, Me);

MS (ES⁺) m/z 215 (M+H), 278 (M+Na, 100), 451 (2M+Na, 80%).

### ((1R,3R)-1-Isocyanatomethyl-3-methyl-cyclopentyl)-acetic acid methyl ester

Diphenylphosphoryl azide (8.07 mL, 37.4 mmol), triethylamine (5.36 mL, 39 mmol), and ((1S,3R)-1-methoxycarbonylmethyl-3-methyl-cyclopentyl)-acetic acid (7.93 g, 37 mmol) were refluxed in toluene (80 mL) for 17 hours. The mixture was allowed to cool and then taken up in ethyl acetate (250 mL), washed with saturated aqueous sodium hydrogen carbonate (250 mL), brine (100 mL), and dried (MgSO₄). The solvent was removed under reduced pressure to give 7.82 g (100%) of ((1R3R)-1-isocyanatomethyl-3-methy]-cyclopentyl)-acetic acid methyl ester which was used without further purification;

IR thin film (cm⁻¹) 2264 (CN), 1732 (C═O).

### [(1R,3R)-1-(Methoxycarbonylamino-methyl)-3-methyl-cyclopentyl]-acetic acid methyl ester

((1R,3R)-1-Isocyanatomethyl-3-methyl-cyclopentyl)-acetic acid methyl ester (7.82 g, 37 mmol) was refluxed in methanol (30 mL) and toluene (80 mL) for 17 hours and then allowed to cool to room temperature. The solvent was removed under reduced pressure, and the residue was purified by chromatography (silica gel, heptane to heptane:ether 8:2) to give 2.60 g (29%) of [(1R,3R)-1-(methoxycarbonylamino-methyl)-3-methyl-cyclopentyl]-acetic acid methyl ester.

R_{f} (heptane-ethyl acetate, 1:1) 0.52:

IR thin film (cm⁻¹) 1728 (C═O), 1716 (C═O):

¹H-NMR (400 MHz. CDCl₃): δ 3.67 (6H, s, OMe, NHCO₂Me), 3.21 (1H, dd, J 7.08, 14.2. CH_{A}H_{B}NHCO2Me), 3.11 (1H, dd, J 6.10, 13.9, CH_{A}H_{B}NHCO₂Me), 2.36 (2H, s, CH₂CO₂Me), 2.05 (1H, m, CHMe). 1.86-1.46 & 1.29-1.18 (5H, m), 0.99 (3H, d, J 6.59, Me).

### ((1R,3R)-1-Aminomethyl-3-methyl-cyclopentyl)-acetic acid hydrochloride

[(1R,3R)-1-(Methoxycarbonylamino-methyl)-3-methyl-cyclopentyl]-acetic acid methyl ester (2.60 g, 37 mmol) was refluxed in a mixture of 1,4-dioxane (15 mL) and 6N Hydrochloric acid (30 mL) for 16 hours. The mixture was allowed to cool, diluted with water (80 mL), and washed with dichloromethane (2×200 mL). The aqueous layer was evaporated under reduced pressure, and the residue was recrystallized from ethyl acetate/methanol (95:5) to give 0.55 g (25%) of ((1R,3R)-1-aminomethyl-3-methyl-cyclopentyl)-acetic acid hydrochloride:

IR thin film (cm⁻¹) 1724 (C═O).

¹H-NMR (400 MHz; DMSO-d₆): δ 2.92 (1H, d, J 12.9, CH_{A}H_{B}N), 2.87 (1H, d, J 12.9, CH_{A}H_{B}N), 2.45 (1H, d, J 15.9, CH_{A}H_{B}COOH), 2.40 (1H, d, J 15.9, CH_{A}H_{B}COOH), 1.95 (1H, m), 1.84-1.72 (2H m), 1.60-1.48 (2H, m), 1.20 (1H, m), 1.04 (1H, m), 0.96 (3H, d, J6.8, Me).

### EXAMPLE 3

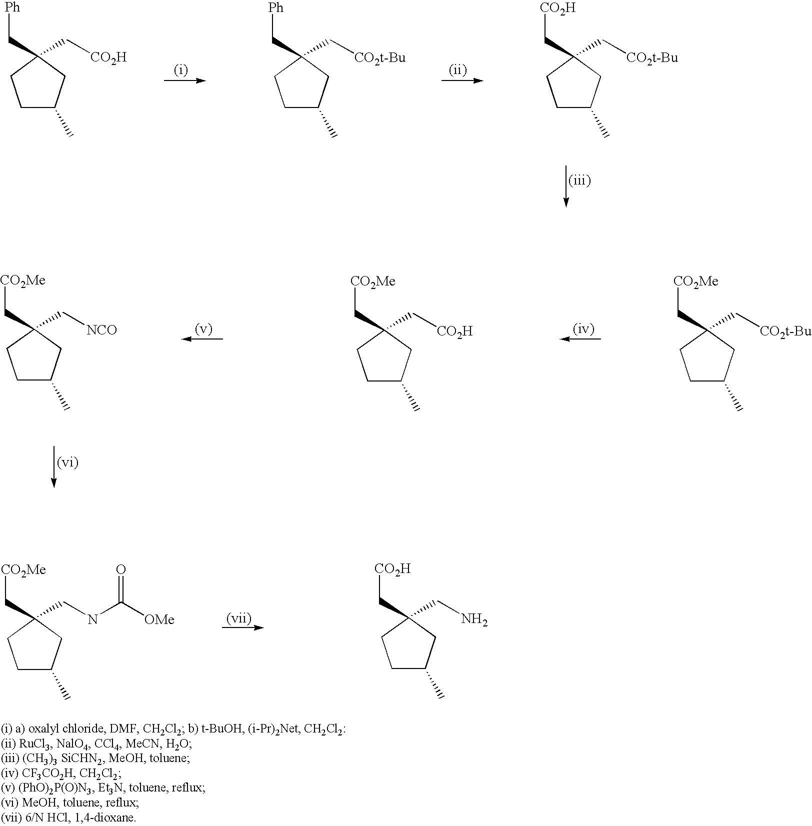

### ((1S,3R)-1-Benzyl-3-methyl-cyclopentyl)-acetic acid tert-butyl ester

Oxalyl chloride (4.14 mL, 47 mmol) was added dropwise to a stirring solution of ((1S,3R)-1-benzyl-3-methyl-cyclopentyl)-acetic acid (10 g, 43 mmol) in dichloromethane under argon at room temperature. The reaction mixture was cooled to 5° C., dimethylformamide (1 mL) was carefully added, and the mixture was allowed to warm to room temperature and stirred for a further 2 hours. The solvent was removed in vacuo and the residue diluted with dichloromethane (60 mL), 1,1-Dimethylethanol (15 mL) was carefully added to the reaction mixture under arson followed by diisopropylethylamine (11.5 mL, 65 mmol). The mixture was stirred for 17 hours and then taken up in ethyl acetate, washed with saturated aqueous sodium hydrogen carbonate (2×200 mL), and dried (MgSO₄). The solvent was removed under reduced pressure, and the residue was purified by chromatography (silica gel, eluting with a gradient of heptane to 9:1 heptane:ethyl acetate) to give 10.92 g (88%) of ((1S,3R)-1-benzyl-3-methyl-cyclopentyl)-acetic acid tert-butyl ester.

R_{f} (heptane-ethyl acetate, 9:1) 0.64:

IR thin film (cm⁻¹) 1724 (C═O).

¹H-NMR (400 MHz:CDCl₃): δ 7.29-7.17 (5H, m, Ph), 2.77 (1H, d, J 13.6, CH_{A}H_{B}Ph), 2.71 (1H, d, J 13.62 CH_{A}H_{B}Ph), 2.18 (1H s, CH_{A}H_{B}CO₂^{t}Butyl), 2.17 (1H, s, CH_{A}H_{B}CO₂^{t}Butyl), 1.49 (9H, s, CMe₃), 2.17-1.5 & 1.30-1.00 (7H, m), 1.00 (3R, d, J 6.8. CHMe).

### [(1S,3R)-1-Carboxymethyl-3-methyl-cyclopentyl]-acetic acid tert-butyl ester

((1S,3R)-1-Benzyl-3-methyl-cyclopentyl)-acetic acid tert-butyl ester (10.72 g, 37.2 mmol) and sodium periodate (124.77 g, 0.583 mol) were stirred together in carbon tetrachloride (120 mL), acetonitrile (120 mL), and water (210 mL) for 2 hours. The mixture was cooled to 0° C. and ruthenium(II) chloride (0.173 g, 0.83 mmol) was added to the reaction mixture. The reaction was allowed to warm to room temperature and stirred for 48 hours. Diethyl ether (60 mL) was added, and the mixture was then acidified to pH 2 by the addition of dilute hydrochloric acid. The mixture was extracted with ethyl acetate (2×200 mL), dried (MgSO₄), and concentrated in vacuo. The residue was purified by chromatography (silica gel, eluting with a gradient of heptane to 1:1 heptane:ethyl acetate) to give 7.01 g (73.5%) of [(1S,3R)-1-carboxymethyl-3-methyl-cyclopentyl]-acetic acid tert-butyl ester.

R_{f} (heptane-ethyl acetate, 1:1) 0.58.

IR thin film (cm⁻¹) 2953 (OH), 1726 (C═O) 1705 (C═O);

¹H-NMR (400 MHz: CDCl₃): δ 2.51 (2H, s, CH₂CO), 2.46 (2H, s, CH₂CO), 1.47 (9H, s, CMe₃), 2.05-2.15, 1.95-1.80, 1.75-1.60. 1.30-1.03 (7H, m), 1.01 (3H, d, J 6.4, Me).

### [(1S,3R)-1-Methoxycarbonylmethyl-3-methyl-cyclopentyl]-acetic acid tert-butyl ester

Trimethylsilyldiazomethane (14 mL of a 2 M solution in hexanes, 26.9 mmol) was added dropwise to a stirring solution of [(1S,3R)-1-carboxymethyl-3-methyl-cyclopentyl]-acetic acid tert-butyl ester (6.9 g. 26.9 mmol) in toluene (60 mL) and methanol (15 mL) at 10° C. under arson, and the mixture was allowed to warm to room temperature. The mixture was stirred for 2 hours, and then the solvent was evaporated under reduced pressure. The residue was taken up in ethyl acetate (200 mL). washed with saturated sodium hydrogen carbonate solution, dilute hydrochloric acid, dried (MgSO₄), and the solvent removed in vacuo. The residue was purified by chromatography (silica gel, eluting with a gradient of heptane to 95:5 heptane:ethyl acetate) to give 6.73 g (92.4%) of [(1S,3R)-1-methoxycarbonylmethyl-3-methyl-cyclopentyl]-acetic acid tert-butyl ester

R_{f} (heptane-ethyl acetate, 9:1) 0.36;

IR thin film (cm⁻¹) 1738 (C═O) 1732 (C═O);

¹H-NMR (400 MHz, CDCl₃): δ 3.65 (3H, s, OMe), 2.52 (2H, m, CH₂CO₂), 2.45 (1H, d, J 4.8, CH₂CO₂), 1.44 (9H, s, CMe₃), 2.05-1.5, 1.30-1.10 (7H, m), 1.00 (3H, d, J 6.8, Me).

### ((1R,3R)-1-Methoxycarbonylmethyl-3-methyl-cyclopentyl)-acetic acid

[(1S,3R)-1-Methoxycarbonyl methyl-3-methyl-cyclopentyl]-acetic acid tert-butyl ester (6.64 g, 24.6 mmol) and trifluoroacetic acid (10 mL) were stirred together in dichloromethane (30 mL) for 17 hours at room temperature. The mixture was carefully poured into aqueous sodium carbonate and extracted with ethyl acetate (200 mL). The aqueous was acidified to pH 1 with concentrated hydrochloric acid and re-extracted with ethyl acetate (3×200 mL), dried (MgSO₄), and concentrated in vacuo. The residue was purified by chromatography (silica gel, eluting with a gradient of heptane to 1:1 heptane:ethyl acetate) to give 5.26 g (100%) of [(1R,3R)-1-methoxycarbonylmethyl-3-methyl-cyclopentyl]-acetic acid;

R_{f} (heptane-ethyl acetate, 1:1) 0.46;

IR thin film (cm⁻¹) 2952 (OH), 1737 (C═O), 1706(C═O):

¹H-NMR (400 MHz: CDCl₃): δ 3.68 (3H, s, OMe), 2.67 (1H, d, J 15.0, CH_{A}H_{B}CO₂), δ 2.61 (1H, d, J 14.9, CH_{A}H_{B}CO₂), 2.58 (1H, d, J 14.8. CH_{A}H_{B}CO₂), 2.53 (1H, d, J 14.8. CH_{A}H_{B}CO₂), 1.93-1.81. 1.75-1.59, 1.75-1.63 (6H, m), 1.16 (1H, dd, J 19.5. 9.3), 1.01 (3H, d, J 6.35, Me).

### ((1S,3R)-1-Isocyanatomethyl-3-methyl-cyclopentyl)-acetic acid methyl ester

Diphenylphosphoryl azide (5.35 mL, 24.8 mmol), triethylamine (3.55 mL, 25.6 mmol), and [(1R,3R)-1-methoxycarbonylmethyl-3-methyl-cyclopentyl]-acetic acid (5.26 g, 24.5 mmol) were refluxed in toluene (80 mL) for 17 hours. The mixture was allowed to cool and then taken up in ethyl acetate (300 mL), washed with saturated aqueous sodium hydrogen carbonate solution (250 mL), brine (200 mL), and dried (MgSO₄). The solvent was removed under reduced pressure to give 5.19 g (100%) of ((1S,3R)-1-isocyanatomethyl-3-methyl-cyclopentyl)-acetic acid methyl ester which was used without further purification:

IR thin film (cm⁻¹) 2262 (NCO), 1732 (C═O).

### [(1S,3R)-1-(Methoxycarbonylamino-methyl)-3-methyl-cyclopentyl]-acetic acid methyl ester

((1S,3R)-1-Isocyanatomethyl-3-methyl-cyclopentyl)-acetic acid methyl ester (5.19 g. 24.5 mmol) was refluxed in methanol (30 mL) and toluene (80 mL) for 17 hours and then allowed to cool to room temperature. The solvent was removed under reduced pressure, and the residue was purified by chromatography (silica gel, heptane-ethyl acetate 9:1) to give 4.62 g (77%) of [(1S,3R)-1-(methoxycarbonylamino-methyl)-3 -methyl-cyclopentyl]-acetic acid methyl ester;

R_{f} (heptane-ethyl acetate, 1:1) 0.59.

IR thin film (cm⁻¹) 1730 (C═O).

¹H-NMR (400 MHz: CDCl₃): δ 3.68 (6H, s, OMe, NHCO₂Me), 3.27 (1H, dd, J 13.7, 6.8, CH_{A}H_{B}NHCO₂Me), 3.13 (1H, dd, J 13.9. 6.4. CH_{A}H_{B}NHCO₂Me), 2.37 (1H, d, J 13.9, CH_{A}H_{B}CO₂), 2.33 (1H, d, J 13.9. CH_{A}H_{B}CO₂), 2.09-1.99 (1H, m, CHMe), 1.88-1.76. 1.69-1.43. 1.28-1.19 (6H, m), 1.01 (3H, d, J 6.4, Me); m/z (Cl⁺) 244 (M+H, 100%).

### ((1S,3R)-1-Aminomethyl-3-methyl-cyclopentyl)-acetic acid hydrochloride

[(1S,3R)-1-(Methoxycarbonylamino-methyl)-3-methyl-cyclopentyl]-acetic acid methyl ester (2.84 g, 11.7 mmol) was refluxed in a mixture of 1,4-dioxane (15 mL) and 6N Hydrochloric acid (30 mL) for 17 hours. The mixture was allowed to cool, diluted with water (200 mL), and washed with dichloromethane (2×100 mL). The aqueous layer was evaporated under reduced pressure, and the residue was recrystallized from ethyl acetate/methanol (95:5) to give 1.28 g (53%) of ((1S,3R)-1-aminomethyl-3-methyl-cyclopentyl)-acetic acid hydrochloride,

IR thin film (cm⁻¹) 1710 (C═O):

¹H-NMR (400 MHz; DMSO-d₆): δ 2.96 (1H, d, J 12.8, CH_{A}H_{B}NH₂), 2.90 (1H, d, J 12.8, CH_{A}H_{B}NH₂) 2.40 (2H, s, CH₂COOH), 2.09-1.98 (1H, m, CHMe), 1.81-1.61. 1.51-1.43, 1.21-1.11 (5H, m), 1.04 (1H, dd, J 13.2, 10.4), 0.97 (3H, d, J 6.35, Me);

MS (ES⁺) m/z 173 (M+H, 100%), 196 (M+Na, 10%);

LCMS (Prodigy® ODS 3 50 mm×4.6 mm id column, 5% for 2 min, 5-50% over 1.5 min of acetonitrile (0.05% formic acid)/water (0.05% formic acid)) Retention

Time=0.92, 94% de; (Found: C, 49.5; H, 8.78; N, 6.3.

C₉H₁₇NO₂.1HCl.0.6H₂O requires C, 49.5; H, 8.86; N, 6.41).

1. A compound of Formula II 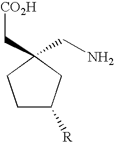
wherein R is C₁-C₁₀ alkyl or C₃-C₁₀ cycloalkyl, and pharmaceutically acceptable salts thereof, prepared by a process comprising the steps of:
a) adding a cyanoacetate of Formula 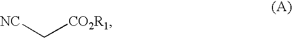
wherein R₁ is alkyl or benzyl, to a mixture of a chiral cyclopentanone of Formula 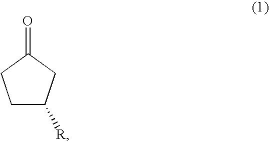
a solvent, a carboxylic acid, a Knoevenagel reaction catalyst, and stirring the mixture in the presence of a means of removing water to produce the alkene of Formula 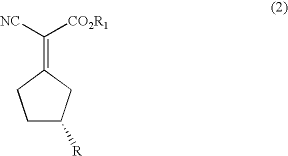
b) adding the product Step (a) above to a mixture of benzylmagnesium chloride, benzylmagnesium bromide, or benzylmagnesium iodide, in a solvent to produce the addition products of formulas 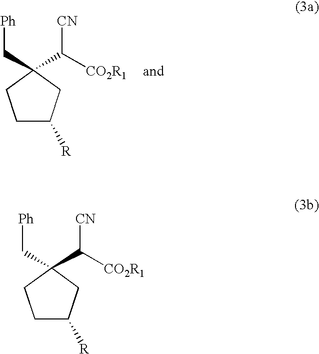
c) adding the products of Step (b) above to a mixture of a base selected from potassium hydroxide, sodium hydroxide, lithium hydroxide and cesium hydroxide, in a solvent, and stirring, and then acidifying to produce the carboxylic acids of formulas 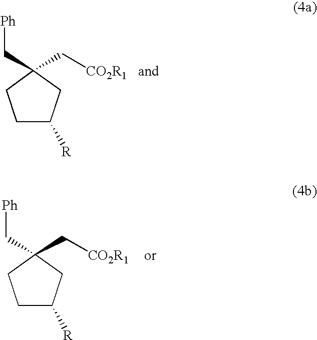
adding the products of Step (b) above to an acid mixture and stirring to produce the carboxylic acids of formulas 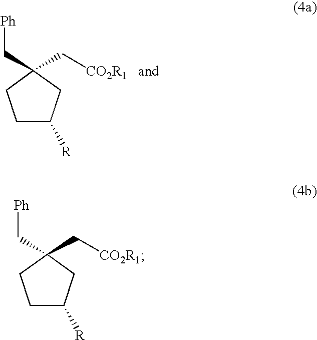
d) contacting the products of Step c) above with an amine in a solvent, and recrystallizing the salt so formed to produce the enriched diastereomer of Formula 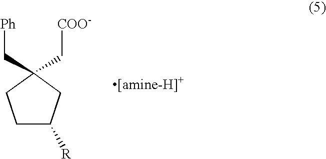
as the amine salt;
e) concerting the product of Step d) to a carboxylic acid of Formula 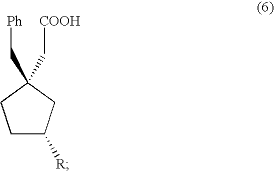
f) adding oxalyl chloride to a mixture of the product of Step e), a solvent, and N,N-dimethylformamide (DMF), and stirring to produce the acid chloride of Formula 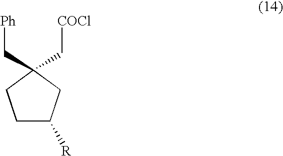
g) adding the product of Step f) to a mixture of tert-butyl alcohol, a solvent, and a tertiary amine base, and stirring to produce the ester of Formula 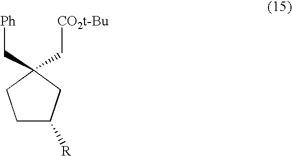
h) adding the product of Step g) to a mixture of carbon tetrachloride or ethyl acetate, and acetonitrile, water, sodium periodate, and ruthenium (III) chloride, and stirring to produce the carboxylic acid of of Formula 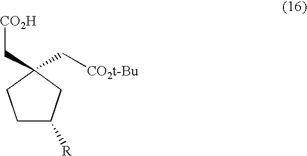
i) adding the product of Step h) to a mixture of a solvent, methanol, and (trimethylsilyl) diazomethane, and stirring to oproduce the bis ester of of Formula 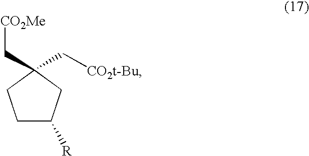
or adding the product of Step h) to a mixture of iodomethane, a solvent, and a base, and stirring to produce the bis ester of Formula 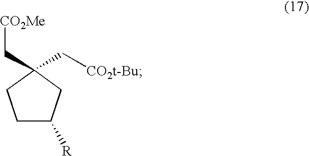
j) adding an acid to a mixture of the product from Step i) and a solvent, and stirring to produce the carboxylic acid of Formula 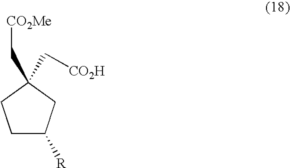
k) adding the product of Step j) to a mixture of a tertiary amine base, a solvent, and diphenylphosphoryl azide (DPPA) is added, and stirring to produce the isocyanate of Formula 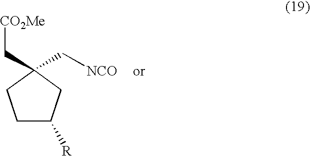
adding the product of step j) above to ethyl chloroformate or isobutyl chloroformate and a base in a solvent at a temperature of from −40° C. to 78° C., followed by adding a solution of sodium azide in water and tetrahydrofuran or acetone, followed by adding toluene or benzene, and refluxing to produce the isocyanate of Formula 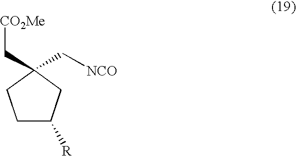
l) adding the product to Step k) to a mixture of a solvent and methanol, and stirring to produce the carbamate of Formula 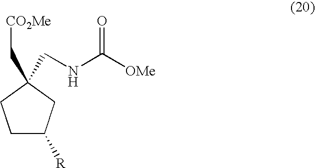
m) adding the product of Step l) to a mixture of a solvent and aqueous hydrochloric acid is added, and stirring to produce a compound of Formula 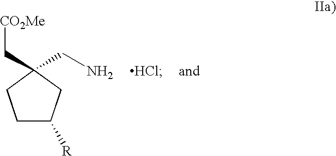
n) converting the product of step m) to a compound of Formula 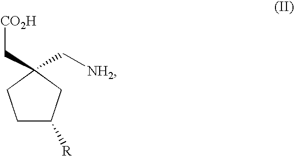
and further converting, if desired, to a pharmaceutically acceptable salt by known means.

2. A compound according to claim 1 wherein R is selected from methyl, ethyl, and n-propyl.

3. A compound according to claim 1 selected from ((1S,3R)-1-aminomethyl-3-methyl-cyclopentyl)-acetic acid and ((1S,3R)-1-aminomethyl-3-methyl-cyclopentyl)-acetic acid hydrochloride.

4. A compound selected from:
((1S,3R)-1-Aminomethyl-3-methyl-cyclopentyl)-acetic acid;
((1S,3R)-1-Aminomethyl-3-methyl-cyclopentyl)-acetic acid hydrochloride;
((1S,3R)-1-Aminomethyl-3-methyl-cyclopentyl)-acetic acid;
((1S,3R)-1-Aminomethyl-3-methyl-cyclopentyl)-acetic acid hydrochloride;
((1S,3R)-1-Aminomethyl-3-propyl-cyclopentyl)-acetic acid; and
((1S,3R)-1-Aminomethyl-3-propyl-cyclopentyl)-acetic acid hydrochloride.
